(19) United States
(12) Patent Application Publication
(10) Pub. No.: US 2010/0178684 A1
(43) Pub. Date: Jul. 15, 2010

(54) TRANSGENIC ONCOLYTIC VIRUSES AND USES THEREOF

(76) Inventors: Savio L.C. Woo, New York, NY (US); Oliver Ebert, New York, NY (US); Adolfo Garcia-Sastre, New York, NY (US); Jennifer Altomonte, New York, NY (US)

Correspondence Address: DARBY & DARBY P.C., P.O. BOX 770, Church Street Station, New York, NY, 10008-0770, US

(21) Appl. No.: 12/520,571

(22) PCT Filed: Dec. 21, 2007
(86) PCT No.: PCT/US07/88630

§ 371 Date: Jun. 22, 2009

Related U.S. Application Data
(60) Provisional application No. 60/871,448, filed on Dec. 21, 2006.

(51) Int. Cl. C12N 7/01; C12N 5/09

(52) U.S. Cl. 435/235.1; 435/375

(57) ABSTRACT

The present disclosure relates to a recombinant oncolytic virus useful for inhibiting the growth of or killing tumor cells. More specifically, the recombinant oncolytic virus contains a heterologous nucleic acid sequence encoding an inflammation suppressive gene including, but not limited to, natural killer cell inhibitor, a chemokine binding protein, and an NF-κB inhibitor. Alternatively, the recombinant oncolytic virus contains a two or more heterologous nucleic acid sequences encoding one or more inflammation suppressive genes including, but not limited to, natural killer cell inhibitor(s), one or more chemokine binding protein(s), and/or one or more NF-κB inhibitor(s). Optionally, a recombinant oncolytic virus may further comprise one or more heterologous viral internal ribosome entry site (IRES) that is neuronally-silent. Such recombinant oncolytic viruses can be used to treat singular tumors or multi-focal tumors, such as those found in hepatocellular carcinoma or other cancers.

### CROSS REFERENCE TO RELATED APPLICATIONS

This application is the U.S. National Phase of International Patent Application Serial No. PCT/U.S.07/88630, filed Dec. 21, 2007, which claims priority to U.S. Provisional Patent Application No. 60/871,448, filed Dec. 21, 2006, both of which are incorporated by reference herein in their entireties.

### GOVERNMENT SUPPORT

The work disclosed in the present application was supported, in part, by an NIH grant (CA100830). The Federal Government may have rights in certain aspects of the presently disclosed invention.

### TECHNICAL FIELD

The present disclosure relates, generally, to recombinant oncolytic viruses useful for inhibiting the growth of or killing tumor cells. Within certain embodiments, recombinant oncolytic viruses contain a heterologous nucleic acid sequence encoding a natural killer cell inhibitor or a chemokine binding protein or both and, optionally, a heterologous viral internal ribosome entry site (IRES) that is neuronally-silent. Within other embodiments, recombinant oncolytic viruses contain a heterologous nucleic acid sequence encoding an NFκB inhibitor and, optionally, a heterologous viral internal ribosome entry site (IRES) that is neuronally-silent. Such recombinant oncolytic viruses can be used to treat singular or multi-focal tumors, such as those found in hepatocellular carcinoma (HCC) and other cancers.

### BACKGROUND

Oncolytic viruses are currently being developed as a novel class of therapeutic agents for cancer treatment. Most oncolytic viruses currently used in advanced clinical trials are derived from adenovirus or Herpes Simplex Virus. Kasuya, Cancer Gene Ther. 12(9):725-36 (2005) and Rainov, Acta. Neurochir. Suppl. 88:113-23 (2003). Vectors derived from retroviruses have also been explored for their oncolytic potential due to tumor specificity owing to their selective ability to productively infect only dividing cells. Lyons et al., Cancer Gene Therapy 2(4):273-80 (1995); Logg and Kasahara, Methods Mol. Biol. 246:499-525 (2004); and Finger et al., Cancer Gene Therapy 12(5):464-74 (2005). More recently, RNA viruses (including, for example, Reoviruses, Newcastle Disease Viruses, Measles Viruses, and Vesicular Stomatitis Viruses) exhibiting inherent tumor specificity have been exploited as oncolytic agents for the treatment of cancer. Kirn et al., Nat Med 7(7):781-787 (2001).

Vesicular stomatitis virus (VSV) is an enveloped, single-strand RNA virus belonging to the family Rhabdoviridae, genus Vesiculovirus, with 16 distinct serotypes, of which six can cause animal or human disease. Rose and Whitt, “Fields Virology” 1221-1242 (D. M. Knipe and P. M. Howley, Philadelphia, Lippincott Williams & Wilkins (2001). VSV causes a vesicular disease in domestic animals resembling foot-and-mouth disease, with excess salivation, fever and blisters/vesicles in the oronasal region and hooves. A high percentage of people living in endemic areas such as central and southwestern United States and Canada may also be infected. Rodriguez, Virus Res. 85:211-19 (2002).

Transmission of VSV is believed to be mediated by an insect vector such as the phlebotomine sand-fly. Shelokov and Peralta, Am. J. Epidemiol. 86:149-57 (1967). The viral illness in humans is generally sub-clinical resulting in the induction of interferons and neutralizing antibodies, which are effective against the virus. Occasionally, VSV can cause a mild illness in humans with oral vesicular lesions, fever, malaise, and pharyngitis. Fields and Hawkins, New Engl. J. Med. 277:989-94 (1967). Two cases of VSV meningoencephalitis have been reported in children. Quinol et al., Am. J. Trop. Med. Hyg. 39:312-314 (1988).

The envelope G-protein of VSV binds to the surface of most insect and mammalian cell types accounting for the wide tissue tropism for VSV. Virol replication is inhibited in normal cells due to the induction of cellular interferons, thereby sparing the cell from cytopathic destruction. In tumor cells, however, viral replication is uninhibited because of defects in the cellular interferon pathways. Such uninhibited viral replication typically results in apoptotic tumor cell death. Stojdl et al., Natl. Med. 6:821-825 (2000). The oncolytic property of VSV, therefore, makes this virus a potentially effective agent for selective anti-tumor treatment. Giedlin et al., Cancer Cell 4:21-43 (2003). Thus, VSV and recombinant VSV vectors are currently being developed as potent oncolytic agents for the treatment of cancers. Stojdl et al., Nat Med 6(7):821-825 (2000). VSV Vectors have, for example, been used to treat an orthotopic model of multi-focal hepatocellular carcinoma (HCC) in the livers of syngeneic and immune-competent rats through hepatic artery infusion, which has led to tumor-selective virus replication, oncolysis, tumor-regression, and modest survival prolongation. Ebert et al., Cancer Research 63(13):611-613 (2003). VSV, and other oncolytic viruses, have also been used for the treatment of colorectal cancers (Shinozaki et al., Int. J. Cancer 114(4):659-64 (2005)); breast cancers (Ebert et al., Cancer Gene Ther. 12(4):350-8 (2005)); lung cancers (Li et al., Int. J. Cancer 112(1):143-9 (2004)); head and neck cancers (Shin et al., Otolaryngol. Head Neck Surg. 136(5):811-7 (2007)); brain cancers (Zhang et al., Exp. Oncol. 29(2):85-93 (2007)); and leukemias (Cesaire et al., Oncogene 25(3):349-58 (2006)).

Recombinant VSV (rVSV) can be generated using a “reverse genetics” system for negatively stranded RNA viruses. rVSV encoding marker genes, such as those encoding betagalactosidase or green fluorescent protein (rVSV-G), have been produced and have been tested in a rat model of established syngeneic multifocal HCC. Shinozaki et al., Mol. Ther. 9(3):368-76 (2004).

The tumoricidal effects of oncolytic VSV have been amplified through syncytia induction by incorporating into VSV a fusogenic membrane glycoprotein gene (F) from the heterologous Newcastle Disease Virus (rVSV-F). Ebert et al., Cancer Research 63(13):611-613 (2003) and Ebert et al., Cancer Research 64:3265-3270 (2004). Although statistically significant survival advantage has been achieved in animals bearing multi-focal HCC in the liver, long-term survival has not been achieved in most treated rats as intratumoral virus replication appears to be rapidly suppressed by an anti-viral inflammatory response in the immune-competent host. Additionally, limb paralysis secondary to VSV replication in neurons has been observed in some of the animals treated with the vector at doses above the maximum tolerated dose (MTD). Most wild-type strains of VSV are known to be relatively poor inducers of IFN (Marcus et al., J. Virol. 72:542-549 (1998)).

The VSV matrix (M) protein is a virulence factor that is capable of inhibiting host gene expression at the level of transcription (Ferran and Lucas-Lenard, J. Virol. 71:371-377 (1997) and Ahmed et al., J. Virol. 77:4646-4657 (2003)) as well as the nuclear-cytoplasmic transport of host RNAs and protein (Petersen et al., Mol. Cell. Biol. 20:8590-8601 (2000) and von Kobbe et al., Mol. Cell. 6:1243-1252 (2000)). Recently, Stojdl et al., Cancer Cell 4(4):263-275 (2003) reported that VSV mutants containing either one (M51R) or two (V221F and S226R) amino acid substitutions in the viral matrix (M) protein are potent inducers of IFN and are safe in mice after repeated systemic administrations at high doses.

The potential of a recombinant VSV containing a deletion at position 51 within the M protein (VSV(MΔ51)) as an oncolytic agent for the treatment of breast cancer metastases has recently been investigated via intravenous administration in an immune-competent mouse model system. Ebert et al., Cancer Gene Therapy 12(4):350-8 (2005). The results confirmed that the M-mutant is a much safer oncolytic virus than is wild-type VSV. Unfortunately, however, the intratumoral replication of VSV(MΔ51) is attenuated in comparison to wild-type VSV, which results in a significantly reduced oncolytic potency of VSV(MΔ51).

Because of their vastly improved safety profiles, however, VSV(MΔ51) based vectors are particularly attractive candidates for clinical translational applications. The matrix (M) protein of VSV is not only a structural protein necessary for virus assembly, but also a virulence factor of VSV. The VSVM protein interferes with host cell gene expression in infected cells by blocking mRNA export to the cytosol. Gaddy and Lyles, J. Virol. 79:4170-4179 (2005). It has been reported that deletion of its 51st amino acid results in the loss of its ability to block cellular mRNA transport, leading to elevated interferon and cytokine expression in the virus infected cells. An enhanced IFN response attenuates virus replication in normal cells, thus reducing VSV-related toxicity. Tumor cells with their attenuated IFN responsiveness, however, remain susceptible to VSV(MΔ51) replication and cytolytic killing.

The general applicability of VSV(MΔ51) as an effective agent to kill multiple tumor types in vitro has been demonstrated by Bell's group, and it is highly lytic in most of the NCI panel of 60 human cancer cell lines. Stojdl et al., Cancer Cell 4(4):263-275 (2003). Their studies further demonstrated that infection with VSV(MΔ51) could establish an antiviral state in the recipient animals that protects against toxicities normally associated with infection by wild type VSV. This observation has been confirmed in an immune-competent mouse model of metastatic breast cancer, where the MTD of the rVSV(M51R)-LacZ was elevated by at least 100-fold over that of an equivalent virus, rVSV-LacZ. Ebert et al., Cancer Gene Therapy 12(4):350-8 (2005).

In immune-competent hosts, the duration of intratumoral replication of VSV(MΔ51), and other oncolytic viruses, is limited by a rapid anti-viral inflammatory response that precedes a neutralizing anti-viral antibody response. Cellular inflammatory processes are mediated by chemo-attractants called chemokines (Schall and Bacon, Curr. Opin. Immunol. 6:865-873 (1994)), which is a large family of small signaling peptides that bind to G-protein-coupled receptors on target immune cells. Chemokines induce the chemtaxis of immune cells to the sites of inflammation and play a central role in the host defense against invading viruses, including the oncolytic viruses. Rollins, Blood 90:909-928 (1997) and Baggiolini, Nature 392:565-568 (1998). During the early phase of virus infection, innate cells (neutrophils and natural killer cells) are the first to infiltrate the infected site after VSV infection. The first phase of chemokine expression corresponds to positive staining for neutrophils (peak, 36 h post-infection) (Bi et al., J Virol. 69(10):6466-72 (1995) and infiltrating NK cells (peak, approximately 3-4 days post-infection). Chen et al., J. Neuroimmunol. 120(1-2):94-102 (2001) and Ireland et al., Virol Immunol. 19:536-545 (2006). The second phase of expression corresponds to the infiltration of macrophages (Christian et al., Virol Immunol. 9:195-205 (1996) and CD4+ and CD8+ T cells, which peak after one week (Huneycutt et al., J. Virol. 67:6698-6706 (1993)). Since intratumoral VSV replication is inhibited after 1-3 days of virus infusion, neutrophil and NK cell recruitment is important in inhibiting virus propagation during early infection. Chen et al., Neuroimmunol. 120(1-2):94-102 (2001) and Ireland et al., Virol Immunol. 19:536-545 (2006). Thus, the utility of many oncolytic viruses as anti-tumor agents, as exemplified by the recombinant VSV(MΔ51) virus, is limited by the host's chemokine-mediated inflammatory responses.

The inflammatory response to virus challenge is characterized by the migration and activation of leukocytes, which initiate the earliest phases of antiviral immune activation. Zinkernagel, Science 271:173-178 (1996). The larger DNA viruses encode immunomodulatory proteins, which interact with a wide spectrum of immune effector molecules, as a method of evading this response. McFadden and Graham, Semin Virol. 5:421-429 (1994) and Alcami, Nature Immunology 3:36-50 (2003). One such mechanism involves the production of secreted chemokine binding proteins that bear no sequence homology to host proteins, yet function to competitively bind and/or inhibit the interactions of chemokines with their cognate receptors (Seet and McFadden, J. Leukocyte Biol. 72:24-34 (2002)) thereby suppressing the chemotaxis of inflammatory cells to the infected sites. The large DNA viruses, such as the poxviruses and herpesviruses, have evolved such mechanisms to undermine the normal functioning of the chemokine network in the host.

In particular, certain orthopoxviruses, such as vaccinia virus and myxoma virus, express members of the T1/35 kDa family of secreted proteins which bind with members of the CC and CXC superfamilies of chemokines, and effectively block leukocyte migration in vivo. Graham, et al., Virology 229:12-24 (1997). More recently, it was demonstrated that ectromelia virus (EV) expresses a soluble, secreted 35 kDa viral chemokine binding protein (EV35) with properties similar to those of homologous proteins from the T1/35 kDa family. It was demonstrated in vitro that EV35 specifically and effectively sequesters and binds CC chemokines, and it is speculated that in vivo chemokine binding activity would inhibit migration of monocytes, basophils, eosinophils, and lymphocytes. Smith et al., Virology 236:316-327 (1997); Baggiolini, “The Chemokines,” 1-11 (ed. I. Lindley; Plenum, NY; 1993); and Baggiolini, Nature 392:565-568 (1998).

There remains an unmet need in the art for oncolytic viruses that are capable of evading the host's chemokine-mediated inflammatory responses and, as a consequence, exhibit improved anti-tumor activity.

### SUMMARY

The present disclosure fulfills these and other related needs by providing recombinant oncolytic viruses, which exhibit improved anti-tumor activity, owing to the capability of the recombinant oncolytic viruses to evade the host's chemokine-mediated inflammatory responses. Thus, within certain embodiments, the present disclosure provides recombinant oncolytic viruses having one or more nucleic acid sequences that encode immunomodulatory polypeptides, such as polypeptides that attenuate the innate immune response or inflammatory response.

In one aspect, the instant disclosure provides recombinant oncolytic viruses having a heterologous nucleic acid sequence, encoding an inhibitor of inflammatory or innate immune cell migration or function, such as a natural killer cell inhibitor, a chemokine binding protein, or an NF-κB inhibitory protein. Within certain embodiments, the heterologous nucleic acid sequence encodes one or more natural killer cell inhibitor. Within other embodiments, the heterologous nucleic acid sequence encodes one or more chemokine binding protein. Within yet other embodiments, the heterologous nucleic acid sequence encodes one or more NF-κB inhibitory protein.

Within other aspects, the recombinant oncolytic viruses comprise two or more heterologous nucleic acid sequences encoding one or more natural killer cell inhibitor(s), one or more chemokine binding protein(s), and/or one or more NF-κB inhibitory protein(s). For example, within some embodiments, the recombinant oncolytic virus has a heterologous nucleic acid sequence that encodes a natural killer cell inhibitor and a heterologous nucleic acid sequence that encodes a chemokine binding protein. Within other embodiments, the recombinant oncolytic virus has a heterologous nucleic acid sequence that encodes a natural killer cell inhibitor and a heterologous nucleic acid sequence that encodes an NF-κB inhibitory protein. Within yet other embodiments, the recombinant oncolytic virus has a heterologous nucleic acid sequence that encodes a chemokine binding protein and a heterologous nucleic acid sequence that encodes an NF-κB inhibitory protein. The natural killer cell inhibitor, chemokine binding protein, and/or NF-κB inhibitory protein may be a viral, bacterial, fungal, parasitic, or eukaryotic polypeptide.

The oncolytic virus may be selected from the group consisting of vesicular stomatitis virus (VSV), Newcastle disease virus (NDV), retrovirus, reovirus, measles virus, Sinbis virus, influenza virus, herpes simplex virus, vaccinia virus, and adenovirus, or the like, or a recombinant variant thereof. In one embodiment, the oncolytic virus is VSV or a recombinant variant thereof as exemplified herein by VSV(MΔ51). In another embodiment, the modified oncolytic virus is NDV or a recombinant variant thereof.

The heterologous nucleic acid sequence that encodes a chemokine binding protein may, for example, be an equine herpesvirus-1 glycoprotein G (gG_{EHV-1} protein), a murine gamma herpesvirus-68 M3 (mGHV-M3), an orthopoxvirus T1/35 kDa protein, an ectromelia virus (EV) 35 kDa protein (EV35), a Schistosoma mansoni CKBP (smCKBP), a poxvirus CKBP, a myxoma M-T7 CKBP, a human erythroleukemic (HEL) cell CKBP. In a related embodiment, the encoded chemokine binding protein is truncated, lacks a transmembrane domain, is secreted, or any combination thereof. For example, an oncolytic virus of the present disclosure may be a recombinant VSV(MΔ51) virus comprising one or more of equine herpesvirus-1 glycoprotein G, murine gamma herpesvirus-68 M3, orthopoxvirus T1/35 kDa protein, and/or ectromelia vines (EV) 35 kDa protein (EV35). Exemplified by the present disclosure is a recombinant VSV(MΔ51) virus comprising a murine gamma herpesvirus-69 M3, which is designated VSV(MΔ51)-M3.

In another embodiment, the heterologous nucleic acid sequence that encodes a natural killer cell inhibitor may, for example, be a UL141 polypeptide of human cytomegalovirus (CMV), an M155 polypeptide of murine CMV, or a K5 polypeptide of Kaposi's sarcoma-associated herpes virus. In a related embodiment, the encoded natural killer cell inhibitor is truncated or lacks a transmembrane domain or is secreted or any combination thereof.

In yet another embodiment, the heterologous nucleic acid sequence that encodes an NF-κB inhibitory protein may, for example, be an A238L protein encoded by African Swine Fever Virus (ASFV). Alternatively, the heterologous nucleic acid sequence that encodes an NF-κB inhibitory protein may be an A52R protein or an N1L protein encoded by a poxvirus; a Vpu accessory protein encoded by human immunodeficiency virus (HIV); or an ORF2 protein encoded by Torque teno virus. In a related embodiment, the encoded NF-κB inhibitory protein is truncated or lacks a transmembrane domain or is secreted or any combination thereof.

In still another embodiment, the recombinant oncolytic virus further comprises one or more heterologous viral internal ribosome entry site (IRES) that is neuronally-silent and operably linked to at least one nucleic acid sequence that encodes an oncolytic virus polypeptide needed for virus gene expression, replication or propagation, such as a polymerase (e.g., viral RNA-dependent RNA polymerase or DNA polymerase); a structural protein (e.g., nucleocapsid protein, phosphoprotein, or matrix protein); or a glycoprotein (e.g., envelope protein). In a further embodiment, the recombinant oncolytic virus has two or three IRESs and each is operably linked to a different nucleic acid sequence that encodes an oncolytic virus polypeptide. For exmple, one IRES may be linked to an oncolytic virus polymerase and a second IRES may be linked to a structural protein or a glycoprotein. In yet a further embodiment, the recombinant oncolytic virus has a first IRES operably linked to a nucleic acid sequence that encodes an oncolytic virus polymerase; a second IRES operably linked to a nucleic acid sequence that encodes an oncolytic virus glycoprotein; and a third IRES operably linked to a nucleic acid sequence that encodes an oncolytic virus structural protein. In another embodiment, the IRES is a picornavirus IRES, such as a type I IRES from a Rhinovirus, such as a human Rhinovirus 2, or a Foot and Mouth Disease virus or any combination thereof.

In any of the embodiments disclosed herein, the recombinant oncolytic virus may further have a nucleic acid sequence encoding an NDV fusogenic protein, preferably an NDV fusogenic protein that has an L289A mutation. In a related embodiment, the recombinant oncolytic virus is capable of inducing syncytia formation.

In another aspect, the instant disclosure provides a method of inhibiting the growth or promoting the killing of a tumor cell, comprising administering a recombinant oncolytic virus according to this disclosure at a multiplicity of infection sufficient to inhibit the growth or kill the tumor cell. In certain embodiments, the tumor cell is a hepatocellular carcinoma (HCC) cell, and the HCC cell can be in vivo, ex vivo, or in vitro. In another embodiment, the recombinant oncolytic virus is administered intravascularly into a vein or an artery. For example, in the case of a hepatic tumor, the oncolytic virus is administered to a hepatic artery via an in-dwelling medical device such as a catheter. In a further embodiment, the recombinant oncolytic virus is administered intravascularly, intratumorally, or intraperitoneally. In still further embodiments, an interferon, such as interferon-α or pegylated interferon, is administered prior to administering the recombinant oncolytic virus.

In yet another aspect, the present disclosure provides methods for the treatment of a cancer in a human patient. Such methods comprise the step of administering one or more oncolytic virus as described herein at an MOI that is sufficient to retard the growth of and/or kill a tumor cell in the human patient. Such methods are exemplified herein by methods for the treatment of a cancer in a human patient, which method comprises the step of administering a recombinant VSV virus, such as the recombinant VSV(MΔ51)-M3 virus and the recombinant VSV-gG virus.

It will be understood that recombinant oncolytic viruses described herein will find utility in the treatment of a wide range of tumor cells or cancers including, for example, breast cancer (e.g., breast cell carcinoma), ovarian cancer (e.g., ovarian cell carcinoma), renal cell carcinoma (RCC), melanoma (e.g., metastatic malignant melanoma), prostate cancer, colon cancer, lung cancer (including small cell lung cancer and non-small cell lung cancer), bone cancer, osteosarcoma, rhabdomyosarcoma, leiomyosarcoma, chondrosarcoma, pancreatic cancer, skin cancer, fibrosarcoma, chronic or acute leukemias including acute lymphocytic leukemia (ALL), adult T-cell leukemia (T-ALL), acute myeloid leukemia, chronic myeloid leukemia, acute lymphoblastic leukemia, chronic lymphocytic leukemia, lymphangiosarcoma, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), Castleman's disease, Kaposi's Sarcoma, hemangiosarcoma, multiple myeloma, Waldenstrom's macroglobulinemia and other B-cell lymphomas, nasopharangeal carcinomas, head or neck cancer, myxosarcoma, liposarcoma, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, cervical carcinoma, vaginal carcinoma, vulvar carcinoma, transitional cell carcinoma, esophageal cancer, malignant gastrinoma, small intestine cancer, cholangiocellular carcinoma, adenocarcinoma, endocrine system cancer, thyroid gland cancer, parathyroid gland cancer, adrenal gland cancer, sarcoma of soft tissue, urethral, penile cancer, testicular cancer, malignant teratoma, solid tumors of childhood, bladder cancer, kidney or ureter cancer, carcinoma of the renal pelvis, malignant meningioma, neoplasm of the central nervous system (CNS), tumor angiogenesis, spinal axis tumor, pituitary adenoma, epidermoid cancer, squamous cell cancer, environmentally induced cancers including those induced by asbestos, e.g., mesothelioma, and combinations of these cancers. The present disclosure is further exemplified by the treatment of hepatocellular carcinoma (HCC) with the recombinant oncolytic virus VSV(MΔ51)-M3. It will be understood, however, that a wide variety of recombinant oncolytic viruses comprising one or more natural killer cell inhibitor(s), one or more chemokine binding protein(s), and/or one or more NF-κB inhibitory protein(s) as described herein may be suitably employed for the treatment of many distinct tumors, cancers, and other proliferative diseases.

Thus, in one embodiment the invention provides a recombinant oncolytic virus, comprising an oncolytic virus or a recombinant variant of an oncolytic virus and a heterologous nucleic acid sequence encoding an inhibitor of inflammatory or innate immune cell migration or function, wherein said heterologous nucleic acid sequence is incorporated within the genetic material of said oncolytic virus or recombinant variant of an oncolytic virus.

In one embodiment, said oncolytic virus is selected from the group consisting of vesicular stomatitis virus (VSV), Newcastle disease virus (NDV), retrovirus, reovirus, measles virus, Sinbis virus, influenza virus, herpes simplex virus, vaccinia virus, and adenovirus. In one embodiment, said oncolytic virus is vesicular stomatitis virus (VSV). In one embodiment, said recombinant variant of an oncolytic virus is a recombinant variant of a virus selected from the group consisting of vesicular stomatitis virus (VSV), Newcastle disease virus (NDV), retrovirus, reovirus, measles virus, Sinbis virus, influenza virus, herpes simplex virus, vaccinia virus, and adenovirus. In one embodiment, said recombinant variant of an oncolytic virus is VSV(MΔ51).

In one embodiment, said recombinant oncolytic virus is VSV(MΔ51)-gG. In another embodiment, said recombinant oncolytic virus is VSV(MΔ51)-M3.

In one embodiment, said inhibitor of inflammatory cell migration or function is selected from the group consisting of a natural killer cell inhibitor, a chemokine binding protein, and an NF-κB inhibitor. In one embodiment, said natural killer cell inhibitor, said chemokine binding protein, or said NF-κB inhibitor is a viral protein, a bacterial protein, a fungal protein, a parasitic protein, or a eukaryotic protein. In one embodiment, said inhibitor of inflammatory cell migration or function is a chemokine binding protein or a truncated variant thereof. In one embodiment, said inhibitor of inflammatory cell migration or function is a natural killer cell inhibitor or a truncated variant thereof. In one embodiment, said chemokine binding protein is selected from the group consisting of an equine herpes virus-1 glycoprotein G (gG_{EHV-1} protein), a murine gamma herpesvirus-68 M3 (mGHV-M3), a Schistosoma mansoni CKBP (smCKBP), a poxvirus CKBP, a myxoma M-T7 CKBP, a human erythroleukemic (HEL) cell CKBP, an orthopoxvirus T1/35 kDa protein, and an ectromelia virus (EV) 35 kDa protein (EV35). In one embodiment, said chemokine binding protein is a murine gamma herpesvirus-68 M3 (mGHV-M3). In one embodiment, said chemokine binding protein is an equine herpes virus-1 glycoprotein G (gG_{EHV-1} protein). In one embodiment, said natural killer cell inhibitor is selected from the group consisting of a UL141 polypeptide of human cytomegalovirus (CMV), an M155 polypeptide of murine CMV, and a K5 polypeptide of Kaposi's sarcoma-associated herpes virus. In one embodiment, said inhibitor of inflammatory cell migration or function is an NF-κB inhibitor or a truncated variant thereof. In one embodiment, said NF-κB inhibitor is selected from the group consisting of an A238L protein encoded by African Swine Fever Virus (ASFV), an A52R protein encoded by a poxvirus, an N1L protein encoded by a poxvirus, a Vpu accessory protein encoded by a human immunodeficiency virus (HIV), and an ORF2 protein encoded by Torque teno virus.

In one embodiment, a recombinant oncolytic virus of the invention further comprises a heterologous viral internal ribosome entry site (IRES) that is neuronally-silent and operably linked to at least one nucleic acid sequence that encodes an oncolytic virus polypeptide. In one embodiment, the oncolytic virus polypeptide is one or more of an oncolytic virus polymerase, an oncolytic virus structural protein, or an oncolytic virus glycoprotein. In one embodiment, the recombinant oncolytic virus comprises two or more IRESs and each is operably linked to a different nucleic acid sequence that encodes an oncolytic virus polypeptide. In one embodiment, the recombinant oncolytic virus has a first IRES operably linked to a nucleic acid sequence that encodes an oncolytic virus polymerase, and a second IRES operably linked to a nucleic acid sequence that encodes an oncolytic virus structural protein or glycoprotein. In one embodiment, the recombinant oncolytic virus has a first IRES operably linked to a nucleic acid sequence that encodes an oncolytic virus polymerase; a second IRES operably linked to a nucleic acid sequence that encodes an oncolytic virus glycoprotein; and a third IRES operably linked to a nucleic acid sequence that encodes an oncolytic virus structural protein. In one embodiment, the IRES is a picornavirus IRES. In one embodiment, the picornavirus IRES is a Rhinovirus IRES or a Foot and Mouth Disease virus IRES.

In yet another embodiment, the invention provides a recombinant oncolytic virus, comprising an oncolytic virus or a recombinant variant of an oncolytic virus and a heterologous nucleic acid sequence encoding an inhibitor of inflammatory or innate immune cell migration or function, wherein said heterologous nucleic acid sequence is incorporated within the genetic material of said oncolytic virus or recombinant variant of an oncolytic virus, said recombinant oncolytic virus further comprising a heterologous nucleic acid sequence encoding a viral internal ribosome entry site (IRES) that is neuronally-silent and operably linked to a nucleic acid sequence that encodes an oncolytic virus polypeptide. In one embodiment, said oncolytic virus is selected from the group consisting of vesicular stomatitis virus (VSV), Newcastle disease virus (NDV), retrovirus, reovirus, measles virus, Sinbis virus, influenza virus, herpes simplex virus, vaccinia virus, and adenovirus. In one embodiment, said recombinant variant of an oncolytic virus is a recombinant variant of a virus selected from the group consisting of vesicular stomatitis virus (VSV), Newcastle disease virus (NDV), retrovirus, reovirus, measles virus, Sinbis virus, influenza virus, herpes simplex virus, vaccinia virus, and adenovirus. In one embodiment, said inhibitor of inflammatory cell migration or function is selected from the group consisting of a natural killer cell inhibitor, a chemokine binding protein, and an NF-κB inhibitor. In one embodiment, said natural killer cell inhibitor, said chemokine binding protein, or said NF-κB inhibitor is a viral protein, a bacterial protein, a fungal protein, a parasitic protein, or a eukaryotic protein. In one embodiment, said inhibitor of inflammatory cell migration or function is a chemokine binding protein or a truncated variant thereof. In one embodiment, said chemokine binding protein is selected from the group consisting of an equine herpes virus-1 glycoprotein G (gG_{EHV-1} protein), a murine gamma herpesvirus-68 M3 (mGHV-M3), a Schistosoma mansoni CKBP (smCKBP), a poxvirus CKBP, a myxoma M-T7 CKBP, a human erythroleukemic (HEL) cell CKBP, an orthopoxvirus T1/35 kDa protein, and an ectromelia virus (EV) 35 kDa protein (EV35). In one embodiment, said chemokine binding protein is an equine herpes virus-1 glycoprotein G (gG_{EHV-1} protein) or a murine gamma herpesvirus-68 M3 (mGHV-M3). In one embodiment, said inhibitor of inflammatory cell migration or function is a natural killer cell inhibitor or a truncated variant thereof. In one embodiment, said natural killer cell inhibitor is selected from the group consisting of a UL141 polypeptide of human cytomegalovirus (CMV), an M155 polypeptide of murine CMV, and a K5 polypeptide of Kaposi's sarcoma-associated herpes virus. In one embodiment, said inhibitor of inflammatory cell migration or function is an NF-κB inhibitor or a truncated variant thereof. In one embodiment, said NF-κB inhibitor is selected from the group consisting of an A238L protein encoded by African Swine Fever Virus (ASFV), an A52R protein encoded by a poxvirus, an N1L protein encoded by a poxvirus, a Vpu accessory protein encoded by a human immunodeficiency virus (HIV), and an ORF2 protein encoded by Torque teno virus. In one embodiment, said oncolytic virus is vesicular stomatitis virus (VSV). In one embodiment, said recombinant variant of an oncolytic virus is VSV(MΔ51). In one embodiment, the oncolytic virus polypeptide is one or more of an oncolytic virus polymerase, an oncolytic virus structural protein, or an oncolytic virus glycoprotein. In one embodiment, the recombinant oncolytic virus comprises two or more IRESs and each is operably linked to a different nucleic acid sequence that encodes an oncolytic virus polypeptide. In one embodiment, the recombinant oncolytic virus has a first IRES operably linked to a nucleic acid sequence that encodes an oncolytic virus polymerase, and a second IRES operably linked to a nucleic acid sequence that encodes an oncolytic virus structural protein or glycoprotein. In one embodiment, the recombinant oncolytic virus has a first IRES operably linked to a nucleic acid sequence that encodes an oncolytic virus polymerase; a second IRES operably linked to a nucleic acid sequence that encodes an oncolytic virus glycoprotein; and a third IRES operably linked to a nucleic acid sequence that encodes an oncolytic virus structural protein. In one embodiment, the IRES is a picornavirus IRES. In one embodiment, the picornavirus IRES is a Rhinovirus IRES or a Foot and Mouth Disease virus IRES.

In another embodiment, a recombinant oncolytic virus of the invention further comprises a nucleic acid sequence encoding an NDV fusogenic protein. In one embodiment, the NDV fusogenic protein has an L289A mutation. In one embodiment, the oncolytic virus is capable of inducing syncytia formation.

In another embodiment, the invention provides a method of inhibiting the growth or promoting the killing of a tumor cell, said method comprising the step of contacting said tumor cell with a recombinant oncolytic virus of the invention at a multiplicity of infection sufficient to inhibit the growth or kill the tumor cell. In one embodiment, said tumor cell is selected from the group consisting of a hepatocellular carcinoma (HCC) cell, a colorectal cancer cell, a breast cancer cell, a lung cancer cell, a head and neck cancer cell, a brain cancer cell, a leukemia cell, a prostate cancer cell, a bladder cancer cell, and an ovarian cancer cell. In one embodiment, said tumor cell is a hepatocellular carcinoma (HCC) cell. In one embodiment, said tumor cell is in vivo, ex vivo, or in vitro. In one embodiment, the recombinant oncolytic virus is administered intraperitoneally. In one embodiment, the recombinant oncolytic virus is administered parenterally. In one embodiment, the parenteral administration is into a vein. In one embodiment, the parenteral administration is into an artery. In one embodiment, the vascular administration is via an in-dwelling medical device. In one embodiment, the recombinant oncolytic virus is administered intratumorally. In one embodiment, the method further comprises the step of contacting said tumor cell with interferon.

1. In yet another embodiment, the invention provides a method for the treatment of a cancer in a human patient, said method comprising the step of administering to said human patient a recombinant oncolytic virus of the invention at a multiplicity of infection sufficient to inhibit the growth or kill the tumor cell. In one embodiment, said tumor cell is selected from the group consisting of a hepatocellular carcinoma (HCC) cell, a colorectal cancer cell, a breast cancer cell, a lung cancer cell, a head and neck cancer cell, a brain cancer cell, a leukemia cell, a prostate cancer cell, a bladder cancer cell, and an ovarian cancer cell. In one embodiment, said tumor cell is a hepatocellular carcinoma (HCC) cell. In one embodiment, the recombinant oncolytic virus is administered intraperitoneally. In one embodiment, the recombinant oncolytic virus is administered vascularly. In one embodiment, the vascular administration is into a vein. In one embodiment, the parenteral administration is into an artery. In one embodiment, the vascular administration is via an in-dwelling medical device. In one embodiment, the recombinant oncolytic virus is administered intratumorally. In one embodiment, the method further comprises the step of administering interferon to said human patient. In one embodiment, said recombinant oncolytic virus and said interferon are administered concurrently or sequentially.

These and other embodiments, features, and advantages of the disclosure will become apparent from the detailed description and the appended claims set forth herein below. All literature and patent references cited throughout the application are hereby incorporated by reference in their entireties.

### BRIEF DESCRIPTION OF THE DRAWINGS

FIG. 1A shows a wild-type vesicular stomatitis virus (VSV) genome map depicting the five viral genes: nucleocapsid (N), phosphoprotein (P), matrix protein (M), glycoprotein (G), and polymerase (L). The arrows point to the 3′-untranslated regions that can be used to insert transgenes—* the 3′-untranslated region of G is known to be a stable site for transgene insertion. Ebert et al., Cancer Res. 64:3265 (2004). The bars above the genome depicts the relative transcriptional levels of each VSV gene when expressed in infected cells (i.e., the more bars, the greater the expression).

FIG. 1B shows a schematic representation of a recombinant VSV (rVSV-gG) construct expressing a viral chemokine binding protein gene, equine herpesvirus 1 glycoprotein G (gG_{EHV1}). Shown is a full-length pVSV plasmid containing the five VSV genes, and a bicistronic construct containing the gG_{EHV1} and firefly luciferase (Luc) with a promiscuous intervening internal ribosome entry site (IRES) from encephalomyocarditis virus (EMCV). The transgenes are preceded by a VSV transcription termination signal, an intergenic region, and a transcription start signal (SEQ ID NO: 13), which are inserted into the 3′-untranslated region of the VSVG gene.

FIG. 1C shows rVSV constructs containing viral anti-inflammatory genes and IRES elements to direct the translation of VSVG and VSVL mRNAs. The VSV full-length plasmid is shown. At the 5′-untranslated regions of the VSVG or VSVL transcripts, a heterologous transgene (vTG) is inserted, followed by an IRES (e.g., neuronally-silent), to generate two different rVSV vectors or one that contains both vTGs and both IRESs. Transcription start signal disclosed as SEQ ID NO: 13.

FIGS. 2A and 2B show viral replication and cell killing by rVSV-gG versus rVSV-F in Morris (McA-RH7777) rat hepatoma cells in vitro. rVSV-F is a recombinant VSV vector that contains a mutant Newcastle Disease Virus fusogenic glycoprotein gene that was inserted into the 3′-untranslated region of the VSVG gene (Ebert et al., Cancer Res. 64:3265 (2004)). Rat hepatoma cells were infected with rVSV-F or rVSV-gG at MOI=0.01. (A) A TCID₅₀ assay was performed on conditioned media at 0, 3, 6, 10, 24, 48 hours post-infection. (B) An MTT assay for cell viability was performed at 0, 3, 6, 10, 24, 48 hours post-infection. Triplicate samples were analyzed at each time point. Data are shown as the mean+standard deviation (error bars only show+SD).

FIGS. 3A and 3B show inhibition of Natural Killer (NK) cell migration by conditioned media from rVSV-gG, but not rVSV-F, infected rat HCC cells in vitro. (A) Dose response arm NK cell migration in response to rat MIP-1α. The migration assays were performed using 24-well transwell plates. The migration of rat NK cells from the upper chamber to the lower chamber in response to serially diluted rat MIP-1α (0 to about 200 ng/ml) was monitored. (B) Inhibition of NK cell migration in response to MIP-1α by conditioned media from rVSV infected rat HCC cells. The migration assays were performed using 24-well transwell plates. The migration of rat NK cells from the upper chamber to the lower chamber in response to about 10 ng/ml of MIP-1α was monitored in the presence of ultrafiltered and UV-inactivated supernatants from 10⁵ HCC cells infected with rVSV-gG or rVSV-F. Data presented are the mean values of four independent experiments and the results were analyzed statistically by two-sided student t test.

FIG. 4 shows an intratumoral accumulation and distribution of NKR-P1A positive cells after hepatic artery infusion of rVSV-F. Multi-focal HCC-bearing rats were treated with a single injection of rVSV-F, and sacrificed 3 days later. Consecutive sections stained with H&E (upper panels) or immunohistochemistry for NKR-P1A (lower panels) are shown. Tissues were obtained prior to rVSV-F treatment (FIGS. 4Aa and 4Ab) and 3 days after rVSV-F treatment (FIGS. 4Ba and 4Bb) (original magnification, ×10).

FIGS. 5A and 5B show that improved intratumoral rVSV replication and tumor necrosis correlate with depletion of NK cells. Multifocal HCC tumor-bearing Buffalo rats were treated with either anti-asialo GM1 or a control immunoglobulin (Ig), in combination with rVSV-F or PBS administered via hepatic arterial infusion (N=3 for each group). (FIG. 5A) Intratumoral viral titers from tumor cell lysates subjected to TCID₅₀ assays are shown, which are expressed in TCID₅₀ per mg of tumor tissue. Viral titers following treatment with rVSV-F plus control Ig versus rVSV-F plus anti-asialo GM1 were statistically significant by unpaired T-test analysis (p<0.005). (FIG. 5B) Percentage of necrotic areas within tumors, as calculated by morphometric analysis of H&E stained tumor sections are shown. Percentages of necrosis in tumors from animals treated with rVSV-F plus control Ig were compared with those treated with rVSV-F plus anti-asialo GM1 by unpaired T-test (p<0.025).

FIG. 6 shows immunohistochemistry, intratumoral virus titers and tumor necrosis in rVSV-F treated rats in combination with anti-PMN or control rabbit serum. (FIG. 6A) Tissue sections from these same animals were analyzed by immunohistochemical staining for VSVG (FIGS. 6AE and 6AG) and MPO plus cells (FIGS. 6AF and 6AH). (FIG. 6B) Portions of tumor obtained from HCC tumor-hearing Buffalo rats treated with a single injection of rVSV-F at 1.3×10⁷ PFU via hepatic artery, plus either anti-PMN serum or control serum (N=3), were homogenized for plaque assays to determine viral titers (Middle Panel); standard deviations were calculated and data were analyzed by unpaired T-test (p<0.05). (FIG. 6C) In sections of tumors from animals treated with rVSV-F plus anti-PMN or control serum, enhanced tumor necrosis was observed (p<0.05).

FIGS. 7A and 7B show rVSV-gG versus rVSV-F replication in HCC tumors in the livers of immune-competent Buffalo rats. Multi-focal HCC-bearing Buffalo rats were injected through the hepatic artery with PBS (n=3), rVSV-F (n=4), or rVSV-gG (n=4) at 1.3×10⁷ pfu/ml/rat. Tumor samples were obtained from the treated rats at day 3 after virus infusion. Tumor sections were stained with a monoclonal anti-VSVG antibody and counterstained with Hematoxylin (FIG. 7A). Representative sections from rats treated with PBS, rVSV-F, and rVSV-gG are shown in FIGS. 7Aa, 7Ab and 7Ac, respectively (magnification=40×). (FIG. 7B) Intratumoral virus titers were determined by TCID₅₀ assays using tumor extracts on BHK-21 cells. Viral titers are expressed as TCID₅₀/mg tissue (mean+standard deviation). The results were analyzed statistically by two-sided student t test.

FIGS. 8A and 8B show enhanced tumor response in rats treated with rVSV-gG versus those treated with rVSV-F. Multi-focal HCC-bearing Buffalo rats were injected with PBS (n=3), rVSV-F (n=4) or rVSV-gG (n=4) at 1.3×10⁷ pfu/ml/rat and sacrificed 3 days post-virus administration via hepatic artery. (FIG. 8A) 5 mm tumor sections were stained with H&E. Representative sections from rats treated with PBS, rVSV-F and rVSV-gG are shown in frames FIGS. 8Aa, 8Ab, and 8Ac, respectively (magnification=40×). (Figure B) The percentage of necrotic areas in the tumors was measured morphometrically by ImagePro software. Data were shown as mean+standard deviation. The results were analyzed statistically by two-sided student t test.

FIGS. 9A and 9B show immunohistochemical staining and semi-quantification of immune cells in tumors. (FIG. 9A) Representative immunohistochemical sections from tumors and surrounding tissues. Tumor-bearing rats were infused with PBS (FIGS. 9Aa, 9Ad, 9Ag, 9Aj); rVSV-F (FIGS. 9Ab, 9Ae, 9Ah, 9Ak): or rVSV-gG (FIGS. 9Ac, 9Af, 9Ai, 9Al) at 1.3×10⁷ pfu/ml/rat. Samples were obtained from rats at day 3 after virus infusion into the hepatic artery. Sections were stained with mouse monoclonal anti-NKR-P1A (FIGS. 9Aa, 9Ah, 9Ac); polyclonal anti-myeloperoxidase (FIGS. 9Ad, 9Ae, 9Af); monoclonal anti-OX-52 (FIGS. 9Ag, 9Ah, 9Ai); and monoclonal anti-ED-1 (FIGS. 9Aj, 9Ak, 9Al) (magnification=40×). (FIG. 9B) Semi-quantification of immune cells in the lesions after virus treatment: NK cells (FIG. 9Ba), neutrophils (FIG. 9Bb), pan-T cells (FIG. 9Bc), and macrophages (FIG. 9Bd) by ImagePro software. Immune cell index was calculated as ratio of positive cell to unit tumor area (10,000 pixel as one unit tumor area). The results were analyzed statistically by two-sided student t test.

FIG. 10 shows immunofluorescent staining of T and NK cells in tumors. Tumor bearing Buffalo rats were infused with PBS (FIGS. 10a to 10c), rVSV-F (FIGS. 10d to 10f), or rVSV-gG (FIGS. 10g to 10i) at 1.3×10⁷ pfu/ml/rat via the hepatic artery. Samples were obtained at day 3 after virus infusion. Frozen sections were fixed with cold acetone and blocked with 4% goat serum, followed by staining with R-PE-conjugated mouse anti-rat CD3 monoclonal antibody (FIGS. 10a, 10d and 10g) and FITC-conjugated mouse anti-rat NKR-P1A (FIGS. 10b, 10e, and 10h). Merged pictures are shown on FIGS. 10c, 10f, and 10i, respectively (original magnification ×40).

FIG. 11 is a Kaplan-Meier survival curve of multi-focal HCC-bearing Buffalo rats after hepatic arterial infusion of PBS (n=8), rVSV-F (n=10), or rVSV-gG (n=15) at 1.3×10⁷ pfu/ml/rat. Survival was monitored daily and the results were analyzed statistically by log rank test.

FIG. 12 are multicycle growth curves of VSV in rat and human and HCC cells treated with IFN-α. McA-RH7777 (FIG. 12A), Hep3B (FIG. 12B), and HepG2 (FIG. 12C) cells were pre-incubated with various concentrations of rat or human IFN-α overnight and then infected with rVSV-GFP at an MOI of 0.01. Aliquots of tissue culture supernatants were collected at indicated time points and viral genomic RNA was determined by real-time RT-PCR. Results are shown from two independent experiments performed in triplicates (mean±standard deviation).

FIG. 13A shows the molecular structure of mono- and bi-cistronic plasmids: pCMV-Luc is a positive control in which firefly luciferase is under transcriptional control of the CMV promoter. In the bi-cistronic pCMV-EGFP-IRES-Luc plasmids, translation of luciferase is under the control of the preceding IRES, which is from FMDV, HRV2, or EMCV. FIG. 13B shows luciferase expression assay in rat HCC (left panel) and BHK21 (right panel) cells: subcontinent cells in 24 well plates were transfected with Lipofectamine 2000. 24 hrs later, cells were lysed and Luc expression was determined using the Bright-Glo Luciferase system (Promega). The light units per μg protein were plotted against the transfected DNA: FMDV, HRV, and EMCV denote GFP-IRES_{FMDV}-luciferase, GFP-IRES_{HRV2}-luciferase, and GFP-IRES_{EMCV}-luciferase, respectively.

FIG. 14 shows improved intratumoral rVSV replication and tumor necrosis with antibody-mediated depletion of neutrophils and NK cells in tumor-bearing rats. Buffalo rats harboring multi-focal HCC lesions in the liver were intravenously injected with rabbit rat polymorphonuclear leukocytes (PMN) antiserum (Wako; Richmond, Va.); polyclonal rabbit anti-asialo GM1 (Wako Chemical USA, Inc.); or control rabbit IgG at a dose of 1 mg/200 μl/rat at one day before virus infusion through the hepatic artery. A single injection of rVSV-LacZ or rVSV(MΔ51)-LacZ at 5.0×10⁷ pfu/kg was performed on the following day. The antibody injections were repeated at one day post rVSV infusion (n=3 for each group). The treated animals were sacrificed at three days after virus administration and hepatic lesions were collected for neutrophil and NK cell content determination by immunohistochemical staining and morphometric analyses, intratumoral virus titers by TCID₅₀ assays, and tumor necrosis by histological staining followed by morphometric analyses. FIGS. 14A-C, after neutrophil depletion with rabbit anti-rat PMN antiserum; FIGS. 14D-F, after NK cells depletion with rabbit anti-asislo GM1 antiserum. Data are shown as mean+standard deviation. Statistical analyses were performed by the student t-test.

FIGS. 15A-F show viral replication and cell killing by rVSV-LacZ, rVSV(MΔ51)-LacZ, and rVSV(MΔ51)-M3 in rat hepatoma cells in vitro. FIG. 15A is a schematic representation of rVSV-(MΔ51)-LacZ and rVSV(MΔ51)-M3. The full-length pVSV plasmid containing five transcription units, a deletion mutant in matrix protein (MΔ51), and a construct containing the gammaherpesvirus M3 (M3), is shown. The transgenes are preceded by a VSV transcription termination signal, an intergenic region and a transcription start signal (SEQ ID NO: 13), and are inserted into the 3′-untranslated region of the VSVG gene. FIG. 15B is a Western blot using a mono-specific antibody against M3 of conditioned media from cells that were infected with buffer alone, rVSV-LacZ, rVSV(MΔ51)-LacZ, or rVSV(MΔ51)-M3. FIG. 15C shows replication of rVSV-LacZ, rVSV(MΔ51)-LacZ, and rVSV(MΔ51)-M3 in rat HCC cells in vitro at MOI=0.01. CID₅₀ assay was performed on conditioned media at 0, 3, 6, 10, 24, 48, and 72 hours post-infection. FIG. 15D depicts HCC cell killing efficiencies of rVSV-lacZ, rVSV(MΔ51)-lacZ, or rVSV(MΔ51)-M3 in vitro. MTT assays for cell viability were performed at 0, 3, 6, 10, 24, 48, and 72 hours post-infection. Triplicate samples were analyzed at each time point. Data were shown as mean+standard deviation. FIG. 15E is a Western blot using a mono-specific antibody against M3 of tumor extracts from rats at three days after infusion with rVSV(MΔ51)-LacZ or rVSV(MΔ51)-M3. FIG. 15F depicts MCP-1 contents in tumor extracts from rats that were infused with rVSV(MΔ51)-LacZ or rVSV(MΔ51)-M3 as determined by ELISA using a monoclonal antibody to rat MCP-1. In FIGS. 15C, 15D, and 15F, statistical analyses were performed by the student t-test.

FIG. 16 shows immunohistochemical staining of neutrophils and NK cells in tumors of rats treated with rVSV-LacZ, rVSV(MΔ51)-LacZ, or rVSV(MΔ51)-M3. FIG. 16A depicts representative sections of tumor tissues after immunohistochemical staining with an anti-myeloperoxidase antibody that reacts with neutrophils. Tumor-bearing rats were infused with TNE (FIG. 16Aa), 5.0×10⁷ pfu/kg of rVSV-LacZ (FIG. 16Ab), rVSV(MΔ51)-LacZ (FIG. 16Ac), or rVSV(MΔ51)-M3 (FIG. 16Ad), and sacrificed at three days post vector infusion. FIG. 16B depicts semi-quantification of neutrophil contents in the lesions at three days after virus infusion, as quantified by morphometric analysis using the ImagePro software, followed by statistical analyses using two-sided student t-test. FIG. 16C depicts representative sections of tumor tissues after immunohistochemical staining with an NKR-P1A antibody that reacts with rat NK cells. Tumor-bearing rats were infused with TNE (FIG. 16Ca), 5.0×10⁷ pfu/kg of rVSV-LacZ (FIG. 16Cb), rVSV(MΔ51)-LacZ (FIG. 16Cc), or rVSV(MΔ51)-M3 (FIG. 16Cd), and sacrificed at three days post vector infusion. FIG. 16D depicts semi-quantification of NK cell contents in the lesions at three days after virus infusion, as quantified by morphometric analysis using the ImagePro software, followed by statistical analyses using two-sided student t-test.

FIG. 17 is a bar graph showing intratumoral virus replication in rats treated with rVSV-LacZ, rVSV(MΔ51)-LacZ, and rVSV rVSV(MΔ51)-M3. Multi-focal HCC-bearing Buffalo rats were injected with buffer, rVSV-LacZ at its MTD of 5.0×10⁷ pfu/kg, or rVSV(MΔ51)-lacZ and rVSV(MΔ51)-M3 at doses that ranged from 5.0×10⁷ pfu/kg to 5.0×10⁹ pfu/kg. Rats were sacrificed 3 days post-virus administration via the hepatic artery. Virus titers in tumor extracts were determined by TCID₅₀ assays on BHK-21 cells. Viral titers are expressed as TCID₅₀/mg tissue (mean+standard deviation). The results were analyzed statistically by two-sided student t test.

FIG. 18 is a bar graph showing tumor response in rats treated with rVSV-LacZ, rVSV(MΔ51)-LacZ, or rVSV(MΔ51)-M3. Multi-focal HCC-bearing Buffalo rats were injected with buffer, rVSV-LacZ at its MTD of 5.0×10⁷ pfu/kg, or rVSV(MΔ51)-lacZ and rVSV(MΔ51)-M3 at doses that ranged from 5.0×10⁷ pfu/kg to 5.0×10⁹ pfu/kg. Rats were sacrificed 3 days post-virus administration via the hepatic artery. Tumor sections were stained with H&E. Necrosis in tumor was quantified by morphometric analysis using the ImagePro software. Data were shown as mean+standard deviation. The results were analyzed statistically by two-sided student t test.

FIG. 19 shows a Kaplan-Meier survival curve for multi-focal HCC-bearing rats after rVSV-LacZ, rVSV(MΔ51)-LacZ, or rVSV(MΔ51)-M3 treatment. HCC-bearing rats were given hepatic arterial infusion of TNE (open circles, n=6); rVSV-LacZ at its MTD dose of 5.0×10⁷ pfu/kg (solid circles, n=8); rVSV(MΔ51)-LacZ at 5.0×10⁷ pfu/kg (solid squares, n=10), 5.0×10⁸ pfu/kg (solid diamonds, n=10) and 5.0×10⁹ pfu/kg (solid triangles, n=10); and rVSV(MΔ51)-M3 at 5.0×10⁷ pfu/kg (open squares, n=10), 5.0×10⁸ pfu/kg (open diamonds, n=10) and 5.0×10⁹ pfu/kg (open triangles, n=10). Survival was monitored daily and the results were analyzed statistically by the log rank test.

FIG. 20 shows systemic and organ toxicities in tumor-bearing rats after hepatic arterial infusion of rVSV-LacZ, rVSV(MΔ51)-LacZ or rVSV(MΔ51)-M3. Multi-focal HCC-bearing Buffalo rats were injected with buffer, rVSV-LacZ at its MTD dose of 5.0×10⁷ pfu/kg, or rVSV(MΔ51)-lacZ and rVSV(MΔ51)-M3 at doses that ranged from 5.0×10⁷ pfu/kg to 5.0×10⁹ pfu/kg. Blood samples were collected from the left ventricle from the vector treated rats at three days post virus injection, which were then sacrificed for the collection of major organs. FIG. 20a depicts red blood cell and white blood cell contents; FIG. 20b depicts hemoglobin and hematocrits; FIG. 20c depicts serum levels of liver transaminases AST and ALT; FIG. 20d depicts blood urea nitrogen and creatinine contents; FIG. 20e depicts serum TNF-α levels determined by ELISA. Data are shown as mean+standard deviation. The results were analyzed statistically by two-sided student t test. No statistically significant differences were found in all parameters in all treatment groups.

FIG. 21 depicts representative H&E stained sections of the major organs (FIG. 21a, brain; FIG. 21b, spinal cord; FIG. 21c, heart; FIG. 21d, liver; FIG. 21e, lung; FIG. 21f, kidney; FIG. 21g, spleen; FIG. 21h, duodenum) from tumor-bearing rats treated with the highest dose of rVSV(MΔ51)-M3. No tissue pathology was observed.

FIG. 22 is a Kaplan-Meier survival curve for multi-focal HCC-bearing rats after rVSV-EV35, rVSV-UL141, and rVSV-A238L treatment, versus control rVSV-F and PBS. To assess the potential of the recombinant VSV vectors expressing various inflammatory cell suppressive genes as oncolytic agents, rats bearing huge multi-focal HCC tumors in their livers (up to 10 mm in diameter) were randomly assigned to receive either a single infusion of PBS (square, n=8), 1.3×10⁷ pfu of rVSV-EV35 (inverted triangle, n=14), rVSV-UL141 (diamond, n=14), rVSV-A238L (circle, n=15), or an equal dose of the control rVSV-F vector (triangle, n=10) via the hepatic artery. The animals were monitored daily for survival and the results were analyzed statistically by log rank test. While all animals in the PBS (squares, n=8) or rVSV-F treatment groups expired by day 21 or 29, respectively, all groups treated with recombinant VSV vectors expressing various heterologous virus genes that suppress host inflammatory responses resulted in significant prolongation of survival, with some animals achieving survival of 150 days. While treatment with rVSV-EV35, rVSV-UL141, and rVSV-A238L led to significant survival prolongation over the control groups (p<0.0001 vs. rVSV-F and PBS), there was no statistical significance in survival amongst the recombinant VSV vector treatment groups (p>0.2). The long-term surviving rats in these vector treatment groups were sacrificed on day 150 and evaluated for residual malignancy. Macroscopically and histologically, there was no detectable tumor within the liver or elsewhere. These results indicate that huge multi-focal lesions in the liver (up to 10 mm in diameter at the time of oncolytic virus treatment) had undergone complete remission in these animals, which translated into long-term and tumor-free survival.

FIG. 23 is the amino acid sequence of Newcastle Disease Virus fusion protein (SEQ ID NO: 1; GenBank Accession No. CAA50869).

FIG. 24 is the nucleotide sequence encoding the amino acid sequence of Newcastle Disease Virus fusion protein of SEQ ID NO: 1 (SEQ ID NO: 2; GenBank Accession No. X71995).

FIG. 25 is the amino acid sequence of a murine herpesvirus M3 protein (SEQ ID NO: 3; GenBank Accession No. AF127083).

FIG. 26 is the nucleotide sequence encoding the amino acid sequence of murine herpesvirus M3 protein of SEQ ID NO: 3 (SEQ ID NO: 4; GenBank Accession No. AF127083).

FIG. 27 is the amino acid sequence of an equine herpesvirus glycoprotein G (gG_{EHV-1}) (SEQ ID NO: 5; GenBank Accession No. AB187029).

FIG. 28 is the nucleotide sequence encoding the amino acid sequence of an equine herpesvirus glycoprotein G (gG_{EHV-1}) of SEQ ID NO: 5 (SEQ ID NO: 6; GenBank Accession No. AB187029).

FIG. 29 is the amino acid sequence of an Ectromelia virus CKBP 35 kDa chemokine binding protein (SEQ ID NO: 7; GenBank Accession No. AJ277112).

FIG. 30 is the nucleotide sequence encoding the amino acid sequence of an EctroMelia virus CKBP 35 kDa chemokine binding protein of SEQ ID NO: 7 (SEQ ID NO: 8).

FIG. 31 is the amino acid sequence of an African swine fever virus A238L protein (SEQ ID NO: 9; GenBank Accession No. NC_{—}001659).

FIG. 32 is the nucleotide sequence encoding the amino acid sequence of an African swine fever virus A238L protein of SEQ ID NO: 9 (SEQ ID NO: 10).

FIG. 33 is the amino acid sequence of cytomegalovirus Toledo strain UL141 (SEQ ID NO: 11; GenBank Accession No. U33331).

FIG. 34 is the nucleotide sequence encoding the amino acid sequence of cytomegalovirus Toledo strain UL141 SEQ ID NO: 11 (SEQ ID NO: 12).

### DETAILED DESCRIPTION

The present disclosure provides recombinant oncolytic viruses useful for inhibiting the growth, or promoting the killing, of cancerous cells, such as tumor cells. More specifically, the recombinant oncolytic viruses contain a heterologous nucleic acid sequence encoding an inhibitor of inflammatory or innate immune cell migration or function, such as a natural killer cell inhibitor, a chemokine binding protein, or an NF-κB inhibitor. Recombinant oncolytic viruses may, alternatively, contain two or more natural killer cell inhibitor(s), two or more chemokine binding protein(s), and/or two or more NF-κB inhibitor(s).

Thus, this disclosure relates to the unexpected discovery that genetically counteracting host anti-viral inflammatory responses to virus infection (e.g., VSV infection) will substantially enhance intratumoral oncolytic virus replication, oncolysis, and treatment efficacy. Such recombinant oncolytic viruses can be used to treat singular or multi-focal tumors, such as those found in hepatocellular carcinoma (HCC) or other cancers.

Optionally, recombinant oncolytic viruses disclosed herein may also contain one or more heterologous viral internal ribosome entry site (IRES) that is neuronally-silent. This disclosure, therefore, relates further to the surprising discovery that significant attenuation of neuronal VSV replication, without compromising its potency in cancers or tumors, can be achieved through neuron-specific translational control.

Prior to setting forth the disclosure in more detail, it may be helpful to an understanding thereof to set forth definitions of certain terms to be used hereinafter.

### DEFINITIONS

As described herein, any concentration range, percentage range, ratio range or integer range is to be understood to include the value of any integer within the recited range and, when appropriate, fractions thereof (such as one tenth and one hundredth of an integer), unless otherwise indicated. As used herein, “about” or “comprising essentially of” mean±15% of the indicated value or range, unless otherwise indicated. The use of the alternative (e.g., “or”) should be understood to mean either one, both, or any combination thereof of the alternatives. As used herein, the indefinite articles “a” and “an” refer to one or to more than one (i.e., at least one) of the grammatical object of the article. By way of example, “a component” means one component or a plurality of components.

The term “oncolytic virus,” as used herein, refers to a virus capable of selectively replicating in and slowing the growth or inducing the death of a cancerous or hyperproliferative cell, either in vitro or in vivo, while having no or minimal effect on normal cells. Exemplary oncolytic viruses include vesicular stomatitis virus (VSV), Newcastle disease virus (NDV), herpes simplex virus (HSV), reovirus, measles virus, retrovirus, influenza virus, Sinbis virus, vaccinia virus, adenovirus, or the like (see, e.g., Kirn et al., Nat. Med. 7:781 (2001); Coffey et al., Science 282:1332 (1998); Lorence et al., Cancer Res. 54:6017 (1994); and Peng et al., Blood 98:2002 (2001)). The term “oncolytic virus polypeptide,” as used herein, refers to any amino acid sequence encoded by an oncolytic virus genome, which may be required for virus gene expression, replication, propagation, or infection, such as a polymerase (e.g., viral RNA-dependent RNA polymerase or DNA polymerase), a structural protein (e.g., nucleocapsid protein, phosphoprotein, matrix protein, or the like), or a glycoprotein (e.g., envelope).

The term “inflammatory cell inhibitor,” as used herein, refers to a compound or agent capable of reducing the inflammatory effect of cells involved in inflammation or the innate immune response, including inhibiting the effector functions or migration to a target site (e.g., cancerous or tumor cell) of natural killer (NK) cells, neutrophils, monocytes, macrophages, or the like. In this disclosure, the inflammatory cell inhibitor should be understood to mean minimizing the initial innate immune or inflammatory response against a recombinant oncolytic virus. Exemplary inflammatory cell inhibitors include chemokine binding proteins, natural killer cell inhibitors, NF-κB inhibitors, or the like, which may be bacterial, viral, fungal, parasitic or eukaryotic in origin.

The term “chemokine binding protein,” as used herein, refers to any amino acid sequence capable of inhibiting, directly or indirectly, a chemokine from interacting with a receptor or another ligand to modulate an immune response, such as the innate immune or inflammatory response.

The term “Natural Killer cell inhibitor,” as used herein, refers to any amino acid sequence capable of inhibiting or minimizing the function or migration of an NK cell in the innate immune or inflammatory response.

The term “NF-κB inhibitor,” as used herein, refers to any amino acid sequence capable of inhibiting or minimizing the function of NF-κB and, as a consequence, the innate immune or inflammatory response.

As used herein, “inflammation” or “inflammatory response” should be understood to mean a complex set of tissue responses to injury, infection, or other trauma characterized by, for example, altered patterns of blood flow, destruction of damaged or diseased cells, removal of cellular debris, and ultimately healing of damaged tissues.

The term “innate immunity” or “innate immune response” refers to the repertoire of host defenses, both immunological and nonimmunological, that exist prior to or independent of exposure to specific environmental antigens, such as a microorganism or macromolecule, etc. For example, the first host immune response to an antigen involves the innate immune system.

The term “immunogen” or “antigen,” as used herein, refers to an agent that is recognized by the immune system when introduced into a subject and is capable of eliciting an immune response. In certain embodiments, the immune response generated is an innate cellular immune response and the recombinant oncolytic viruses of the instant disclosure are capable of suppressing or reducing the innate cellular immune response.

Immunogens include “surface antigens” that are expressed naturally on the surface of a microorganism (e.g., a virus) or the surface of an infected cell or the surface of a tumor cell.

The term “protective immunity,” as used herein, refers to immunity acquired against a specific immunogen, when a subject has been exposed to the immunogen, which is an immune response (either active/acquired or passive/innate, or both) in the subject that leads to inactivation and/or reduction in the amount of a pathogen and results in immunological memory (e.g., memory T- or B-cells). Protective immunity provided by a vaccine can be in the form of humoral immunity (antibody-mediated) or cellular immunity (T-cell-mediated) or both. For example, protective immunity can result in a reduction in viral or bacterial shedding, a decrease in incidence or duration of infections, reduced acute phase serum protein levels, reduced rectal temperatures, or increase in food uptake or growth.

As used herein, a “vaccine” is a composition that can be used to elicit protective immunity in a recipient. A subject that has been vaccinated with an immunogen will develop an immune response that prevents, delays, or lessens the development or severity of a disease or disorder in the subject exposed to the immunogen, or a related immunogen, as compared to a non-vaccinated subject. Vaccination may, for example, elicit an immune response that eliminates or reduces the number of pathogens or infected cells, or may produce any other clinically measurable alleviation of an infection.

The term “antibody,” as used herein, is intended to include binding fragments thereof which are also specifically reactive with a molecule that comprises, mimics, or cross-reacts with a B-cell or T-cell epitope of a surface molecule or surface polypeptide or other molecule produced by a specific antigen. Antibodies can be fragmented using conventional techniques. For example, F(ab′)₂ fragments can be generated by treating antibody with pepsin. The resulting F(ab′)₂ fragment can be treated to reduce disulfide bridges to produce Fab′ fragments.

The term “therapeutically effective amount” or “effective amount” refers to an amount of a recombinant oncolytic virus composition sufficient to reduce, inhibit, or abrogate tumor cell growth, either in vitro or in a subject (e.g., a dog or a pig or a cow). As noted herein, the reduction, inhibition, or abrogation of tumor cell growth may be the result of necrosis, apoptosis, or an immune response. The amount of a recombinant oncolytic virus composition that is therapeutically effective may vary depending on the particular oncolytic virus used in the composition, the age and condition of the subject being treated, or the extent of tumor formation, and the like.

Recombinant Oncolytic Viruses

By way of background, the successful use of oncolytic viruses to treat cancers may be limited due to their relatively inefficient replication and spread within the solid tumor mass in viva. In addition, the duration of intratumoral replication of oncolytic viruses tends to be limited due to a rapid innate and/or inflammatory anti-viral response that limits the duration of intratumoral replication of the oncolytic viruses, which occurs before the generation of neutralizing anti-viral antibodies in a host. As set forth herein, the present disclosure provides oncolytic viruses having great oncolytic potency (e.g., broad spectrum replication but tumor specific, with replication to high titers) and a short life cycle, which are recombinantly engineered to include nucleic acid sequences that inhibit the anti-viral inflammatory and innate immune responses.

In one aspect, the present disclosure generally pertains to recombinant oncolytic viruses. In one embodiment is provided recombinant oncolytic viruses having a heterologous nucleic acid sequence encoding an inhibitor of inflammatory or innate immune cell migration or function, such as a natural killer cell inhibitor, a chemokine binding protein, an NF-κB inhibitor, or one or more natural killer cell inhibitor(s), chemokine binding protein(s), and/or NF-κB inhibitor(s). Such heterologous nucleic acid sequences can enhance oncolytic potency of the virus by, for example, suppressing anti-viral inflammatory or innate immune responses in a host. In another embodiment, this disclosure provides recombinant oncolytic viruses having a heterologous viral nucleic acid sequence encoding at least one viral internal ribosome entry site (IRES) that is neuronally-silent and operably linked to a nucleic acid sequence that encodes an oncolytic polypeptide. In certain embodiments, an oncolytic virus may be vesicular stomatitis virus (VSV), Newcastle disease virus (NDV), measles virus, influenza virus, sinbis virus, retrovirus, reovirus, herpes simplex virus, vaccinia virus, or adenovirus.

Vesicular Stomatitis Virus (VSV) is an enveloped, non-segmented negative strand RNA virus with inherent tumor selectivity for replication. Rose and Whitt, “Fields Virology” 1221-1242 (D. M. Knipe and P. M. Howley, Philadelphia, Lippincott Williams & Wilkins (2001)). VSV replicates in the cytoplasm of cells, but the cells die within hours after robust viral mRNA and protein synthesis. VSV replicates with great efficiency in most human tumor cells but not in normal cells in vitro, and this difference is even more striking in the presence of IFN-α. Stojdl et al., J. Virol. 74(20):9580-9585 (2000). It has been postulated that this phenomenon is due to the fact that IFN-responsive anti-viral pathways are defective in many tumor cells, including those of human origin. Thus wild-type VSV can replicate within these cells regardless of endogenous IFN production or exogenous IFN treatment. Stojdl et Virol. 74(20):9580-9585 (2000). In contrast, normal cells are fully competent in type I interferon responses, and IFN-mediated inhibition of virus replication in normal cells leads to the selectivity of VSV for tumor cells. In certain embodiments, a recombinant oncolytic virus of this disclosure is administered concurrently or sequentially with interferon, such as type I (e.g., interferon-α) or type II interferon, which may be pegylated-interferon.

Many inflammatory processes are mediated by chemo-attractants and immuno-modulatory molecules called chemokines (Schall and Bacon, Curr. Opin. Immunol. 6:865 (1994)), which play a central role in the host defense against invading microbes and viruses and in the pathogenesis of inflammatory diseases. Rollins, Blood 90:909 (1997) and Baggiolini, Nature 392:565-568 (1998). Chemokines are 8-10 kDa proteins, which interact with G protein-coupled chemokine receptors, and are divided into four structural subfamilies based on the number and arrangement of conserved cysteines: (1) CC chemokines such as RANTES, macrophage inflammatory protein (MIP)-1α and monocyte chemoattractant protein (MCP)-1 are potent attractants for NK, macrophage, immature DC, T- and B-lymphocytes; (2) CXC chemokines such as IL-8 and growth related oncogene (GRO)-α stimulate migration of neutrophils, macrophage, and T- and B-lymphocytes; (3) C chemokine lymphotactin recruits NK and T-lymphocytes; and (4) CX₃C chemokine fractaline recruits neutrophils, NK, and T-lymphocytes. Baggiolini, “The Chemokines” 1-11 (ed. I. Lindley, Plenum, NY (1993); Kelner et al., Science 266:1395 (1994); Schall and Bacon, Curr. Opin. Immunol. 6:865 (1994); and Baggiolini, Nature 392:565-568 (1998). For example, murine gamma herpesvirus-68 M3 (mGHV-M3) is a high-affinity, broad-spectrum secreted vCKBP that binds not only CC and CXC chemokines like equine herpes virus-1 glycoprotein G (gG_{EHV1}), but also binds C and CX3C chemokines responsible for NK, macrophage and T-lymphocyte recruitment. Parry et al., J. Exp. Med. 191:573-578 (2000) and van Berkel et al. Journal of Virology 74(15):6741-6747 (2000).

Successful propagation of viruses within mammalian hosts depends, in part, on their ability to evade the anti-virus arsenal launched by the host immune system and, over the course of evolution, viruses and other organisms have acquired elegant mechanisms to evade immune detection and destruction. Alcami, Nature Immunology 3:36-50 (2003). For example, these mechanisms may include the expression of a natural killer cell inhibitor, a chemokine binding protein (CKBP), an NF-κB inhibitor, or the like.

In some embodiments, the instant disclosure provides a recombinant oncolytic virus comprising a heterologous nucleic acid sequence encoding an inhibitor of inflammatory or innate immune cell migration or function, such as a natural killer cell inhibitor, a chemokine binding protein, an NF-κB inhibitor, or one or more natural killer cell inhibitor(s), chemokine binding protein(s), and/or NF-κB inhibitor(s). In certain embodiments, the heterologous nucleic acid sequence encoded natural killer cell inhibitor is a UL141 polypeptide of human cytomegalovirus (CMV), an M155 polypeptide of murine CMV, or a K5 polypeptide of Kaposi's sarcoma-associated herpes virus. In certain other embodiments, the heterologous nucleic acid sequence encoded chemokine binding protein is an equine herpes virus-1 glycoprotein G (gG_{EHV-1} protein), a murine gamma herpesvirus-68 M3 (mGHV-M3), a Schistosoma mansoni CKBP (smCKBP), a poxvirus CKBP, a myxoma M-T7 CKBP, a human erythroleukemic (HEL) cell CKBP, an orthopoxvirus T1/35 kDa protein, an ectromelia virus (EV) 35 kDa protein (EV35), or the like. For example, the mGHV-M3 is a high-affinity, broad-spectrum secreted vCKBP that binds not only CC and CXC chemokines, as does gG_{EHV1}, but also binds to C and CX3C chemokines responsible for NK, macrophage and T-lymphocyte recruitment. Parry et al., J. Exp. Med. 191:573-578 (2000) and van Berke et al. Journal of Virology 74(15):6741-6747 (2000). In still other embodiments, the heterologous nucleic acid sequence encoded NF-κB inhibitory protein is an A238L protein encoded by African Swine Fever Virus (ASFV). Alternatively, the heterologous nucleic acid sequence that encodes an NF-κB inhibitory protein may be an A52R protein or an N1L protein encoded by a poxvirus; a Vpu accessory protein encoded by human immunodeficiency virus (HIV); or an ORF2 protein encoded by Torque teno virus. In yet further embodiments, the natural killer cell inhibitor, the chemokine binding protein, and/or the NF-κB inhibitor is truncated or lacks a transmembrane domain or is secreted or any combination thereof.

Many cytoplasmic RNA viruses, including VSV, while not normally known to cause central nervous system (CNS) disorders, do exhibit some levels of neural pathology after intravascular administration at high doses in laboratory animals. Schneider-Schnaulies, J. Gen. Virol. 81:1413-1429 (2000). Although VSV has intrinsic tumor specificity due to the attenuated anti-viral responses in many tumor cells, it was noted that when VSV was administered at doses beyond the maximum tolerated dose (MTD), animals showed clinical signs of neural toxicity—such as limb paralysis that occurs in a percentage of the animals treated with VSV at half- to one-log above its MTD (see, e.g., Shinozaki et al., Hepatology 41:196-203 (2005)).

In one aspect, this disclosure provides a recombinant oncolytic viruses, comprising a heterologous viral nucleic acid sequence encoding a viral internal ribosome entry site (IRES) that is neuronally-silent and operably linked to a nucleic acid sequence that encodes an oncolytic polypeptide. The VSV genome has five genes that encode the following oncolytic polypeptides: nucleocapsid protein (VSVN), phosphoprotein (VSVP), matrix protein (VSVM), surface glycoprotein (VSVG), and large subunit of the RNA-dependent RNA polymerase (VSVL, which are all involved in virus replication and/or propagation.

Not wishing to be bound by theory, the VSVG and VSVL proteins have very distinct functions in the life cycle of VSV, and diminished translation of each would have very different but complementary mechanisms in virus attenuation. The G glycoprotein is located in the viral envelope and is responsible for attachment of the virus to the host cell surface to facilitate infection. Carneiro et al., J. Virol. 76:3756-64 (2002). The L polymerase is responsible for transcription of the viral genome into mRNAs for protein synthesis, as well as for replication of the negative-strand viral RNA genome through a full-length intermediate of positive polarity. Barber, Viral Immunology 17(4):516-527 (2004). Therefore, diminished translation of the L polymerase would inhibit the ability of VSV to transcribe its genome into functional mRNAs and replicate its RNA genome, while inhibition of G glycoprotein synthesis would result in the production of “naked” VSV virions without the ability to attach and infect neighboring cells. Due to the role of the L and G proteins for viral gene transcription and replication, as well as infectious virion production and neuronal spread, these were targeted for translational regulation using IRES elements from heterologous viruses that are non-functional in neurons but active in tumor cells, such as HCC cells.

Thus, within certain embodiments, the recombinant oncolytic viruses of this disclosure have a heterologous neuronally-silent viral IRES that is operably linked to a nucleic acid sequence that encodes a VSVN, VSVP, VSVM, VSVG, VSVL, or any combination thereof. In preferred embodiments, the heterologous neuronally-silent viral IRES is operably linked to a nucleic acid sequence that encodes VSVG or VSVL. Oncolytic genes under neuronally-silent IRES-directed translation can attenuate neuro-virulence.

Many viruses have evolved efficient mechanisms to overtake the cellular translational machinery for production of viral proteins while shutting down host mRNA translation. One such mechanism has evolved in the picornaviruses, which share a unique mechanism for translation of their mRNAs. While there are five classes of picornaviruses, their IRES elements can be classified into two major types (type I and II) based on conservation of primary and especially secondary structures. Jackson et al., Trends Biochem. Sci. 15:477 (1990) and Hunt and Jackson, RNA 5:344 (1999). Enterovirus and Rhinovirus contain type I, while Aphthoviruses and Cardioviruses contain type II, IRES elements. These IRES types differ in host protein requirements, as well as in the positions of the initiation codons with regard to their entry sites. Beales et al., J. Virol. 77:6574 (2003). Two picornavirus IRESs that are non-functional in neurons include a human rhinovirus 2 (IRES_{HRV2}) (Gromeier et al., Proc. Natl. Acad. Sci. U.S.A. 93:2370 (1995) and Dobrikova et al., Proc. Natl. Acad. Sci. U.S.A. 100:15125 (2003)) and a foot and mouth disease virus (IRES_{FMDV}). In one embodiment, a recombinant oncolytic virus of this disclosure includes a neuronally-silent picornavirus IRES operably linked to an oncolytic virus polypeptide. In certain embodiments, the virus is a VSV and the IRES is linked to a VSVG glycoprotein or VSVL RNA-dependent RNA polymerase. In other embodiments, the present disclosure provides a recombinant oncolytic virus containing an IRES_{ECMV}, IRES_{HRV2}, IRES_{FMDV}, or any combination thereof. In still another embodiment, the IRES used can be derived from a Hepatitis A virus (HAV), which IRES is classified by itself as a type III IRES—neuronally-silent and hepatically active. In yet another embodiment, the neuronally-silent IRES is IRES_{ECMV}.

In accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al.”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

The terms “polypeptide” and “protein” may be used herein interchangeably to refer to the product (or corresponding synthetic product) encoded by a particular gene, such as a nucleocapsid protein or RNA-dependent RNA polymerase polypeptide. The term “protein” may also refer specifically to the polypeptide as expressed in cells. A “peptide” refers to a polypeptide of ten amino acids or less.

The term “gene” is used herein to refer to a portion of an RNA or DNA molecule that includes a polypeptide coding sequence operatively associated with expression control sequences. Thus, a gene includes both transcribed and untranscribed regions. The transcribed region may include introns, which are spliced out of the mRNA, and 5′- and 3′-untranslated (UTR) sequences along with protein coding sequences. In one embodiment, the gene can be a genomic or partial genomic sequence, in that it contains one or more introns. In another embodiment, the term gene may refer to a complementary DNA (cDNA) molecule (i.e., the coding sequence lacking introns). In yet another embodiment, the term gene may refer to expression control sequences, such as a promoter, an internal ribosome entry site (IRES), or an enhancer sequence.

A “promoter sequence” is an RNA or DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) recognized and bound to by RNA polymerase.

“Sequence-conservative variants” of a polynucleotide sequence are those in which a change of one or more nucleotides in a given codon position results in no alteration in the amino acid encoded at that position.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide.

Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “variant” also includes a polypeptide or enzyme which has at least 60% amino acid identity as determined by BLAST or FASTA algorithms, preferably at least 75%, most preferably at least 85%, and even more preferably at least 90%, and still more preferably at least 95%, and which has the same or substantially similar properties or functions as the native or parent protein or enzyme to which it is compared. The change in amino acid residue can be replacement of an amino acid with one having similar properties (such as, for example, polarity, hydrogen bonding potential, acidic, basic, hydrophobic, aromatic, and the like) or different properties.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.). Reeck et al., Cell 50:667 (1987). Such proteins (and their encoding nucleic acid sequences) have sequence homology, as reflected by their sequence identity, whether in terms of percent identity or similarity, or the presence of specific residues or motifs at conserved positions.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In a specific embodiment, two nucleic acid sequences are “substantially homologous” or “substantially identical” when at least about 80%, and most preferably at least about 90 or at least 95%, of the nucleotides match over the defined length of the nucleic acid sequence, as determined by sequence comparison algorithms, such as BLAST, FASTA, DNA Strider, etc. Exemplary sequences are oncolytic viral species variants that encode similar nucleocapsid, matrix, phosphoprotein, glycoprotein, or polymerase polypeptides. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are “substantially homologous” or “substantially identical” when greater than 80% of the amino acids are identical, or greater than about 90% or 95% are similar (functionally identical). Preferably, the similar or homologous sequences are identified by alignment using, for example, the GCG (Genetics Computer Group, Program Manual for the GCG Package, Version 7, Madison, Wis.) pileup program, or any of the programs described above (BLAST, FASTA, etc.).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al.). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, which are well known variables in the art. The greater the degree of identity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). In certain embodiments, a hybridizable nucleic acid has a length of at least about 10 nucleotides; preferably at least about 15 nucleotides; and more preferably at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

The terms “mutant” and “mutation” mean any detectable change in genetic material, e.g., RNA, DNA, or any process, mechanism, or result of such a change. When compared to a control material, such change may be referred to as an “abnormality”. This includes gene mutations in which the structure (e.g., RNA or DNA sequence) of a gene is altered, any gene or nucleic acid molecule arising from any mutation process, and any expression product (e.g., protein or enzyme) expressed by a modified gene or nucleic acid sequence. The term “variant” may also be used to indicate a modified or altered gene, RNA or DNA sequence, enzyme, cell, etc., i.e., any kind of mutant.

“Amplification” of nucleic acid sequences, as used herein, encompasses the use of polymerase chain reaction (PCR) to increase the concentration of a specific nucleic acid sequence within a mixture of nucleic acid sequences. For a description of PCR, see Saiki et al., Science 239:487 (1988).

“Sequencing” of a nucleic acid includes chemical or enzymatic sequencing. “Chemical sequencing” of DNA denotes methods such as that of Maxam and Gilbert (Maxam-Gilbert sequencing, Maxam and Gilbert, Proc. Natl. Acad. Sci. U.S.A. 74:560 (1977)), in which DNA is randomly cleaved using individual base-specific reactions. “Enzymatic sequencing” of DNA denotes methods such as that of Sanger (Sanger et al., Proc. Nail. Acad. Sci. U.S.A. 74:5463 (1977)), in which a single-stranded DNA is copied and randomly terminated using DNA polymerase, including variations thereof, which are well-known in the art. Preferably, oligonucleotide sequencing is conducted using automatic, computerized equipment in a high-throughput setting, for example, microarray technology, as described herein. Such high-throughput equipment are commercially available, and techniques well known in the art.

A “probe” refers to a nucleic acid or oligonucleotide that forms a hybrid structure with a sequence in a target region due to complementarity of at least one sequence in the probe with a sequence in the target protein.

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of a nucleic acid sequence of interest, or to detect the presence of nucleic acids encoding a polypeptide of interest. In a further embodiment, an oligonucleotide of the invention can form a triple helix with a nucleic acid molecule of interest. In still another embodiment, a library of oligonucleotides arranged on a solid support, such as a silicon wafer or chip, can be used to detect various mutations of interest. Generally, oligonucleotides are prepared synthetically, preferably on a nucleic acid synthesizer. Accordingly, oligonucleotides can be prepared with non-naturally occurring phosphoester analog bonds, such as thioester bonds, etc.

Specific non-limiting examples of synthetic oligonucleotides envisioned for this invention include oligonucleotides that contain phosphorothioates, phosphotriesters, methyl phosphonates, short chain alkyl, or cycloalkyl intersugar linkages or short chain heteroatomic or heterocyclic intersugar linkages. Most preferred are those with CH₂—NH—O—CH₂, CH₂—N(CH)₃—O—CH₂, CH₂—O—N(CH)₃—CH₂, CH₂—N(CH)₃—N(CH)₃—CH₂ and O—N(C H)₃—CH₂—CH₂ backbones (where the phosphodiester is O—PO₂—O—CH₂). U.S. Pat. No. 5,677,437 describes heteroaromatic oligonucleoside linkages. Nitrogen linkers or groups containing nitrogen can also be used to prepare oligonucleotide mimics (U.S. Pat. Nos. 5,792,844 and 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone. Nielsen et al., Science 254:1497 (1991). Other synthetic oligonucleotides may contain substituted sugar moieties comprising one of the following at the 2′ position: OH, SH, SCH₃, F, OCN, O(CH₂)ₙNH₂ or O(CH₂)CH₃ where n is from 1 to about 10; C₁ to C₁₀ lower alkyl, substituted lower alkyl, alkaryl or aralkyl; Cl; Br; CN; CF₃; OCF₃; O—; S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino: polyalkylamino; substituted silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

The terms “vector,” “cloning vector,” and “expression vector” mean the vehicle by which a DNA or RNA sequence (e.g., a heterologous nucleic acid sequence) can be introduced into a host cell to transform the host and promote expression (e.g., transcription and translation) of the introduced sequence. Vectors include plasmids, phages, viruses, etc.

### Formulations and Uses

The recombinant oncolytic virus of this disclosure may be administered in a convenient manner such as by the oral, intravenous, intra-arterial, intra-tumoral, intramuscular, subcutaneous, intranasal, intradermal, or suppository routes or by implantation (e.g., using slow release molecules). Depending on the route of administration of an adjunctive therapy, like an immunotherapeutic agent, the agents contained therein may be required to be coated in a material to protect them from the action of enzymes, acids and other natural conditions which otherwise might inactivate the agents. In order to administer the composition by other than parenteral administration, the agents will be coated by, or administered with, a material to prevent inactivation.

The recombinant oncolytic virus of the present invention may also be administered parenterally or intraperitoneally. Dispersions of the recombinant oncolytic virus component can also be prepared in glycerol, liquid polyethylene glycols, and mixtures thereof and in oils. Under ordinary conditions of storage and use, these preparations may contain a preservative to prevent the growth of microorganisms, such as an antibiotic like gentamycin.

As used herein “pharmaceutically acceptable carrier and/or diluent” includes any and all solvents, dispersion media, coatings, antibacterial and antifungal agents, isotonic and absorption delaying agents and the like. The use of such media and agents for biologically active substances is well known in the art. Supplementary active ingredients, such as antimicrobials, can also be incorporated into the Compositions.

The carrier can be a solvent or dispersion medium containing, for example, water, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be effected by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the recombinant oncolytic viruses of the present disclosure in the required amount of the appropriate solvent with various other ingredients enumerated herein, as required, followed by suitable sterilization means. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum drying and freeze-drying techniques, which yield a powder of the recombinant oncolytic virus plus any additional desired ingredient from a previously sterile-filtered solution thereof.

It may be advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutically or veterinary acceptable carrier.

Pharmaceutical compositions comprising the recombinant oncolytic virus of this disclosure may be manufactured by means of conventional mixing, dissolving, granulating, dragee-making, levigating, emulsifying, encapsulating, entrapping or lyophilizing processes. Pharmaceutical viral compositions may be formulated in conventional manner using one or more physiologically acceptable carriers, diluents, excipients or auxiliaries that facilitate formulating active recombinant oncolytic virus into preparations that can be used biologically or pharmaceutically. The recombinant oncolytic virus compositions can be combined with one or more biologically active agents and may be formulated with a pharmaceutically acceptable carrier, diluent or excipient to generate pharmaceutical or veterinary compositions of the instant disclosure.

Pharmaceutically acceptable carriers, diluents or excipients for therapeutic use are well known in the pharmaceutical art, and are described herein and, for example, in Remington's Pharmaceutical Sciences, Mack Publishing Co. (A. R. Gennaro, ed., 18^{th} Edition (1990)) and in CRC Handbook of Food, Drug, and Cosmetic Excipients, CRC Press LLC (S. C. Smolinski, ed. (1992)). In certain embodiments, recombinant oncolytic virus compositions may be formulated with a pharmaceutically or veterinary-acceptable carrier, diluent or excipient is aqueous, such as water or a mannitol solution (e.g., about 1% to about 20%), hydrophobic solution (e.g., oil or lipid), or a combination thereof (e.g., oil and water emulsions). In certain embodiments, any of the biological or pharmaceutical compositions described herein have a preservative or stabilizer (e.g., an antibiotic) or are sterile.

The biologic or pharmaceutical compositions of the present disclosure can be formulated to allow the recombinant oncolytic virus contained therein to be bioavailable upon administration of the composition to a subject. The level of recombinant oncolytic virus in serum, tumors, and other tissues after administration can be monitored by various well-established techniques, such as antibody-based assays (e.g., ELISA). In certain embodiments, recombinant oncolytic virus compositions are formulated for parenteral administration to a subject in need thereof (e.g., a subject having a tumor), such as a non-human animal or a human. Preferred routes of administration include intravenous, intra-arterial, subcutaneous, intratumoral, or intramuscular.

Proper formulation is dependent upon the route of administration chosen, as is known in the art. For example, systemic formulations are an embodiment that includes those designed for administration by injection, e.g. subcutaneous, intra-arterial, intravenous, intramuscular, intrathecal or intraperitoneal injection, as well as those designed for intratumoral, transdermal, transmucosal, oral, intranasal, or pulmonary administration. In one embodiment, the systemic or intratumoral formulation is sterile. In embodiments for injection, the recombinant oncolytic virus compositions of the instant disclosure may be formulated in aqueous solutions, or in physiologically compatible solutions or buffers such as Hanks's solution, Ringer's solution, mannitol solutions or physiological saline buffer. In certain embodiments, any of the recombinant oncolytic virus compositions described herein may contain formulator agents, such as suspending, stabilizing or dispersing agents. In embodiments for transmucosal administration, penetrants, solubilizers or emollients appropriate to the harrier to be permeated may be used in the formulation. For example, 1-dodecylhexahydro-2H-azepin-2-one (Azon®), oleic acid, propylene glycol, menthol, diethyleneglycol ethoxyglycol monoethyl ether (Transcutol®), polysorbate polyethylenesorbitan monolaurate (Tween®-20), and the drug 7-chloro-1-methyl-5-phenyl-3H-1,4-benzodiazepin-2-one (Diazepam), isopropyl myristate, and other such penetrants, solubilizers or emollients generally known in the art may be used in any of the compositions of the instant disclosure.

Administration can be achieved using a combination of routes, e.g., first administration using an intra-arterial route and subsequent administration via an intravenous or intratumoral route, or any combination thereof.

Methods of Use

In another aspect, the present disclosure provides methods of inhibiting the growth or promoting the killing of a tumor cell or treating cancer, such as hepatocellular carcinoma (HCC), by administering a recombinant oncolytic virus according to the instant disclosure at a multiplicity of infection sufficient to inhibit the growth of a tumor cell or to kill a tumor cell. In certain embodiments, the recombinant oncolytic virus is administered more than once, preferably twice, three times, or up to 10 times. In certain other embodiments, the tumor cell is an HCC cell, which can be treated in vivo, ex vivo, or in vitro.

By way of background, HCC is the third leading cause of death due to cancer and the fifth most common type of cancer in the world, accounting for over one million cases annually. Parkin et al., Bull. World Health Organ. 62(2):163-182 (1984); Murray, Science 274(5288):740-3 (1996); and Parkin et al., Int J Cancer 94:153-156 (2001). HCC arises from the malignant transformation of hepatic parenchymal cells, usually in the setting of chronic liver disease, such as chronic viral hepatitis, alcoholic cirrhosis, hemochromatosis, and autoimmune hepatitis. HCC may present as a solitary tumor or multiple tumors in the liver, and spread outside the liver by invasion into the portal vein or hepatic veins as a malignant thrombus, with distant dissemination to regional lymph nodes, lungs and bones. HCC patients often present with multi-focal lesions in their livers. The liver has a dual blood supply, with the portal vein supplying 75% and the hepatic artery 25% of hepatic blood flow. It is also known that, in humans and animal models, malignant liver tumors have predominantly an arterial blood supply, and hepatic artery infusion is the most commonly employed method for local-regional therapy of HCC in current clinical practice. Mohr et al., Expert Opin. Ilial. Ther. 2:163 (2002). Thus, a therapeutic strategy against HCC should be effective against multi-focal disease. In certain embodiments, treating multi-focal HCC tumors with recombinant oncolytic virus via a vascular route would be advantageous.

Survival of patients with HCC is dependent on the extent of the cancer and underlying liver disease. The prognosis for untreated HCC is poor. For patients with advanced HCC, the prognosis and response to treatment is poor. Treatment modalities for HCC with demonstrated survival prolongation are hepatic resection and local-regional intra-tumoral ablation procedures for solitary tumors, and orthotopic liver transplantation for solitary or multi-focal tumors limited to the liver. But, only a small proportion of patients are candidates for such treatments. Yeung et al., Am. J. Gastroenterol. 100:1995 (2005). Systemic treatment modalities (i.e., chemotherapies such as doxonibicin, 5-fluorouracil α-interferon, and thalidomide) have produced limited responses. Lai et al., Cancer 62:479 (1988); Simonetti et al., Ann. Oncol. 8:117 (1997); and Gastrointestinal Tumor Study Group, Cancer 66(1):135-9 (1990).

Examples of other tumor cells or cancers that may be treated using the methods of this disclosure include breast cancer (e.g., breast cell carcinoma), ovarian cancer (e.g., ovarian cell carcinoma), renal cell carcinoma (RCC), melanoma (e.g., metastatic malignant melanoma), prostate cancer, colon cancer, lung cancer (including small cell lung cancer and non-small cell lung cancer), bone cancer, osteosarcoma, rhabdomyosarcoma, leiomyosarcoma, chondrosarcoma, pancreatic cancer, skin cancer, fibrosarcoma, chronic or acute leukemias including acute lymphocytic leukemia (ALL), adult T-cell leukemia (T-ALL), acute myeloid leukemia, chronic myeloid leukemia, acute lymphoblastic leukemia, chronic lymphocytic leukemia, lymphangiosarcoma, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), Castleman's disease, Kaposi's Sarcoma, hemangiosarcoma, multiple myeloma, Waldenstrom's macroglobulinemia and other B-cell lymphomas, nasopharangeal carcinomas, head or neck cancer, myxosarcoma, liposarcoma, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, cervical carcinoma, vaginal carcinoma, vulvar carcinoma, transitional cell carcinoma, esophageal cancer, malignant gastrinoma, small intestine cancer, cholangiocellular carcinoma, adenocarcinoma, endocrine system cancer, thyroid gland cancer, parathyroid gland cancer, adrenal gland cancer, sarcoma of soft tissue, urethral, penile cancer, testicular cancer, malignant teratoma, solid tumors of childhood, bladder cancer, kidney or ureter cancer, carcinoma of the renal pelvis, malignant meningioma, neoplasm of the central nervous system (CNS), tumor angiogenesis, spinal axis tumor, pituitary adenoma, epidermoid cancer, squamous cell cancer, environmentally induced cancers including those induced by asbestos, e.g., mesothelioma, and combinations of these cancers.

In still another embodiment, the methods involve parenteral administration of a recombinant oncolytic virus, preferably via an artery or via an in-dwelling medical device. As noted above, the recombinant oncolytic virus can be administered with an immunotherapeutic agent or immunomodulator, such as an antibody that binds to a tumor-specific antigen (e.g., chimeric, humanized or human monoclonal antibodies). In another embodiment, the recombinant oncolytic virus treatment may be combined with surgery (e.g., tumor excision), radiation therapy, chemotherapy, or immunotherapy, and can be administered before, during or after a complementary treatment.

In certain embodiments, the recombinant oncolytic virus and immunotherapeutic agent or immunomodulator can be administered concurrently or sequentially in a way that the agent does not interfere with the activity of the virus. In certain embodiments, the recombinant oncolytic virus is administered intra-arterially, intravenously, intraperitoneally, intratumorally, or any combination thereof. In still another embodiment, an interferon, such as interferon-α or pegylated interferon, is administered prior to administering the recombinant oncolytic virus according to the instant invention.

The following non-limiting examples are provided to illustrate various aspects of the present disclosure. All references, patents, patent applications, published patent applications, and the like are incorporated by reference in their entireties herein.

### EXAMPLES

### Example 1

### Construction of Recombinant Oncolytic Viruses

This Example discloses the use of a “reverse genetics” system for the rescue of negative-strand RNA viruses to engineer recombinant VSVs as described herein. Lawson et al., Proc. Natl. Acad. Sci. U.S.A. 92:4477 (1995) and Whelan et al., Proc. Natl. Acad. Sci. U.S.A. 92:8388 (1995).

Plasmid Constructs

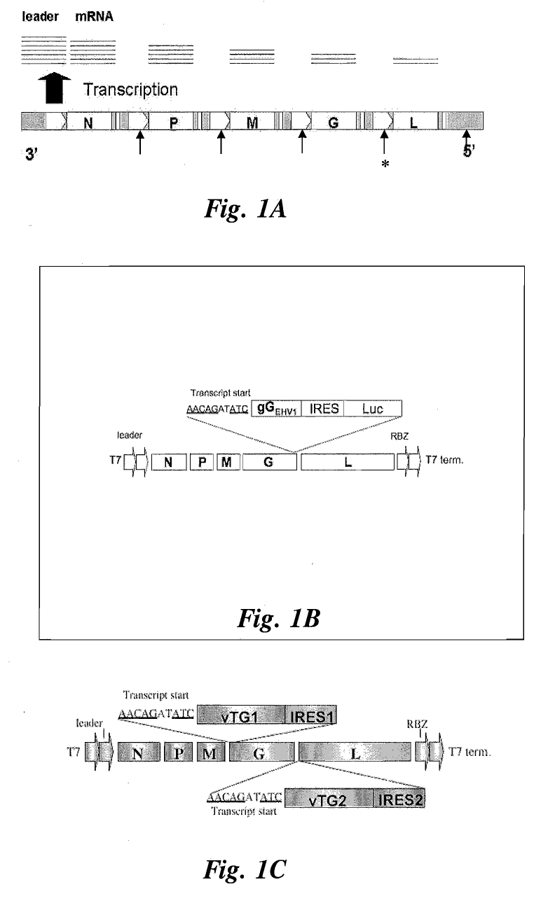

A wild-type VSV (wtVSV) vector (FIG. 1A) was used to generate a recombinant VSV (rVSV) vector encoding a polypeptide capable of inhibiting the migration or activity of inflammatory cells, such as a chemokine binding protein (CBP). Here, equine herpes virus-1 glycoprotein G (gG_{EHV-1}; SEQ ID NOs: 5 and 6; 411 amino acids), an exemplary CBP, was used. A nucleic acid sequence that encodes a secreted form of the glycoprotein G was designed based on a hydrophobicity plot that identified the first 1065 base pairs (bp) of the 1236 by full-length gG_{EHV-1}, coding sequence (see, also, Bryant, et al., EMBO J. 22:833 (2003)). This truncated gG_{EHV-1} nucleic acid sequence was chemically synthesized (GenScript, Piscataway, N.J.) and used to generate a plasmid simultaneously expressing truncated gG_{EHV-1} and a marker protein, firefly Luciferase. The ubiquitously expressed the promiscuous encephalomyocarditis (EMCV) internal ribosome entry site (IRES) was introduced so that these two genes would be expressed as a single transcriptional unit (FIG. 1B). In addition, constructs of an rVSV vector expressing a heterologous viral protein that inhibits NK cell function, such as the UL141 gene from the human cytomegalovirus (UL141_{HCMV}), (Braud et al., Curr Top Microbiol Immunol. 269:117-129 (2002) and Tomasec et al., Nature Immunology 6:181-188 (2005); SEQ ID NOs: 11 and 12), M155 from murine CMV (Lodoen et al., J. Exp Med 200:1075-1081 (2004)), and the K5 gene from Kaposi's Sarcoma-associated Herpesvirus (Orange et al., Nature Immunology 3:1006-1012 (2002)) are made. A genetically modified rVSV vector expressing UL141_{HCMV} was constructed and tested in tumor-bearing animals. Other recombinant oncolytic virus constructs similar to the rVSV described herein can be designed to include more than one heterologous nucleic acid as shown, in one exemplary configuration, in FIG. 1C.

A mutant Newcastle Disease Virus fusion protein, which is based on a 553 amino acid wild-type fusogenic glycoprotein (SEQ ID NOs: 1 and 2) having an L289A mutation, was used to generate an rVSV vector. This construct is referred to as rVSV-F, as previously described by Ebert et al., Cancer Res. 64:3265 (2004).

To generate recombinant VSV with a single methionine deletion at position 51 of the M protein gene (MΔ51), the full-length cDNA VSV clone was digested with XbaI and KpnI and the obtained fragment containing the M protein gene was modified by site-directed PCR mutagenesis (QuikChange II XL; Stratagene; La Jolla, Calif.). Subsequently, the fragment containing MΔ51 was ligated into a similarly digested full-length cDNA clone of VSV encoding the M3 gene constructed as follows.

To create recombinant VSV vectors expressing the secreted form of murine gammaherpesvirus M3 (M3; SEQ ID NOs: 3 and 4), a truncated M3 gene was synthesized chemically in its entirety (GenScript; Piscataway, N.J.). To determine the secreted form, a hydrophobicity plot was generated to predict the C-terminal transmembrane domain. The secreted form of M3 was determined to be the first 1221 by of the full-length gene, which is consistent with the findings of others.

Recombinant Viruses

To rescue the recombinant VSV vector, established methods of reverse genetics were employed. Ebert et al., Cancer Research 63(131:611-613 (2003). BHK-21 cells were infected with a recombinant vaccinia virus that expresses T7 RNA polymerase (vTF-7.3), and then transfected with full length rVSV plasmid in addition to plasmids encoding 17 promoter-driven VSV nucleocapsid (N), phosphoprotein (P), and polymerase (L) using LipofectAMINE 2000 transfection reagent (Invitrogen; Carlsbad, Calif.). BHK-21 cells were also transfected with wtVSV or rVSV. After transfection for 72 hours, supernatants were centrifuged and subjected to ultra-filtration through a 0.22 μm filter followed by plaque purification to completely eliminate vaccinia virus. Titers of rVSV stocks were determined by plaque assays on BHK-21 cells.

Recombinant VSV viruses, designated rVSV-EV35 (ATCC Deposit No. ______), rVSV-UL141-IRES-Luc (ATCC Deposit No. ______), rVSV-gG-IRES-Luc (ATCC Deposit No. ______), rVSV-A238L-IRES-Luc (ATCC Deposit No. ______), rVSV-M3-IRES-Luc (ATCC Deposit No. ______), and rVSV(MΔ51)-M3 (ATCC Deposit No. ______), were deposited with the American Type Culture Collection (ATCC; 10801 University Boulevard, Manassas, Va. 20110-2209, USA) on Dec. 18, 2007. These deposits were made under the provisions of the Budapest Treaty on the International Recognition of the Deposit of Microorganisms for the Purpose of Patent Procedure and the Regulations thereunder (Budapest Treaty). This assures maintenance of a viable culture of the deposit for 30 years from the date of deposit and for at least five (5) years after the most recent request for the furnishing of a sample of the deposit received by the depository. The deposits will be made available by ATCC® under the terms of the Budapest Treaty, and subject to an agreement between Mt. Sinai School of Medicine and ATCC, which assures that all restrictions imposed by the depositor on the availability to the public of the deposited material will be irrevocably removed upon the granting of the pertinent U.S. patent, assures availability of the progeny to one determined by the U.S. Commissioner of Patents and Trademarks to be entitled thereto according to 35 U.S.C. §122 and the Commissioner's rules pursuant thereto (including 37 CFR §1.14 with particular reference to 886 OG 638).

The assignee of the present application has agreed that, if a culture of the materials on deposit should die or be lost or destroyed when cultivated under suitable conditions, the materials will be promptly replaced on notification with another of the same. Availability of the deposited material is not to be construed as a license to practice the invention in contravention of the rights granted under the authority of any government in accordance with its patent laws.

Cell Lines

The rat HCC cell line McA-RH7777 was purchased from the American Type Culture Collection (ATCC) (Manassas, Va.) and maintained in Dulbecco's Modified Eagle Medium (DMEM) (Mediatech; Herndon, Va.) in a humidified atmosphere at 10% CO₂ and 37° C. BHK-21 cells (ATCC) were maintained in DMEM in a humidified atmosphere at 5% CO₂ and 37° C. All culture media were supplemented with 10% heat-inactivated fetal bovine serum (Sigma-Aldrich; St. Louis, Mo.) and 100 U/ml penicillin-streptomycin (Mediatech).

### Example 2

### Characterization of Recombinant Oncolytic Viruses in Tumor Cells in Vitro

This Example discloses that, by the measurement of in vitro replication kinetics and cytotoxicity for rVSV-gG and rVSV-F, recombinant viral vectors that express one or more exogenous genes, as described herein, do not attenuate viral replication in target cells.

Multicycle Growth Curve

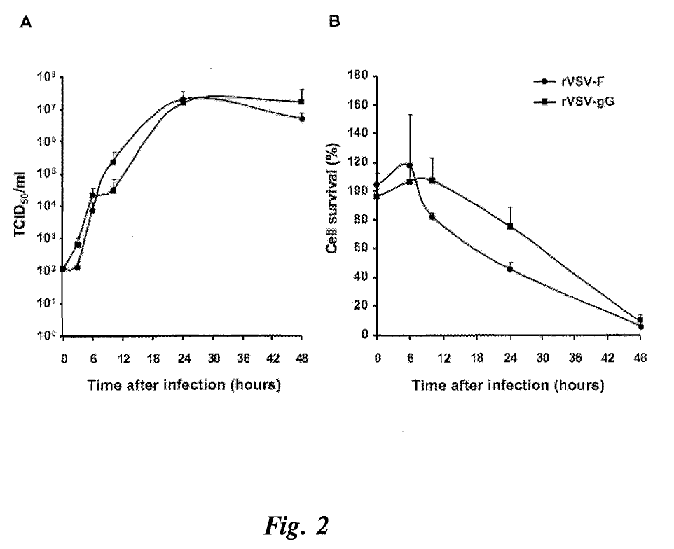

One concern over creating recombinant viral vectors expressing one or more exogenous genes is attenuation of viral replication in target cells. To compare the replication kinetics of rVSV-gG to that of rVSV-F in vitro, the 50% tissue culture inhibitory dose (TCID₅₀) was measured for each construct. Briefly, Morris rat hepatoma cells (McA-RH7777) were plated in 24-well plates at 5×10⁴ cells/well and infected at a multiplicity of infection (MOI) of 0.01 (FIG. 2A). After infection at room temperature for 30 minutes, cells were washed twice with PBS to remove any unabsorbed virus, and fresh complete medium (Dulbecco's Modified Eagle Medium (Mediatech, Herndon, Va.) supplemented with 10% heat-inactivated fetal bovine serum (Sigma-Aldrich, St. Louis, Mo.) and 1000/ml penicillin-streptomycin (Mediatech)) was added. At the indicated time points after infection, 100 μl of supernatant was collected and assayed for viral titer by TCID₅₀ assays.

By 48 hours post-infection, both vectors replicated to similar titers, indicating that the new recombinant vector introduced no significant changes to the viral life cycle or viral yield of VSV in rat HCC cells in vitro.

In Vitro Cytotoxicity

McA-RH7777 cells were seeded in 24-well plates at 5×10⁴ cells/well overnight. The following day, cells were either mock infected or infected with rVSV-F or rVSV-gG at an MOI of 0.01. Cell viability was measured on triplicate wells at the indicated time points after infection using the 3-(4,5-dimethylthiazol-2-yl)-2,5-diphenyltetrazolium bromide (MTT) assay (Cell Proliferation Kit I; Roche; Indianapolis, Ind.). All cell viability data are expressed as a percentage of viable cells as compared to mock-infected controls at each time point.

Although the rVSV-gG infected cells were slightly delayed in cell killing as compared to rVSV-F at 24 hours post-infection, both viruses caused nearly 100% cell death by 48 hours post-infection (FIG. 2B). These results show that rVSV-gG_{EHV-1}, is able to effectively kill Morris hepatoma cells in vitro.

### Example 3

### Inhibition of Natural Killer (NK) Cell Migration In Vitro by a Recombinant Oncolytic Virus Expressing a CKBP Gene (RVSV-CKBP)

This Example demonstrates, thru migration assays of NK cells in response to the CC chemokine macrophage inflammatory protein-1α (MIP-1α), that the gG_{EHV-1}, protein is functional when expressed by rVSV-gG infected cells.

Male buffalo rat were injected intraperitoneally (i.p.) with 10 μg/g of Poly I:C (EMD biosciences; La Jolla, Calif.) and then sacrificed 24 h later. The mononuclear cells (MNCs) in splenocytes were prepared by crushing the spleens, followed by gradient centrifugation in Lymphocyte Cell Separation Media (Cedarlane; Ontario, Canada). NK cells were enriched from the MNCs by Miltenyi magnetic separation after binding the cells with phycoerythrin (PE)-conjugated anti-rat CD161a antibody (10/78, BD Biosciences; San Diego, Calif.), followed by anti-PE MicroBeads (Miltenyi Biotec; Auburn, Calif.), according to the manufacturer's instructions. Analysis by flow cytometry showed that the preparations were greater than 85% pure. The purified cells were cultured in complete DMEM medium containing 0.5% bovine serum albumin (BSA, Sigma). McA-RH7777 cells in serum-free DMEM (Mediatech, Herndon, Va.) were infected with rVSV-F or rVSV-gG_{EHV-1}, at an MOI of 5. The culture media was harvested 24 hour later and filtered through 0.2 μm Acrodisc syringe filters (Pall Corp., Ann Arbor, Mich.) after infectious virus in the filtrate was quantitatively inactivated by UV irradiation. To determine the optimal concentration of rat MIP-1α for the migration of rat NK cells, a dose response was conducted in 24-well transwell plates (Corning, INC; Corning, N.Y.) with 5 μm pore size filters using 0, 25, 50, 100, or 200 ng/ml rat MIP-1α (Peprotech; Rocky Hill, N.J.) in the lower chambers following a 4 hour incubation at 37° C. Migration of rat NK cells (5×10⁵/well) from the upper to lower chamber in response to the varying concentrations of chemokine was monitored. At the defined dose of MIP-1α (100 ng/ml), migration of rat NK cells was measured in the presence of filtered and UV inactivated culture supernatants from McA-RH7777 cells infected with either rVSV-gG or rVSV-F (MOI=5, 24 h).

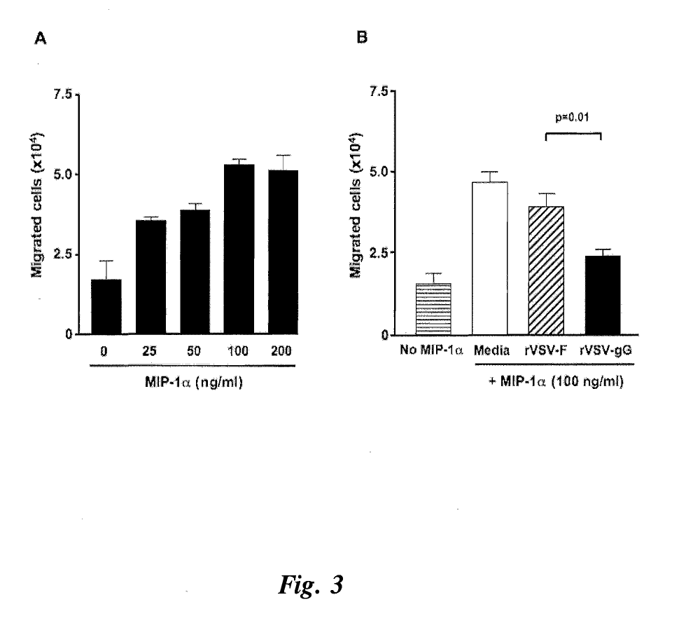

The migration of rat NK cells from the upper to the lower chamber increased in a MIP-1α dose-responsive manner until saturation was reached at around 100 ng/ml (FIG. 3A). This dose of MIP-1α was then used to evaluate the inhibition of rat NK cell migration by conditioned media from rVSV-gG versus rVSV-F infected rat McA-RH7777 hepatoma cells, which were ultra-filtered and UV-irradiated to quantitatively remove infectious viruses. As controls, media from mock-infected cells and migration in the absence of MIP-1α were used. The results show that the number of migrating NK cells was significantly inhibited by conditioned media from rVSV-gG infected rat HCC cells as compared to that from rVSV-F (FIG. 3B, p=0.01).

### Example 4

### Enhancement of Oncolytic Potency by Depleting NK Cells in the Host

This Example demonstrates the enhancement of oncolytic potency thru the inhibition of NK cell function.

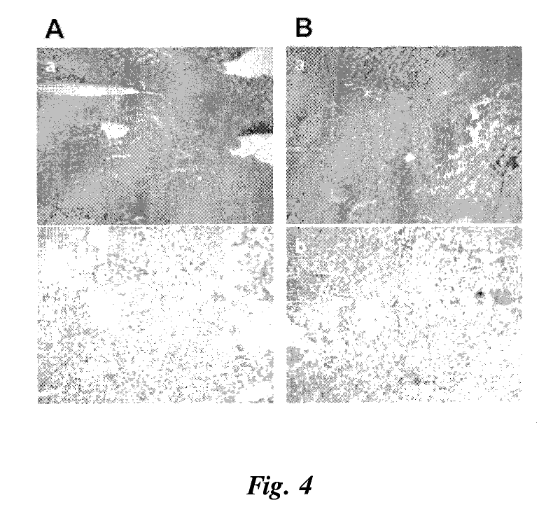

NK cells play an important role in anti-viral immunity (Hamerman et al., Current Opinion in Immunology, 17:29-35 (2005)) and are abundant in the normal liver, accounting for approximately one-third of intrahepatic lymphocytes. Chen et al., J. Virol Hepatitis 12:38-45 (2005). NK cells exert their anti-viral functions through their own natural cytotoxicity, as well as through the production of cytokines. Chen et al., J. Virol Hepatitis 12:38-45 (2005) and Hamerman et al, Current Opinion in Immunology 17:29-35 (2005). To evaluate the distribution of NK cells within the hepatic tumors treated with rVSV-F, multi-focal HCC bearing rats were sacrificed before or 3 days after hepatic artery infusion of rVSV-F at its MTD and frozen liver and tumor tissues were obtained. Immunohistochemical staining for NK cells was performed using the NKR-P1A antibody, and H&E staining was performed on consecutive sections to determine the locations of these immune cells. Tumor-bearing rats treated with rVSV-F had much greater infiltration of NKR-P1A positive cells into the solid tumor mass (FIG. 4B), while untreated animals showed only few NKR-P1A positive cells within the tumors and along the peri-tumoral regions (FIG. 4A).

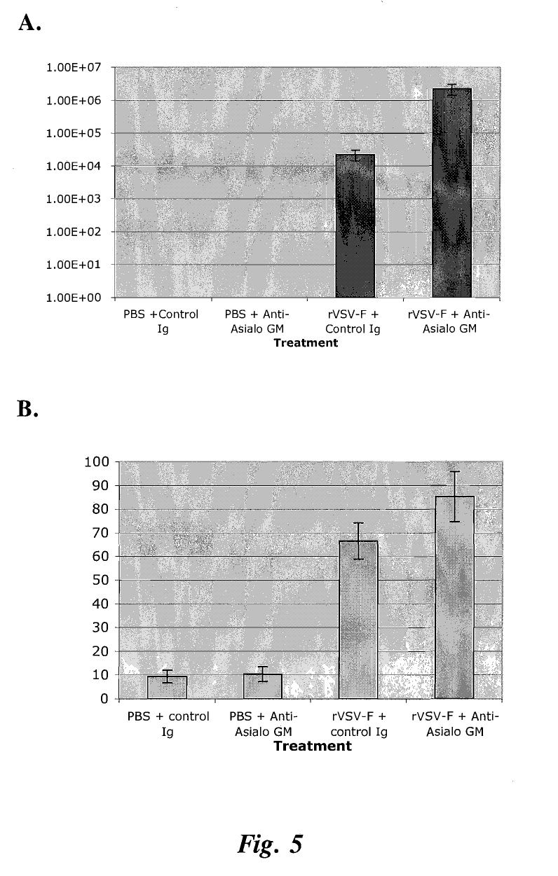

To test the effect of NK cell depletion on VSV replication in HCC tumors, we utilized rabbit anti-asialo GM1, which has activity against mouse and rat NK cells, versus a control rabbit 1 g. Using a reported safe and effective dose of 1 mg per rat (Lin et al., Transplantation 64(12):1677-83 (1997)), Buffalo rats harboring multi-focal HCC lesions in the liver were treated with a single hepatic arterial injection of rVSV-F at its MTD in the presence of the NK-depleting or control antibodies, and the treated animals were sacrificed after 3 days. Lysates from frozen tumor tissues were subjected to TCID₅₀ assays, and enhanced viral titers by 2-logs were observed in tumors harvested from animals treated with the NK-depleting antibody over that of control (FIG. 5A). Liver sections containing tumors were obtained for histological staining, and the percentage of necrosis within tumors was calculated by morphometric analysis, which revealed an enhancement of tumor necrosis with NK cell depletion (FIG. 5B). Taken together, the results suggest that a depletion of NK cells has substantially elevated intratumoral VSV replication, which then led to enhanced oncolysis and tumor response.

### Example 5

### Enhancement of Oncolytic Potency by Depleting PMN Cells in the Host

This Example demonstrates the enhancement of oncolytic potency of rVSV, and corresponding enhanced tumor response, by chemokine binding protein mediated depletion of PMNs can substantially elevate the oncolyic potency of rVSV.

A polyclonal rabbit antibody (Cedarlane Laboratories, Ltd.) against rat polymorphonuclear leukocytes (PMNs) was used to determine the effect of PMN-depletion on. VSV replication in HCC tumors. A dose response study was conducted in normal Buffalo rats to determine that a safe and effective dose of anti-PMN antiserum was 50 μl/rat (results not shown). Using this defined dose Buffalo rats harboring multi-focal HCC lesions were treated with a single hepatic arterial injection of rVSV-F in the presence of the PMN-depleting or control antibodies, and sacrificed on day 3 after rVSV-F treatment. Tumor-containing liver tissue sections were obtained for immunohistochemical staining for MPO⁺ cells and VSVG.

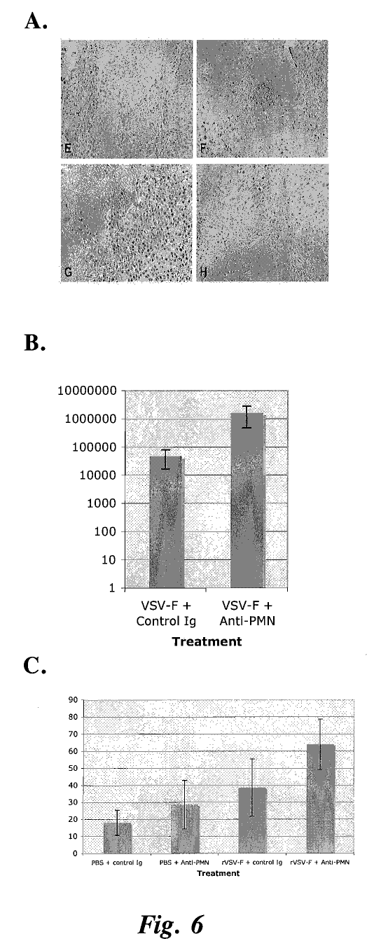

There was a significant reduction of MPO⁺ cells in the tumors, while there were more VSVG staining (FIG. 6A). Intratumoral virus titers were determined by plaque assay of tumor lysates and were shown to be elevated by 1.5-logs in the presence of the PMN-depleting antibody (FIG. 6B). There was also a statistically significant enhancement of necrotic areas in the lesions after anti-PMN treatment (FIG. 6C).

Taken together, these results suggest that depletion of PMN's can substantially elevate the oncolyic potency of rVSV that can lead to enhanced tumor response.

### Example 6

### Elevated Replication of rVSV-CKBP in Multi-Focal HCC Tumors

This Example demonstrates that rVSV-gG is capable of enhanced replication as compared to rVSV-F in multi-focal HCC tumors.

Multi-focal HCC lesions were elicited in a rat model to assess the in vivo effect of vector-mediated gG_{EHV-1} production on oncolysis and viral replication within tumors. Six-week old male Buffalo rats were purchased from Harlan (Indianapolis. IN) and housed in a specific pathogen-free environment under standard conditions. To establish multifocal HCC lesions within the liver, about 10⁷ syngeneic McA-RH7777 rat hepatoma cells (in a 1 ml suspension of DMEM) were infused into the portal vein. Shinozaki et al., Mol. Ther. 9(3):368-76 (2004). Multi-focal lesions of HCC that ranged in size from about 1 mm to about 10 mm in diameter developed in the rat livers by 21 days post-implantation. The tumor-bearing rats were treated with phosphate buffered saline (PBS, control), 1.3×10⁷ plaque forming units (pfu) of rVSV-gG, or 1.3×10⁷ pfu of rVSV-F, in a 1 ml dose via hepatic artery infusion.

To evaluate tumor response to viral treatment, animals were sacrificed 3 days after infusion and tumors were subjected to histological, immunohistochemical and immunofluorescent staining, as well as snap-frozen for intratumoral viral titer quantification via TCID₅₀ analysis. In addition, groups of animals infused with VSV vectors or PBS control were followed for survival, which was monitored daily in all animals. For comparison of individual data points, two-sided student t-test was applied to determine statistical significance.

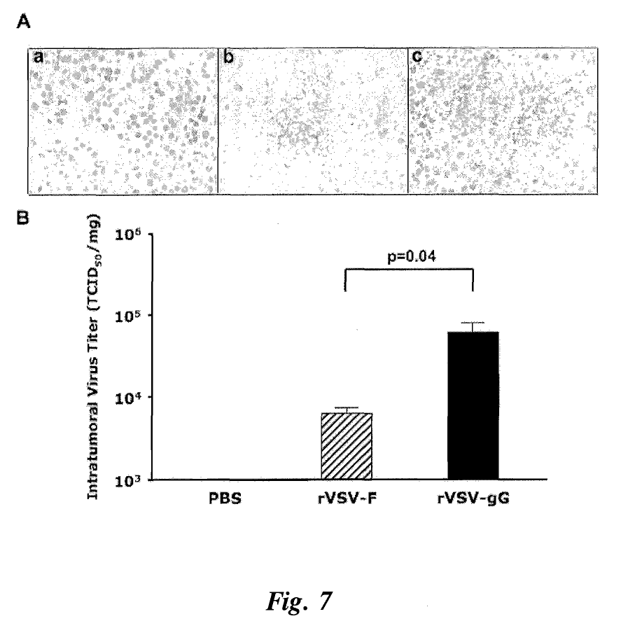

Immunohistochemical staining using a monoclonal anti-VSVG antibody revealed the presence of VSVG within tumors of rVSV-gG_{EHV-1} treated animals, which was more abundant than that observed in the rVSV-F treated rats (FIG. 7A). To quantify the virus yields in the lesions, lysates prepared from snap-frozen tumor samples from each animal were subjected to TCID₅₀ analysis. While rVSV-F infusion resulted in titers less than 10⁴ TCID₅₀/mg of tumor tissue, rVSV-gG_{EHV-1} replicated within tumors to yield titers of one-log higher at 10⁵ TCID₅₀/mg of tumor tissue (FIG. 7B, p=0.04).

### Example 7

### Enhanced Oncolytic Effect of rVSV-CKBP on Multi-Focal HCC Tumors

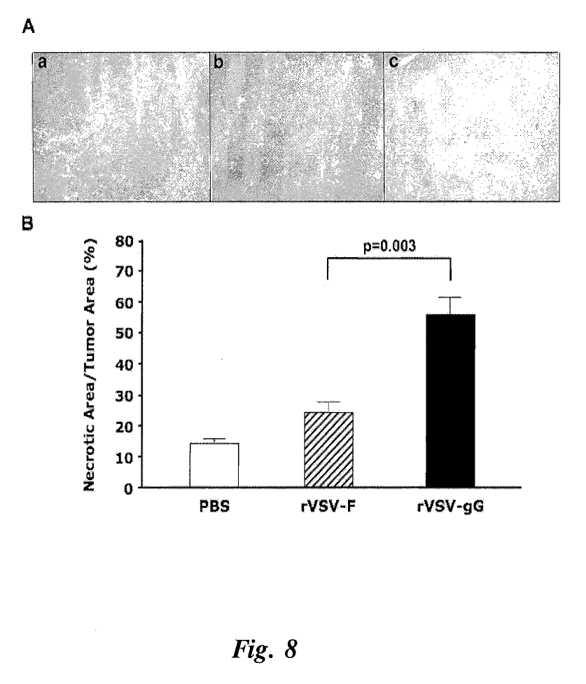

To determine the impact of enhanced intratumoral replication of the rVSV-gG_{EHV-1} vector on tumor viability, tumor-containing liver sections from Example 6 were examined by H&E staining (FIG. 8A—(a) PBS, (b) rVSV-F, (c) rVSV-gG_{EHV-1}) and analyzed morphometrically for determination of percentage of necrosis (FIG. 8B). Liver samples containing tumor were fixed overnight in 4% paraformaldehyde and then paraffin-embedded. Thin sections were subjected to either H&E staining for histological analysis or immunohistochemical staining using monoclonal antibodies against VSVG protein (Alpha Diagnostic, TX) or myeloperoxidase (MPO) (Abcam, MA). Another set of liver samples containing tumor were fixed overnight 4% paraformaldehyde and then equilibrated in 20% sucrose in PBS overnight. Frozen sections were subjected to immunohistochemical staining using monoclonal antibodies against NKR-P1A (BD Pharmingen, CA), OX-52 (BD Pharmingen, CA), or ED-1 (Chemicon, CA). Semi-quantification of positively stained cells was performed using ImagePro Software (Media Cybernetics, Inc., Silver Spring, Md.), and immune cell index was calculated as a ratio of positive cell to unit tumor area (10,000 pixels as one unit tumor area). Frozen sections were fixed with cold acetone and blocked with 4% goat serum, followed by staining with R-PE-conjugated mouse anti-rat CD3 monoclonal antibody (BD Pharmingen, CA) and FITC-conjugated mouse anti-rat NKR-P1A antibody (BD Pharmingen, CA). Nuclear DNA was stained with 4′,6′-diamidino-2-phenylindole (DAPI). Coverslips were mounted on glass slides using VECTASHIELD Mounting Medium (Vector Laboratories, CA).

Using ImagePro software, necrotic areas were measured and represented as a percentage of the entire tumor area. Tumors within the rVSV-gG treatment group after 3-days were approximately 55% necrotic, which represents a significant increase over the rVSV-F treatment group of approximately 25% necrosis (FIG. 8B, p=0.003). In the PBS control group, less than 15% necrosis was observed, which was caused by spontaneous necrosis that occurs in this tumor type in viva. To examine the safety of enhanced oncolytic virus potency, histopathological sections were carefully examined at the border region between tumor and liver tissues and neighboring liver parenchyma. The surrounding liver histology was found to be completely normal, with no evidence of pathology (results not shown).

### Example 8

### Reduced Inflammatory Cells in Multi-Focal HCC Tumors after rVSV-CKBP Treatment

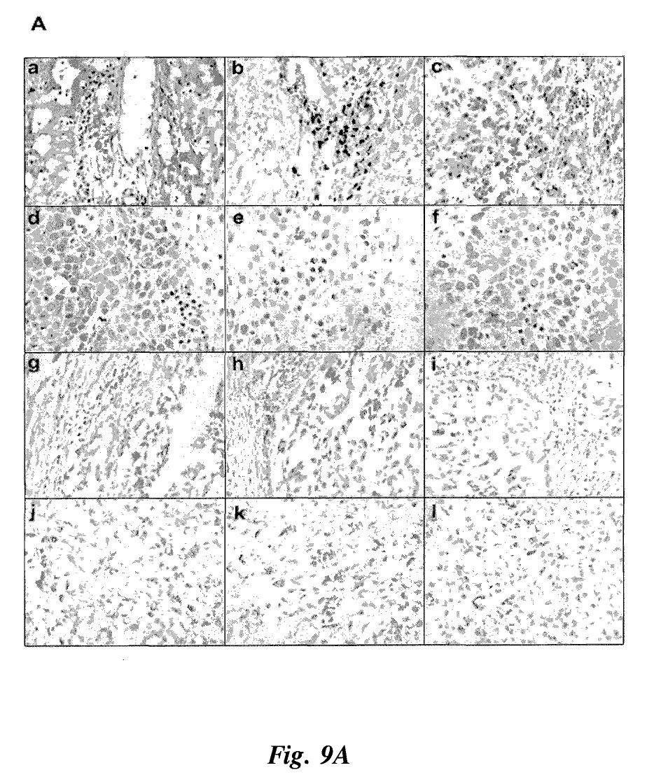

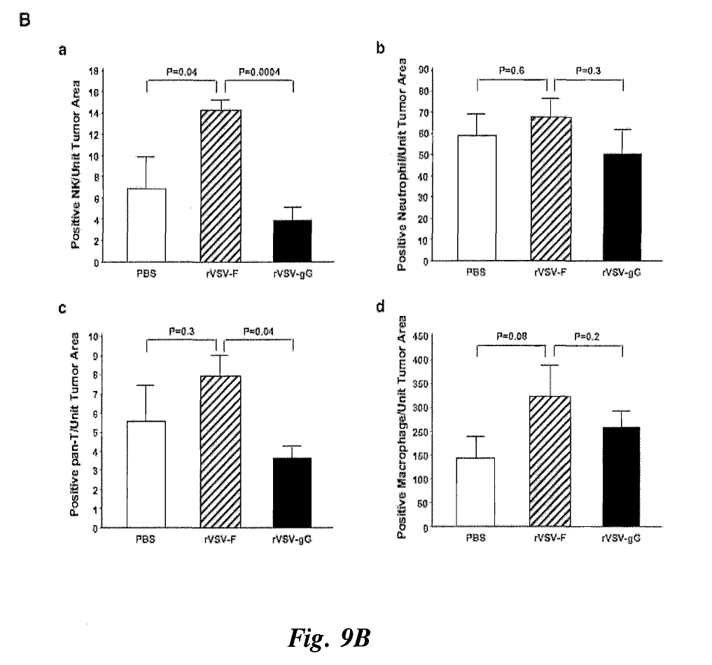

Tumor-containing liver sections from Example 6 were examined for immunohistochemical staining of various immune cell types (FIG. 9A). Sections were stained for NK cells with anti-NKR-P1A (Frames a-c), neutrophils by anti-myloperoxidase (Frames d-f), pan-T cells by anti-OX-52 (Frames g-i), and macrophages by anti-ED-1 (Frames j-l). Semi-quantification of marker-positive cells, using ImagePro software, revealed that there was substantial accumulation of NK cells at the lesions after rVSV-F infusion over the PBS treated rats (FIG. 9Ba, p=0.04), which was substantially reduced after rVSV-gG treatment (FIG. 9Ba, p=0.0004). While there was no statistically significant difference in neutrophil (p=0.3) or macrophage content (p=0.2) within tumors of rats treated with the two rVSV vectors (FIG. 9Bb), there was a statistically significant difference in the number of pan-T marker-positive cells (FIG. 9Bc, p=0.04).

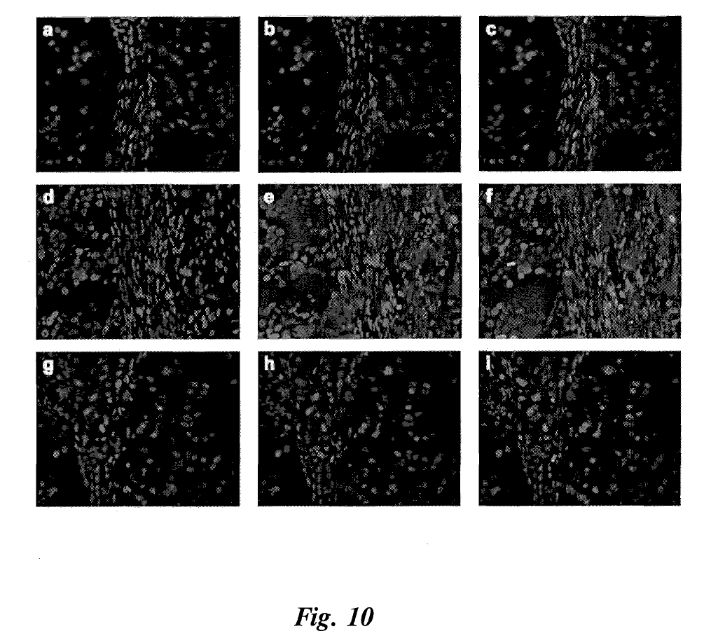

To determine whether these were NKT cells or T-lymphocytes, indirect immunofluorescent staining was performed. Consecutive tumor sections from PBS, rVSV-gG or rVSV-F treated animals were stained with R-PE-conjugated mouse anti-rat CD3 antibody and FITC-conjugated mouse anti-rat NKR-P1A antibody (FIG. 10). Merged pictures indicate that the pan-T-positive cells present in the tumors after rVSV-F treatment are NKT cells rather than T-lymphocytes. Collectively, the results indicate that NK and NKT cells might be the effector inflammatory cells, and their chemotaxis to the tumor sites was inhibited by vector-mediated expression of gG_{EHV-1}.

### Example 9

### Efficacy of rVSV-CKBP in Multi-Focal HCC Tumors

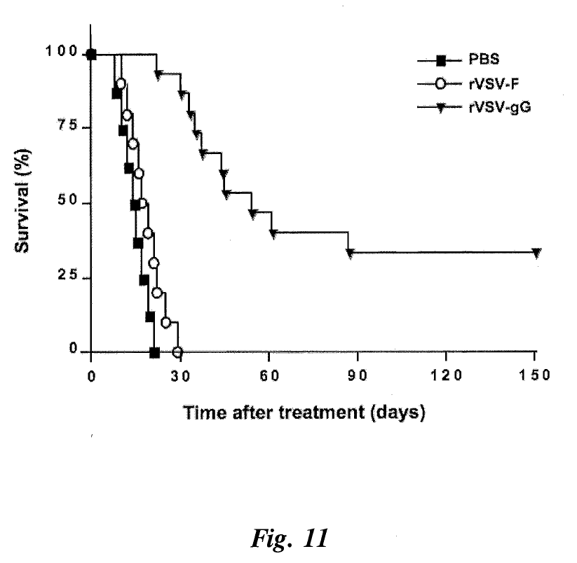

To assess the potential of the rVSV vector expressing a cytokine binding protein that inhibits NK and NKT cell chemotaxis, rats bearing multi-focal HCC tumors in their livers were randomly assigned to receive either a single infusion of PBS (n=8), 1.3×10⁷ pfu of rVSV-gG_{EHV-1} (n=15), or an equal dose of the control rVSV-F vector (n=10) via the hepatic artery, and animals were monitored daily for survival (FIG. 11). Survival curves of animals were plotted according to the Kaplan-Meier method, and statistical significance in different treatment groups was compared using the log-rank test. Results and graphs were obtained using the GraphPad Prism 3.0 program (GraphPad Software, San Diego, Calif.).

While all animals in the PBS or rVSV-F treatment groups expired by day 21 or 29, respectively, rVSV-gG treatment resulted in a highly significant prolongation of survival (P=0.00001), with 5 of 15 animals (33%) achieving long-term survival of 150 days. Furthermore, the long-term surviving rats in the rVSV-gG treatment group were sacrificed on day 150 and evaluated for residual malignancy. Macroscopically, there was no visible tumor within the liver or elsewhere, and there was no histological evidence of residual tumor cells or hepatitis. These results indicate that even large multi-focal lesions (up to 10 mm in diameter at the time of treatment) had undergone complete remission in these animals, which translated into long-term and tumor-free survival.

### Example 10

### Prophylactic Treatment with Interferon-α

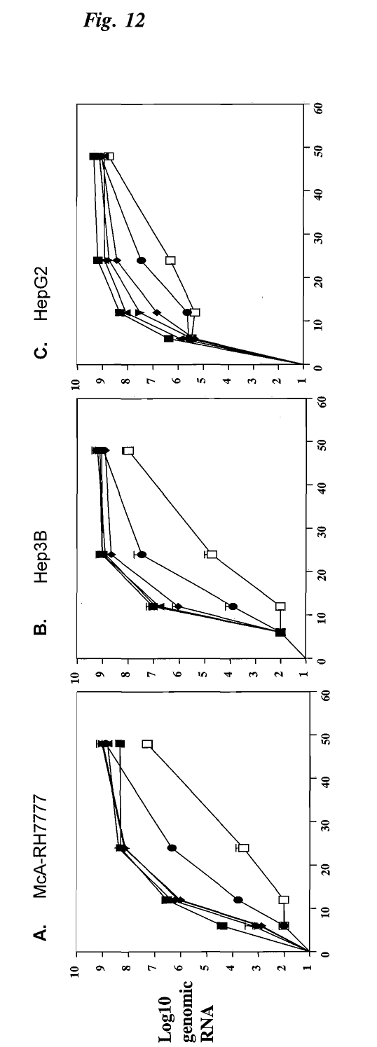

To elevate the maximum tolerated dose (MTD) without sacrificing efficacy, prophylactic treatment with interferon-α was used before administering rVSV vectors, such that viral replication in the normal neurons would be significantly attenuated while intratumoral virus replication will not be affected due to tumor cell's attenuated responses to IFN's. To evaluate the replication potential of VSV in the presence of various concentrations of IFN-α in HCC cells in vitro, rat (McA-RH7777) and human (Hep3B and HepG2) HCC cell lines were pre-incubated with rat and human IFN-α, respectively, overnight and then infected with rVSV-GFP at an MOI of 0.01. The supernatants were harvested at various time points post infection, total RNAs from the cell culture supernatants were prepared, and the RNA samples were analyzed for the presence and concentration of genomic VSV RNA by real-time RT-PCR (FIG. 12).

The results show that VSV replication in rat and human HCC cells was not attenuated in the presence of rat and human IFN-α, respectively, at concentrations of up to about 10 IU/ml. The replication kinetics of VSV in HCC cells pre-incubated with 100 IU/ml IFN-α appeared to be slightly delayed but reached similar titers at 48 h after infection. At 1000 IU/ml rat and human IFN-α VSV replication was significantly attenuated in rat McA-RH7777 and human Hep3B cells, while the virus could still replicate to high levels in human HepG2 cells, indicating that the latter cell line was more unresponsive to the anti-viral activity of human IFN-α. Therefore, VSV appeared to retain its replication potential in rat and human HCC cells in vitro after pre-incubation with relatively high doses of rat and human IFN-α, respectively.

### Example 11

### Heterologous IRES Activity in a Recombinant Oncolytic Virus

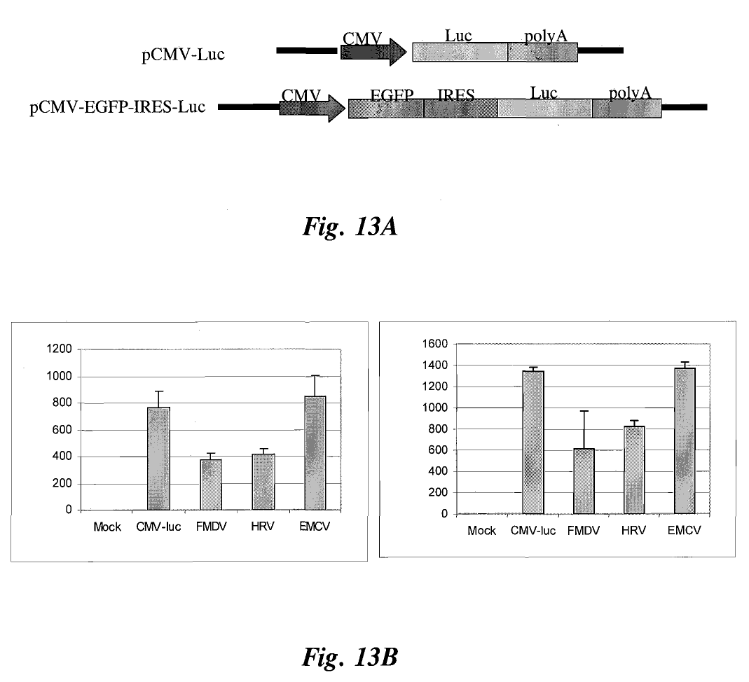

To show that IRES_{HRV2} and IRES_{FMDV} do function in HCC cells, plasmids containing expression cassettes of CMV promoter driven GFP-IRES_{HRV2}-luciferase and GFP-IRES_{FMDV}-luciferase were constructed (FIG. 13A) and used to transfect HCC and BHK-21 cells in vitro, followed by quantification of luciferase activities in cellular extracts. A similar construct containing a promiscuous type II IRES element from the promiscuous encephalomyocarditis Virus (EMCV) as well as one without an intervening IRES element (FIG. 10A) were also tested. While the IRES_{EMCV} driven luciferase construct was as effective as the one without an intervening IRES element, approximately 50-60% of luciferase activities were obtained from the IRES_{HRV2} and IRES_{FMDV} constructs in both cell types (FIG. 13B).

These results indicate that the two type I IRES elements are functional in HCC cells, and that the IRES-containing VSV vectors can be rescued in BHK-21 cells.

### Example 12

### Efficacy of Repeated rVSV Intra-Arterial Delivery

To assess the anti-tumor efficacy of the single and double IRES-containing rVSV-F vectors, multi-focal orthotopic HCC tumors is generated by the previously established method of infusion of 1×10⁷ rat HCC cells (McA-RH7777) into the portal vein of syngeneic Buffalo rats. Huge multi-focal lesions of HCC is developed in the livers of these rats after 21 days. In this study, animals will be randomized to receive 3-time injections at days 0, 2 and 4 via an indwelling catheter in the hepatic artery of the single and double IRES-containing rVSV vectors, the parental rVSV vector, or UV-inactivated virus. The doses of the single and double IRES-containing vectors will vary from their respective MTDs to two logs below in half-log decrements in order to determine their minimum effective doses, which may or may not equate to their respective MTDs. The experimental endpoint will be survival and there will be a minimum of 15 animals per treatment group to allow statistical analysis of the results. Animal survival will be analyzed by the Kaplan-Meier method and statistical analyses of the survival curves of different groups will be made by the log-rank test. In addition, to determine if the two IRESs have synergistic or additive effects in attenuation of neural toxicity, a proportional hazards model containing an interaction term will be used. Indicator variables will be coded as x=0 or 1 depending on the presence or absence for IRES-1, y=0 or 1 depending on the presence or absence for IRES-2 and z=xy representing the interaction. All surviving animals will be sacrificed after 120 days and the major organs will be examined histologically. Statistical non-significance of the interaction term would indicate that the two IRESs are additive in their effect. Statistical significance of the interaction term would indicate that the two IRESs have a synergistic effect. Slud, Biometrics 50:25-38 (1994).

To determine the rate of intratumoral virus replication and tumor response, additional animal treatment groups at the respective minimum effective doses of the single and double IRES-containing rVSV-F vectors will be set up for serial sacrifice (5 animals/time point) at 0, 1, 3, 5, 7, 10 and 14 days post treatment. The abdominal organs will be excised and paraffin-embedded and frozen sections will be obtained. eGFP and RFP expression in the tumors and surrounding normal liver tissues will be examined by fluorescence microscopy; hematoxylin and eosin (H&E) staining will be performed to determine the extent of necrosis within the tumors by morphometric analyses as described (Huang et al., Mol. Ther. 8(3):434-40 (2003) and Shinozaki et al., Mol Ther 9(3):368-376 (2004)) and immunohistochemistry for various immune cell types will be performed to examine the presence of immune cell infiltrates in the lesions. In the remaining liver samples from the same animals, macroscopically visible tumor lesions will be surgically removed, mechanically lysed, centrifuged to remove cellular debris, and the supernatant used to perform plaque assays on BHK-21 cells. Additionally, total RNA will be isolated from an aliquot of the tissue lysates, and viral genomic RNA sequences will be quantified by performing real-time RT-PCR using specific primers. Similar analyses will also be performed on brain and spinal cord tissues of the animals. Kruskal-Wallis one-way ANOVA by ranks will be used to analyze the results obtained from quantitative RT-PCR and plaque assays.

### Example 14

### Antibody-Mediated Depletion of Neutrophils or Natural Killer Cells Enhances Intratumoral rVSV(MΔ51)-LacZ Titer and Enhances Tumor Response

This Example discloses that antibody-mediated depletion of neutrophils or NK cells leads to logarithmic elevations in intratumoral VSV titer and enhanced tumor response in tumor-bearing rats.

Neutrophil and NK cell depletion was accomplished by intravenous administration of rabbit anti-rat polymorphonuclear leukocyte (PMN) antiserum (Wako; Richmond, Va.) and polyclonal rabbit anti-asialo GM1 (Wako Chemical USA, Inc.) 24 hours prior as well as 24 hours post vector infusion. Using a defined dose of 1 mg/200 μl/rat, Buffalo rats harboring multi-focal HCC lesions were randomized to receive either rabbit anti-rat PMN antiserum, anti-asialo GM1 antiserum, or an equal volume of normal rabbit serum (control IgG) in combination with a single hepatic arterial injection of vector. All animals were sacrificed on day 3 after vector administration.

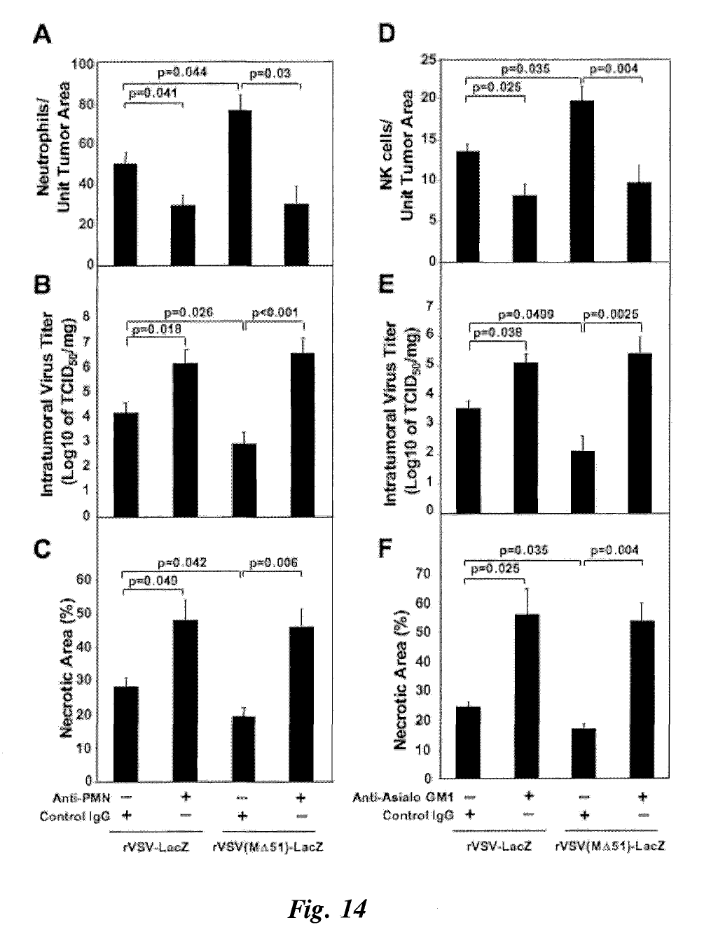

Buffalo rats bearing multi-focal lesions of HCC in the livers were treated with rVSV-LacZ or rVSV(MΔ51)-LacZ at 5×10⁷ pfu/kg, in the presence of rabbit antiserum against rat polymorphonuclear leukocytes (PMNs) to deplete neutrophils, rabbit anti-asialo GM1 to deplete NK cells, or a control rabbit serum. Tumor tissues were obtained from animals sacrificed at day 3 post vector administration, and inflammatory cell were identified by immunohistochemical staining (FIG. 14A).

There were ˜50 neutrophils per unit tumor area in rVSV-LacZ treated animals, which were reduced to ˜30 with anti-neutrophil treatment (p=0.041). Neutrophil numbers were increased up to ˜80 in rVSV(MΔ51)-LacZ treated rats compared to rVSV-LacZ (p=0.044), which were also reduced to ˜30 (p=0.03) with depletion. Lysates of tumor tissues were subjected to TCID₅₀ assays (FIG. 14B), and intratumoral virus titers in rVSV(MΔ51)-LacZ-treated rats were elevated by two-logs with neutrophil depletion (p=0.018). Intratumoral virus titers were decreased by one-log when compared to rVSV-LacZ treated rats (p=0.26), and were restored with neutrophil depletion (p<0.001).

Histology and Immunohistochemistry

Liver samples containing tumor were fixed overnight in 4% paraformaldehyde, then paraffin-embedded. Thin sections were subjected to either H&E staining for histological analysis or immunohistochemical staining using monoclonal antibodies against VSVG protein (Alpha Diagnostic, TX) or myeloperoxidase (MPO) (Abcam, MA). Another set of tumor-containing liver samples was fixed overnight in 4% paraformaldehyde then equilibrated in 20% sucrose in PBS overnight. Frozen sections were subjected to immunohistochemical staining using monoclonal antibodies against NKR-P1A (BD Pharmingen, CA). Semi-quantification of positively stained cells was performed using ImagePro Software (Media Cybernetics, Inc.; Silver Spring, Md.), and immune cell index was calculated as a ratio of positive cell number to unit tumor area (10,000 pixels equals one unit tumor area).

Tumor-containing liver sections were stained histologically and the percentages of necrotic areas within tumors were quantified by morphometric analysis (FIG. 14C). The necrotic areas in tumors from rVSV-LacZ treated animals were increased from 18% to 46% with neutrophil depletion (p=0.049). The necrotic areas were reduced to 18% in tumors from rVSV(MΔ51)-LacZ treated rats (p=0.042) compared to rVSV-LacZ, which were restored to 45% with neutrophil depletion (p=0.006). Similarly, there was a statistically significant reduction in intratumoral contents of NK cells after anti-asialo GM antibody treatment, which was associated with an enhancement of intratumoral virus titers and necrotic areas (FIG. 14D-14F). Compared with the tumors from rats treated with rVSV-LacZ, there were increased intratumoral NK cell accumulation associated with decreased virus titers and necrotic areas in the tumors of rats treated with rVSV(MΔ51)-LacZ (FIG. 14D-14F).

Collectively, these results demonstrate that there was substantial enhancement of neutrophil and NK cell accumulation in tumors treated with rVSV(MΔ51)-LacZ relative to those treated with rVSV-LacZ, which correlated with attenuated replication of rVSV(MΔ51)-LacZ and reduced tumor response in HCC tumors. Moreover, neutrophils and NK cells apparently played a major role in suppressing intratumoral VSV replication, especially after attenuated rVSV(MΔ51) infusion, that could be reversed by their antibody-mediated depletion in vivo, leading to substantially enhanced oncolysis and tumor response.

Assessment of Cytokine Production and Serum Chemistry

Blood samples were collected from the left ventricle 3 days post-virus infusion, at the time of euthanization, and the levels of serum cytokines were determined by ELISA (R&D Systems; Minneapolis, Minn., USA). Serum chemistry including ALT, AST and BUN were performed by the Chemistry Laboratory at Mount Sinai School of Medicine.

Statistical Analyses

For comparison of individual data points, two-sided student t-test was applied to determine statistical significance. Survival curves were plotted according to the Kaplan-Meier method, and statistical significance between the different treatment groups was compared using the log-rank test. Results and graphs were obtained using the GraphPad Prism 3.0 program (GraphPad Software; San Diego, Calif.).

### Example 15

### Construction and In Vitro Characterization of a Recombinant VSV(MΔ51) Vector Expressing the M3 Gene from Murine Gammaherpesvirus-68

This Example discloses the construction and in vitro characterization of a recombinant VSV(MΔ51) vector expressing the M3 gene from murine gammaherpesvirus-68.

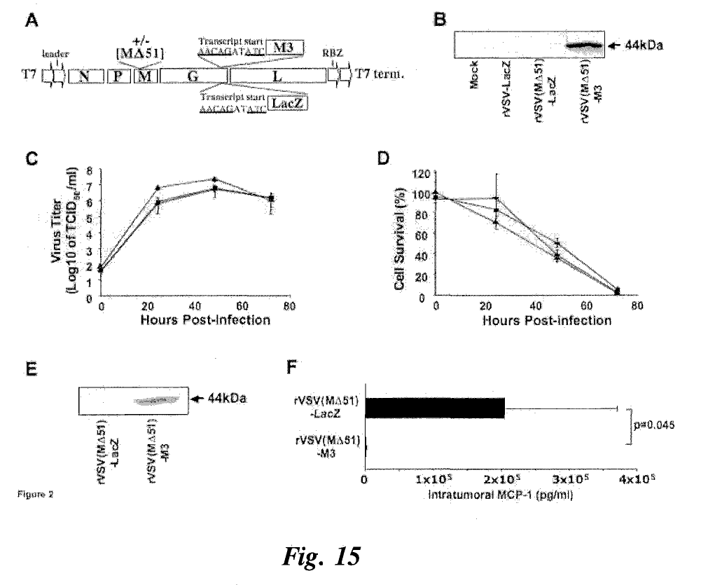

M3 from murine gammaherpesvirus-68 is a broad spectrum chemokine binding protein that suppresses the chemotaxis of inflammatory cells in response to C, CC, CXC and CX3C chemokines with high affinity. Parry et al., J. Exp. Med. 191:573-578 (2000) and van Berkel et al. Journal of Virology 74(15):6741-6747 (2000). The cDNA corresponding to the secreted form of M3 was cloned into the genome of rVSV containing a single methionine deletion at position 51 of the M protein gene (MΔ51) as a new transcription unit (FIG. 15A). Reverse genetics was employed to generate the corresponding recombinant VSV vector, rVSV(MΔ51)-M3, as previously described. Lawson et al., Proc. Natl. Acad. Sci. 92:4477-4481 (1995) and Whelan et al., Proc. Natl. Acad. Sci. 92:8388-8392 (1995).

Rat HCC cells were infected with either rVSV-LacZ, rVSV(MΔ51)-LacZ, or rVSV(MΔ51)-M3 at MOI=10, and controls were mock-infected with culture medium. Conditioned media were collected after five hours and analyzed by Western blotting using a mono-specific anti-M3 antibody.

While there was no detectable M3 protein in the mock, rVSV-LacZ or rVSV(MΔ51)-LacZ infected supernatant, high levels of the protein were present in the supernatant of HCC cells infected with rVSV(MΔ51)-M3 (FIG. 15B). These results indicated that murine gammaherpesvirus M3 was secreted by cells infected with rVSV(MΔ51)-M3.

One concern about constructing recombinant VSV vectors expressing one or more exogenous genes is that this could be detrimental to viral infectivity and titers. To compare the replication kinetics of rVSV(MΔ51)-M3 to that of rVSV(MΔ51)-LacZ and rVSV-LacZ in vitro, TCID₅₀ assays were performed on culture supernatants collected at different time points following infection of the rat HCC cells at an MOI of 0.01 (FIG. 15C). The kinetics of virus replication were similar for all three viruses with no statistically significant differences at all time points, indicating that the new recombinant viruses suffered no significant changes to replication efficiency or overall yield in rat HCC cells in vitro.

To examine the tumor cell killing potential of the new vector, rat HCC cells were infected with rVSV(MΔ51)-M3, rVSV(MΔ51)-LacZ or rVSV-LacZ at an MOI of 0.01. The cytopathic effects on the cells were quantified by MIT assays and expressed as a percentage of mock-infected cells at each time point. The kinetic profiles of cell killing caused by alt three viruses were very similar and without statistical significant differences at all time points, with nearly all of the cells being killed within 72 hours post-infection (FIG. 15D). These results demonstrate that rVSV(MΔ51)-LacZ and rVSV(MΔ51)-M3 were able to kill rat hepatoma cells as effectively as rVSV-LacZ in vitro.

To determine the secreted M3 expression as well as the chemokine level in tumors after viruses infection in vivo, multi-focal HCC tumor-bearing rats were infused with rVSV(MΔ51)-LacZ or rVSV(MΔ51)-M3 at 5.0×10⁹ pfu/gg via the hepatic artery. Three days after virus injection, tumors were harvested and homogenized for detection of M3 by Western blotting and for measurement of MCP-1 by ELISA. High levels of secreted M3 protein was present in the tumors infused with rVSV(MΔ51)-M3 but not in those infused with rVSV(MΔ51)-LacZ (FIG. 15E). The intratumoral chemokine MCP-1 protein level was significantly lower in rats administrated with rVSV(MΔ51)-M3 than that with rVSV(MΔ51)-LacZ (FIG. 15F, p=0.045).

These results indicated that elevated intratumoral M3 expression after rVSV(MΔ51)-M3 infusion was associated with reduced intratumoral levels of a chemokine in vivo.

### Example 16

### Suppression of Neutrophil and NK Cell Accumulation in the HCC Lesions of rVSV(MΔ51)-M3 Treated Rats

This Example demonstrates that suppression of neutrophil and NK cell accumulation in the HCC lesions of rVSV(MΔ51)-M3 treated rats.

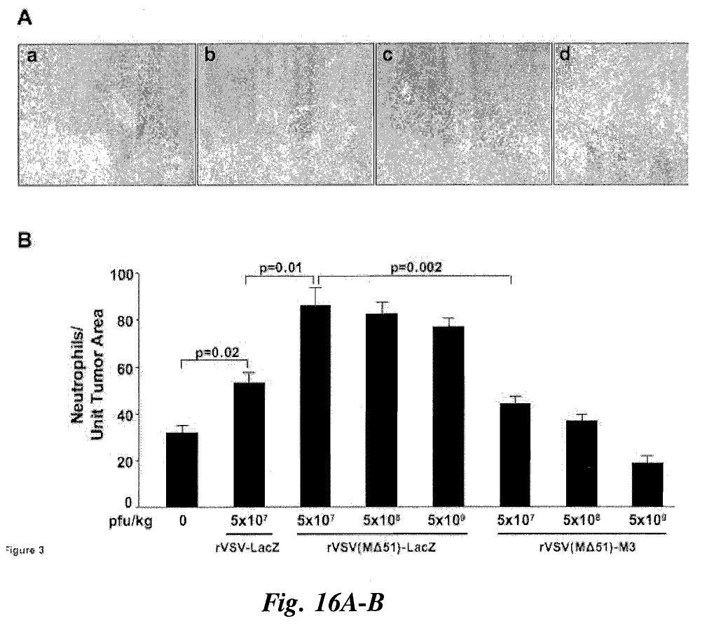

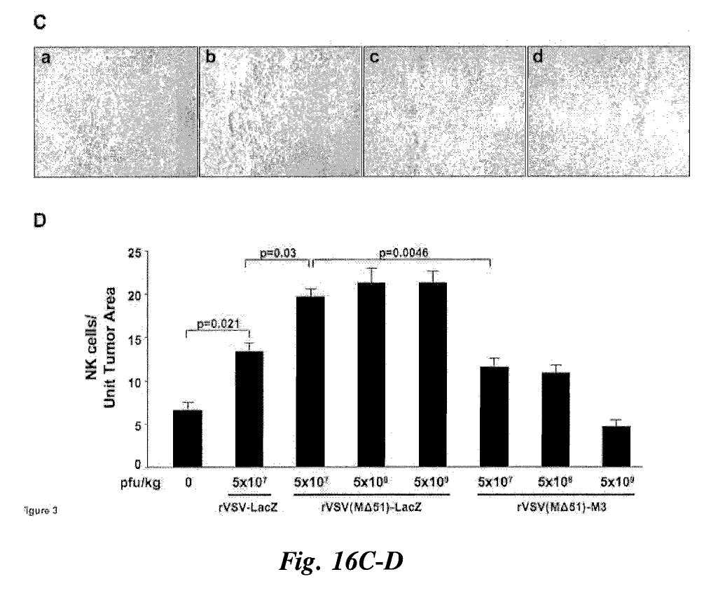

To evaluate whether secretion of the M3 protein by tumor cells infected with rVSV(MΔ51)-M3 could inhibit inflammatory cell accumulation in vivo, rats bearing multi-focal HCC lesions ranging from 1-10 mm in diameter were treated with either buffer, rVSV-LacZ at its MTD (5.0×10⁷ pfu/kg), or rVSV(MΔ51)-LacZ or rVSV(MΔ151)-M3 at the equivalent or higher doses (5.0×10⁷, 5.0×10⁸ and 5.0×10⁹ pfu/kg) via hepatic artery infusion. On day 3 after treatment, animals were sacrificed and tumor-containing liver sections were stained for neutrophils using anti-MPO (FIG. 16Aa) and NK cells using anti-NKR-P1A (FIG. 16Ac).

Semi-quantification of marker-positive cells using ImagePro software revealed that there was a substantial accumulation of neutrophils and NK cells in the lesions of rVSV(MΔ51)-LacZ vs. rVSV-LacZ treated rats (FIG. 16Ab, p=0.01 and FIG. 16Ad, p=0.03, respectively), which were substantially reduced after rVSV(MΔ51)-M3 treatment at the same dose (FIG. 16Ac, p=0.002 and FIG. 16Ad, p=0.0046, respectively). Additionally, there appeared to be dose-dependent suppression of intratumoral neutrophil and NK cell accumulation in rVSV(MΔ51)-M3 treated rats (FIGS. 16Cb and 16Cd). Taken together, these results indicate that the chemotaxis of neutrophils and NK cells to the tumor site was enhanced by VSV(MΔ51) but substantially inhibited by vector-mediated expression of M3.

### Example 17

### Logarithmic Elevation of Intratumoral rVSV(MΔ51)-M3 Titer and Enhanced Tumor Response in Tumor-Bearing Rats

This Example demonstrates the logarithmic elevation of intratumoral rVSV(MΔ51)-M3 titer and enhanced tumor response in tumor-bearing rats.

To assess the in vivo effect of combining the M protein deletion mutant with vector-mediated intratumoral M3 expression on intratumoral virus replication and oncolysis, tumor-bearing rats were treated with either buffer, rVSV-LacZ at its MTD (5.0×10⁷ pfu/kg), or rVSV(MΔ51)-LacZ or rVSV(MΔ51)-M3 at the equivalent or higher doses (5.0×10⁷, 5.0×10⁸, and 5.0×10⁹ pfu/kg) via hepatic artery infusion. Animals were sacrificed on day 3 after treatment and tumor samples were collected and fixed for histological and immunohistochemical staining, as well as snap-frozen for intratumoral viral titer quantification by TCID₅₀ analysis.

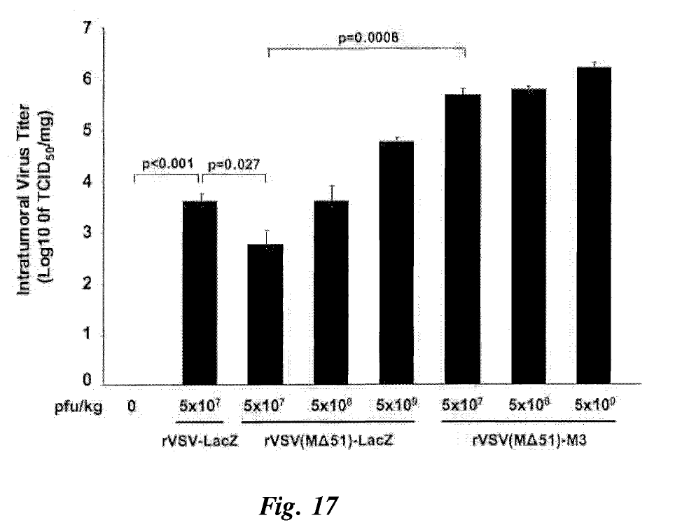

While rVSV-LacZ infusion resulted in virus titers of less than 10⁴ TCID₅₀/mg of tumor tissue, an equivalent dose of rVSV(MΔ51)-LacZ led to a one-log attenuation in intratumoral virus titer (FIG. 17, p=0.027). The same dose of rVSV(MΔ51)-M3 resulted in a three-log enhancement in intratumoral virus titer as compared to rVSV(MΔ51)-LacZ (FIG. 17, p=0.0008).

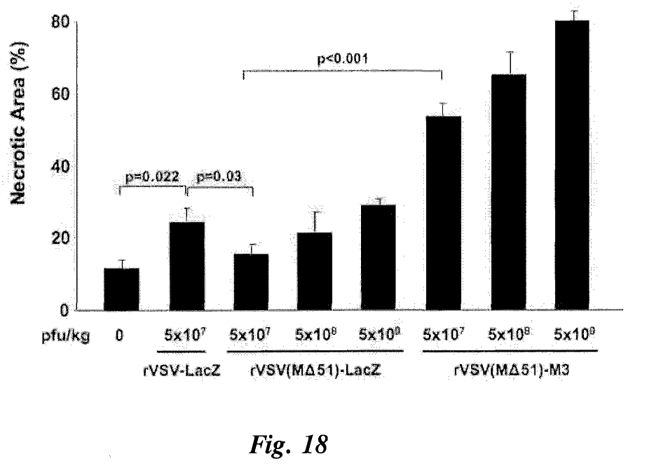

To examine the impact of enhanced intratumoral virus replication on tumor response, tumor-containing liver sections from the animals in the above experiment were examined by H&E staining, and the necrotic areas were quantified by morphometric analysis. The extents of tumor necrosis were reduced in the rVSV(MΔ51)-LacZ treatment group compared to the rVSV-LacZ control vector group (FIG. 18, 15% vs. 23%, p=0.03), and a significant enhancement of tumor response was observed in rats treated with rVSV(MΔ51)-M3 vs. those treated with an equivalent dose of the rVSV(MΔ51)-LacZ vector (FIG. 18, 50% vs 15%, p<<0.001). There also appeared to be a dose dependence in tumor response to rVSV(MΔ51)-M3 administration, which was further elevated to 80% at the highest dose.

### Example 18

### Substantial Survival Prolongation in Multi-Focal HCC-Bearing Rats Treated with rVSV(MΔ51)-M3

This Example demonstrates survival prolongation in multi-focal HCC-bearing rats treated with rVSV(MΔ51)-M3 as compared to VSV(MΔ51).

In order to determine whether the attenuated oncolytic potency of rVSV(MΔ51)-LacZ can be overcome by vector-mediated expression of the M3 gene, rats bearing multi-focal lesions of HCC were treated with either buffer, rVSV-LacZ at its MTD, and rVSV(MΔ51)-LacZ or rVSV(MΔ51)-M3 at equivalent or higher doses, via hepatic artery infusion. The animals were monitored daily for survival.

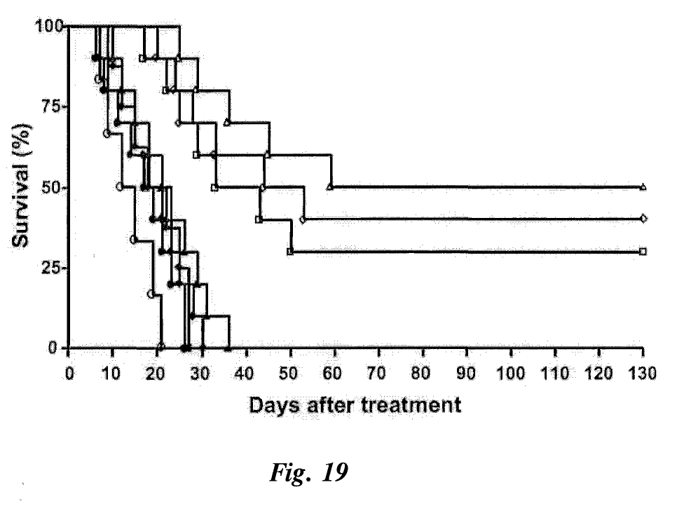

rVSV-LacZ treatment prolonged median animal survival from 14 to 17 days (FIG. 19, p=0.048 vs. buffer). Following treatment with rVSV(MΔ51)-LacZ at doses of 5.0×10⁷, 5×10⁸, and 5.0×10⁹ pfu/kg, median survival was 21, 22, and 23 days, respectively. All animals expired by day 35, and there were no statistical significant differences between various dose level cohorts and from rVSV-LacZ treated animals (FIG. 19).

rVSV(MΔ51)-M3 treatment resulted in highly significant prolongation of median survival from 21 days to 33 days when compared to rVSV(MΔ51)-LacZ treated animals at 5.0×10⁷ pfu/kg (p=0.004), with 3 of H) animals (30%) achieving long-term survival. The median survival advantage was further increased to 44 and 59 days in HCC-bearing rats given 5.0×10⁸ and 5.0×10⁹ pfu/kg of rVSV(MΔ51)-M3, with concomitant increases in long term survival to 40% and 50%, respectively.

The surviving rats in the rVSV(MΔ51)-M3 treatment groups were sacrificed on day 130 and evaluated for residual malignancy. There were no visible tumors within the liver or elsewhere, and there was no histological evidence of residual tumor in all of the major organs. These results indicate that the attenuated oncolytic potency of VSV(MΔ51) can be completely overcome by vector-mediated expression of the M3 gene. Importantly, the results indicate that multi-focal lesions of up to 10 mm in diameter at the time of treatment had undergone complete remission in a significant fraction of the animals treated with rVSV(MΔ51)-M3, which translated into long-term, tumor-free survival.

### Example 19

### Absence of Systemic and Organ Toxicities Following rVSV(MΔ51)-M3 Treatment in Tumor-Bearing Rats

This Example demonstrates the absence of systemic and organ toxicities following hepatic artery infusion of rVSV(MΔ51)-M3 in tumor-bearing rats.

Safety is of the utmost concern when utilizing genetic strategies to enhance oncolytic virus potency, considering that they are capable of evading the host anti-viral inflammatory responses. Consistent with previous reports using VSV(MΔ51)-based vectors, all rVSV(MΔ51)-LacZ and rVSV(MΔ51)-M3 treated animals showed no significant weight loss, dehydration, piloerection, limb paralysis or lethality even at doses as high as 5.0×10⁹ pfu/kg, which is 2-logs higher than the MTD of wild-type VSV.

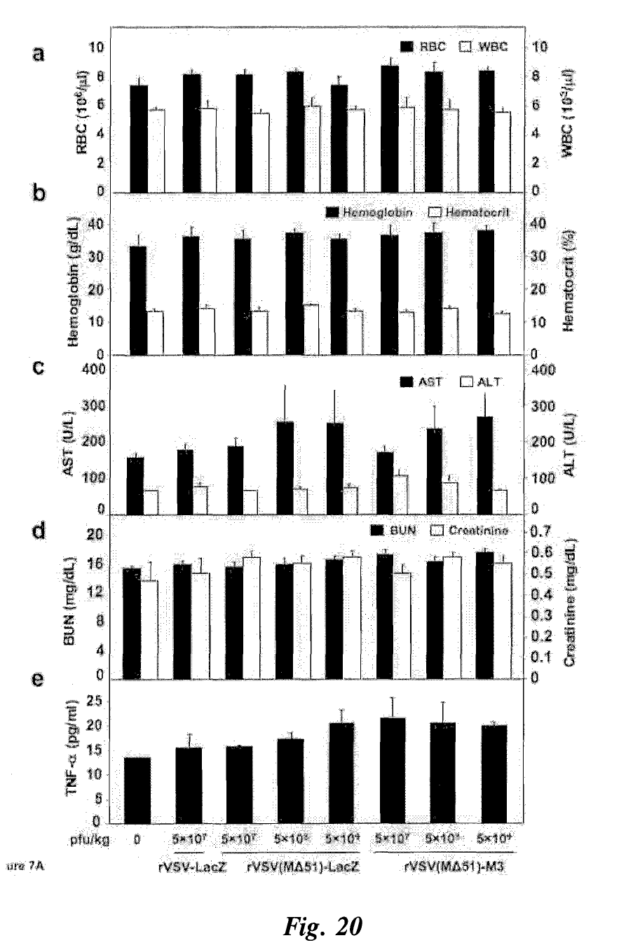

To assess potential systemic and organ toxicities, CBC, serum ALT, AST, BUN, creatinine and serum proinflammatory cytokine levels were measured at day 3 after hepatic artery infusion of buffer, rVSV-LacZ at its MTD, and rVSV(MΔ51)-LacZ or rVSV(MΔ51)-M3 at equivalent or higher doses. There were no abnormal changes in red blood cells (RBC), white blood cells (WBC), hemoglobin and hematocrit following treatment with any of the viruses at all doses used (FIGS. 20Aa and 20Ab), indicating normal hematologic functions. Both AST and ALT were elevated somewhat in the buffer and all vector treated groups due to the presence of HCC lesions, and there were no significant differences between any of the treatment groups indicating that none of these three viruses have any additional toxic effect on liver function (FIG. 20Ac). There were also no increases in BUN or creatinine levels, demonstrating that there was no nephrotoxicity (FIG. 20Ad). The serum concentrations of the proinflammatory cytokine, INF-4 were comparable between the buffer and all rVSV vector treatment groups, and were >2-logs below the concentrations associated with systemic toxicity in animals and in human clinical trials (the toxic threshold of TNF-α in clinical trails is 3000 pg/ml). Gaddy and Lyles, J. Virol. 79:4170-4179 (2005); FIG. 20Ae. The serum concentration of another proinflammatory cytokine, IFN-γ was undetectable in all groups (<31.2 pg/ml), indicating that there was no systemic proinflammatory cytokine response in the immune-competent rats.

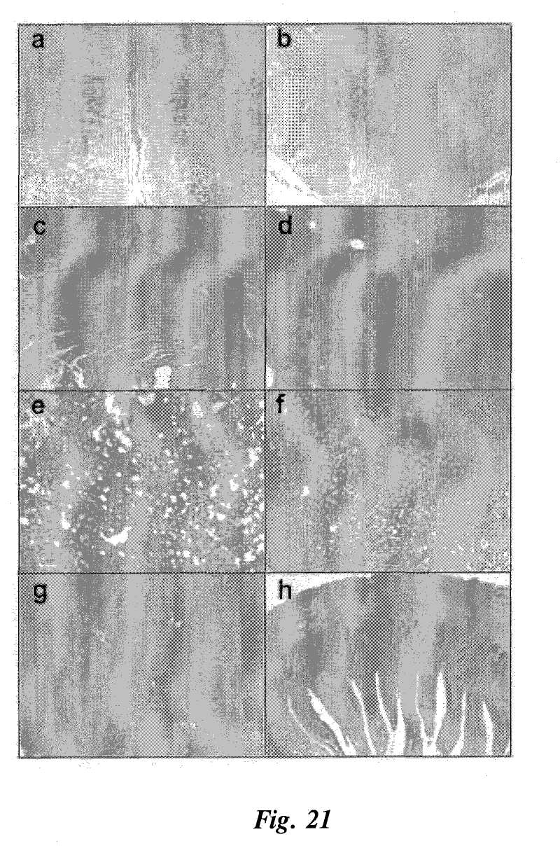

Histological sections of the liver and other major organs including the brain, spinal cord, lung, heart, kidney, spleen, duodenum were examined at 3 days after virus infusion and these tissues were completely normal with no inflammatory cell infiltration (FIG. 21), indicating that there was no organ toxicity in animals injected with rVSV(MΔ51)-M3.

### Example 20

### Evaluation of Safety and Efficacy of rVSV(MΔ51)-M3 in Humans

This Example discloses human experiments to demonstrate the safety and efficacy of rVSV(MΔ51)-M3 in patients with unresectable malignant neoplasms in the liver.

The toxicity of rVSV(MΔ51)-M3 may be studied by administering escalating doses of the recombinant VSV by hepatic arterial injections via a percutaneously placed hepatic arterial catheter into patients with primary or metastatic non-hematologic neoplasms in the liver. rVSV(MΔ51)-M3 doses may be escalated in 7 dose level cohorts of three patients each.

The starting dose of rVSV(MΔ51)-M3 is 5.0×10⁶ pfu/kg (2.5×10⁸ pfu/patient), which is three logs below the MTD from the rat studies. Three evaluable subjects are entered to each dose level cohort. rVSV(MΔ51)-M3 doses are escalated in half-log increments up to 5.0×10⁹ pfu/kg (2.5×10¹¹ pfu/patient). Subjects are considered to be evaluable if they received the planned virus injection and are able to be followed for at least four weeks.

Dose limiting toxicity (DLT) is defined as any grade >3 toxicity, including hematologic toxicities, but not constitutional symptoms (fever, fatigue). If DLT is observed in none of three patients at a cohort level, rVSV(MΔ51)-M3 dose is escalated to the next cohort level. If DLT is observed in two out of three patients at a cohort level, further enrollment at that dose level will cease and no further dose escalation is performed. If DLT is observed in one out of three patients at a cohort level, then three additional patients will be treated at the same level. If DLT is seen in one of the additional patients, then further enrollment at that dose level will cease, three additional patients will be added to the previous cohort (now defined as the MTD), no further dose escalation will be performed, and the FDA will be notified. If DLT is not seen in the additional three patients, the MTD is not reached and dose escalation to the next cohort level will continue. If DLT is not seen at the highest planned cohort level (#8), the protocol will be amended at that time to include further dose escalations, and the trial will not proceed until all regulatory approvals are obtained. The maximal tolerated dose (MTD) for rVSV(MΔ51)-M3 is defined as the highest cohort level at which less than two instances of DLT are observed among six patients treated. Dose escalation to the next cohort level is performed only after the last patient on the current level has completed treatment, and all toxicities up to 4 weeks following rVSV(MΔ51)-M3 injection have been reviewed.

21 to 33 patients are treated in this trial, depending on toxicities encountered. The anticipated age range will be 18 to 85, since HCC and CRC are rare in subjects under the age of 18. On the day of virus injection, each study subject is also administered piperacillin/tazobactam 3.375 gm IV (or levofloxacin 500 mg IV for subjects with a history of penicillin allergy). Percutaneous hepatic arterial catheterization and hepatic angiography are performed, followed by assessment of hepatic angiography and decision to administer rVSV(MΔ51)-M3.

Hepatic Arterial Catheterization and Angiography Procedure

The study subject is placed in the supine position on the fluoroscopic table. EKG, blood pressure and pulse oximetry are monitored continuously during the procedure. The groin area is prepped and draped in a standard sterile manner with iodine.

The area over the common femoral artery is localized by palpation and fluoroscopy. 1% lidocaine is infiltrated into the skin and subcutaneous tissues over this vessel. The vessel is entered with a 10 gauge thin wall needles. A 0.035 Benton guidewire is advanced through the needle into the abdominal aorta. The needle is removed over the wire and a 5 French vascular sheath (Terumo, Tokyo) is advanced into the femoral artery. The sidearm of the sheath is placed to a continuous saline flush.

A 5 French Sos 1 selective catheter (Angiodynamics, Queensbury, N.Y.) or a 5 French Mickelson catheter (Cook, Bloomington, Ind.) is advanced into the celiac and superior mesenteric arteries. Injections of 20 ml of iopamidol 61% (Isovue—300 Bracco) at 4 ml/sec are used to opacify these two vessels and their branches. Images are recorded at 3 frames/sec for three seconds and one frame per second until the venous phase is identified on the monitor. The angiographic images are correlated with the prior CT/MRI images so that the proper vessels are selected for subselective catheterization.

The angiographic images are correlated with the prior CT/MRI images, and evaluated for tumor hypervascularity, absence of hepatofugal portal flow, portal venous thrombosis and for arterioportal/arteriovenous shunting.

Depending on the extent and location of the tumor either the proper hepatic, right or left hepatic arteries are selectively entered with a renegade Hi-Flo catheter (Boston Scientific, Natick, Mass.). The micro-catheter passes through the lumen of the 5 French Sos selective or Mickelson catheters. Using a pre-curved 0.018 wire, the appropriate branch is entered and the microcatheter advanced to the desired site. Correct position is confirmed by fluoroscopy and by recording images after a 1-2 ml injection of contrast material. The microcatheter will then be flushed with saline.

rVSV(MΔ51)-M3 Hepatic Arterial Injection

A micro-catheter is in place in the hepatic vessel to be used for virus injection. An aliquot of rVSV(MΔ51)-M3 is thawed, and the desired volume containing the assigned virus is diluted with sterile normal saline to a total volume of 25 ml for injection in the study subject.

The micro-catheter is flushed with saline and the rVSV(MΔ51)-M3 is injected by manual push over live to ten minutes. Following injection of the rVSV(MΔ51)-M3, a final image is obtained to confirm that the micro-catheter does not moved during delivery of the rVSV(MΔ51)-M3 virus.

The microcatheter and sheath are removed and the percutaneous catheter injection site is pressed manually for at least 15 minutes to ensure no bleeding from the catheter site. The subject will remain on bed rest until six hours after removal of the catheter. The study subject will have blood samples collected for study monitoring and results reviewed.

The rVSV(MΔ51)-M3 vector used in this human study is derived from the VSV-Indiana subtype. Transmission is primarily via close contact (transcutaneous or transmucosal) or from parenteral exposure via sandflies. The incubation period is generally less than 24 hours. To address the issue of rVSV(MΔ51)-M3 transmission, patient samples are assessed for dissemination via blood, secretions and vesicles. Throat Nasal swabs, stool, urine and blood samples are collected from study subjects at baseline prior to study procedures, and at one and one six days after each the rVSV injection, and tested for the presence of VSV.

In addition, if cutaneous or oropharyngeal vesicles develop in trial subjects, the vesicle is swabbed and tested for the presence of VSV. The presence of infectious VSV is assessed by in vitro plaque assays. Patients are released only after the levels of VSV in the blood, urine and nasal swabs fall below the level of detection for the plaque assay.

Purified rVSV(MΔ51)-M3 is suspended in formulation buffer. (10 mM. Tris, pH 7.5/150 mM NaCl/10 mM EDTA) and aliquotted at suitable titers into cryovials. The filled vials are stored at or below ⁻60° C. rVSV(MΔ51)-M3 is injected via hepatic arterial catheterization into the liver as previously described.

Toxicity is assessed from grades 0 to 4 according to common toxicity criteria (version 3.0) from the National Cancer Institute. Tumor response and progression is assessed by the RECIST criteria. All measurable lesions up to a maximum of 5 lesions are identified as target lesions. The longest diameter of these lesions is measured and recorded at baseline. The sum of the longest diameters of the target lesions is calculated and used as a reference for determination of overall tumor regression and response (sum-LD). Other lesions identified as non-target lesions are identified and recorded at baseline. Measurability is arbitrarily defined as reproducibility of simultaneous measurements, within 50% by independent observers.

Tumor response (target lesions) is categorized as follows: (1) Complete=complete disappearance of all target lesions on two assessments four weeks apart; (2) Partial=>30% decrease in the sum-LD of target lesions on two assessments four weeks apart; (3) Stable=<50% decrease or <20% increase in the sum-LD of target lesions; (4) Progression=>20% increase in the sum-LD of target lesions.

Tumor response (non-target lesions) is categorized as follows: (1) Complete=Complete disappearance of all non-target lesions AND normalization of serum AFP; (2) Progression=Appearance of one or more new lesions OR unequivocal progression of non-target lesions; (3) Non-complete response (non-CR)/non-progression (non-PD)=Persistence of any non-target lesion OR persistent elevation of serum AFP above upper limit or normal. The best overall response is assessed from the start of treatment until disease progression incorporating target and non-target lesions.

One objective of this human clinical trial are to assess the safety and to determine the maximal tolerated dose (MTD) of rVSV(MΔ51)-M3. The definitions of dose limiting toxicity (DLT) and MTD have been described. Toxicity results are presented for each patient and summarized by dose level using descriptive statistics. All toxicities are individually listed and summarized within each dose level cohort by calculating the number (and proportion) of patients experiencing severe (grade >3) toxicity and the number of patients experiencing moderate severe (grade >2) toxicity. In addition, for each dose level cohort, the median toxicity grade (and range) for each toxicity endpoint is calculated. Hepatic toxicity laboratory parameters such as serum total bilirubin, ALT and AST have the median and range for peak levels computed for each dose level cohort.

Serum neutralizing antibody titers to VSV are measured pre-treatment and on various days post-treatment (days 2, 3, 6, 15, and 29). Treatment effect for each patient is measured as paired differences between pre and post measurements of these immune parameters at various times. Transformation of the data is performed by, e.g., log transformation, and hence treatment effect is expressed on a log scale. By evaluating six patients at the MID, a power of 80% for detecting a mean treatment effect of 1.5 standard deviations (standard deviation of differences) can be determined for a two-sided test at the 0.05 level of significance.

In addition to serum neutralizing antibodies to VSV, tumor markers (AFP) are also measured pre-treatment and on various days post-treatment (days 15, 30, 44, and 58). Treatment effect for each patient on this parameter is calculated in the same way as for antibodies to VSV.

Elevations of serum IL12, IFNγ, IL6, and TNFα levels are monitored. Blood is obtained three times prior to treatment, and then on days 2, 3, 4, 5, 6, 8, 11, and 15. The cytokine assays is measured in duplicate by ELISA. For each patient, the mean of the three pre-treatment values is used as the baseline value. Data from each type of serum cytokine is graphed over time for each individual patient. Peak levels are obtained from each patient and are summarized for each dose level cohort by calculating the median peak serum cytokine level (and range of peak levels). The day the peak level occurs and the time for levels to return to baseline levels are also noted. To test for a dose effect on serum cytokine peak levels, linear regression analysis is conducted, using the log 10 transformation on dose levels. Assumptions underlying the method of linear regression are assessed for the data before proceeding with analyses. Transformation of the cytokine peak level data is performed if appropriate.

Results obtained pre-treatment and each time point post-treatment are compared by the paired t-test for each cohort level separately. Transformation of the data is performed if appropriate. Also, serum neutralizing antibody titer to VSV data over time is plotted separately for each patient and summarized by cohort level. Data is checked for violations of the basic assumptions underlying these procedures before their application. In addition, tumor response data is summarized by calculating the percentage of patients achieving a CR or PR.

Once the maximal tolerated dose (MTD) of rVSV(MΔ51)-M3 in humans has been determined by the Phase I clinical trial described above, Phase II and Phase III clinical trials using a safe dose of rVSV(MΔ51)-M3 will be launched in succession to determine its efficacy in Hepatocellular Carcinoma and other cancers.

### Example 21

### Additional Inflammation Suppressive Genes that can Enhance the Potency of Oncolytic Viruses

This Example discloses additional inflammation suppressive genes that enhance the potency of the oncolytic viruses disclosed herein.

Additional CKBP Genes to Enhance the Anti-tumor Effects of Oncolytic Viruses Certain orthopoxviruses, such as vaccinia virus and myxoma virus, express members of the T1/35 kDa family of secreted proteins which bind with members of the CC and CXC superfamilies of chemokines, and effectively block leukocyte migration in vivo (Graham et al., Virology 229:12-24 (1997)). More recently, it was demonstrated that ectromelia virus (EV) expresses a soluble, secreted 35 kDa viral chemokine binding protein (EV35; SEQ ID NOs: 7 and 8) with properties similar to those of homologous proteins from the T1/35 kDa family. It was demonstrated in vitro that EV35 specifically and effectively sequesters and binds CC chemokines (Smith'et al., Virology 236:316-327 (1997) and Baggiolini, Nature 392:565-568 (1998)).

The inflammatory response to virus challenge is characterized by the migration and activation of leukocytes, which initiate the earliest phases of antiviral immune activation. Zinkernagel, Science 271:173-178 (1996). The larger DNA viruses encode immunomodulatory proteins, which interact with a wide spectrum of immune effector molecules, as a method of evading this response. McFadden and Graham, Semin. Virol. 5:421-429 (1994). In particular, certain orthopoxviruses, such as vaccinia virus and myxoma virus, express members of the T1/35 kDa family of secreted proteins which bind with members of the CC and CXC superfamilies of chemokines; and effectively block leukocyte migration in VIVO. Graham et al., Virology 229:12-24 (1997). More recently, it was demonstrated that ectromelia virus (EV) expresses a soluble, secreted 35 kDa viral chemokine binding protein (EV35) with properties similar to those of homologous proteins from the T1/35 kDa family. It was demonstrated in vitro that EV35 specifically and effectively sequesters and binds CC chemokines, and it is speculated that in vivo chemokine binding activity would inhibit migration of monocytes, basophils, eosinophils, and lymphocytes. Smith et al., Virology 236:316-327 (1997); Baggiolini “The Chemokines” 1-11 (ed. I. Lindley, Plenum, NY (1993)); and Baggiolini, Nature 392:565-568 (1998).

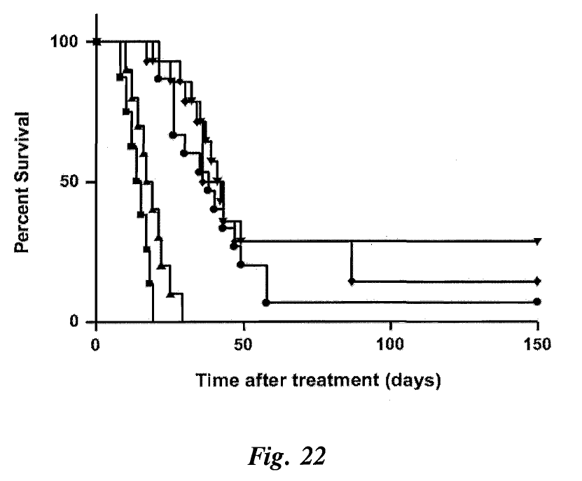

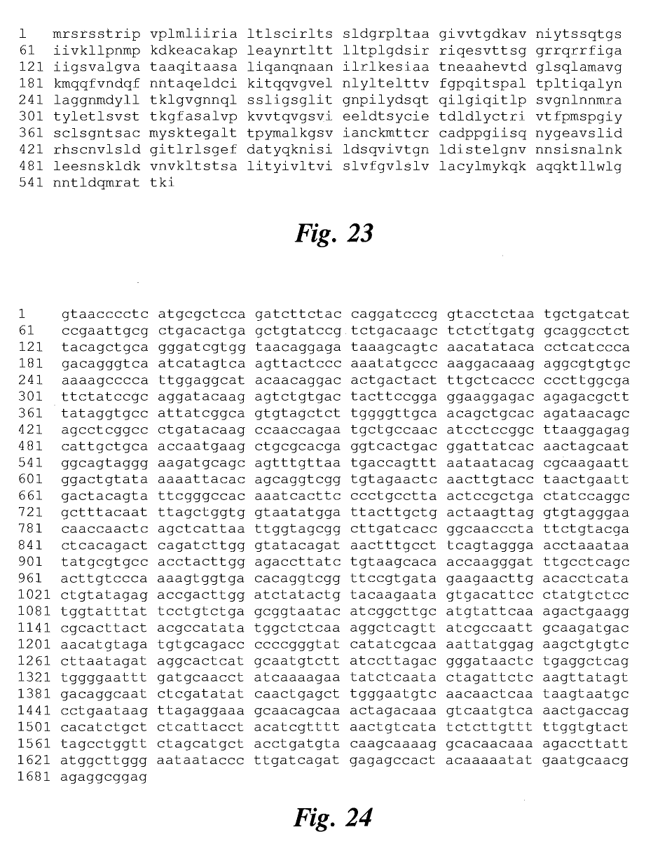

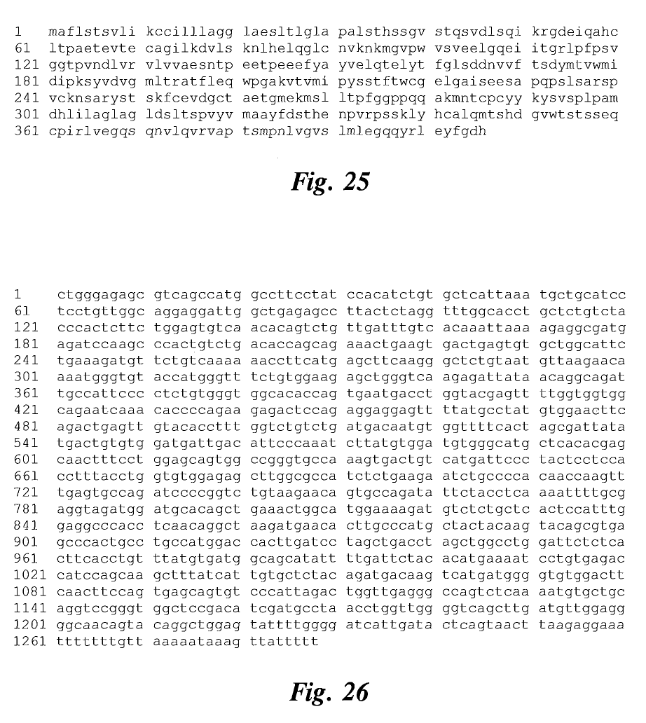

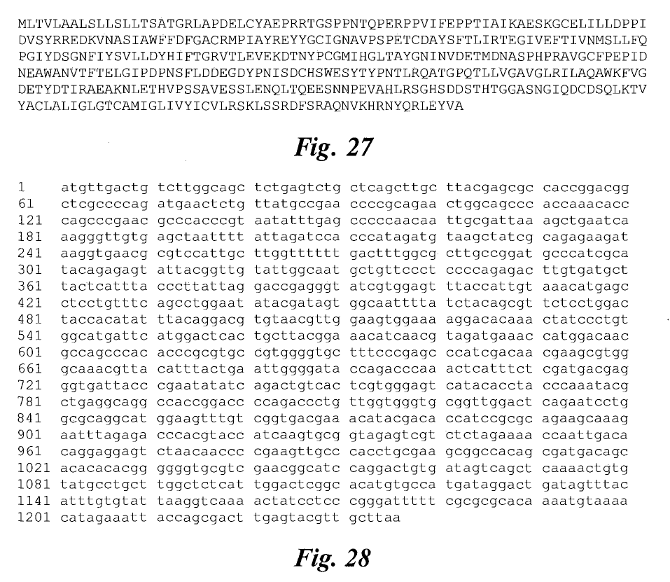

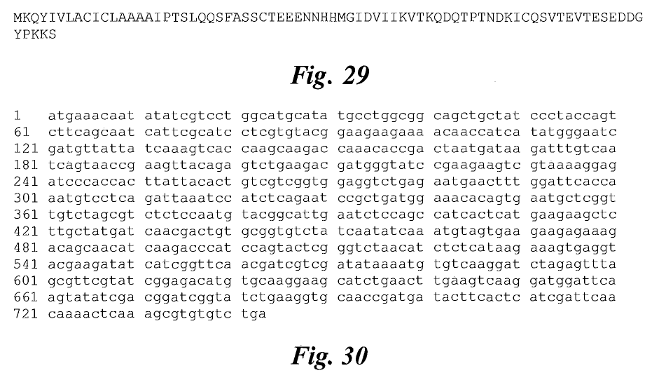

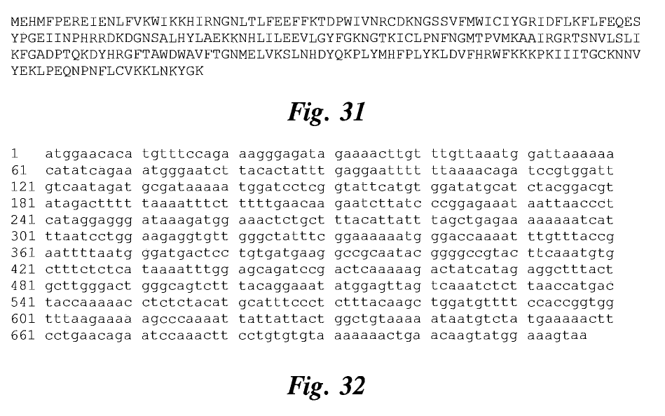

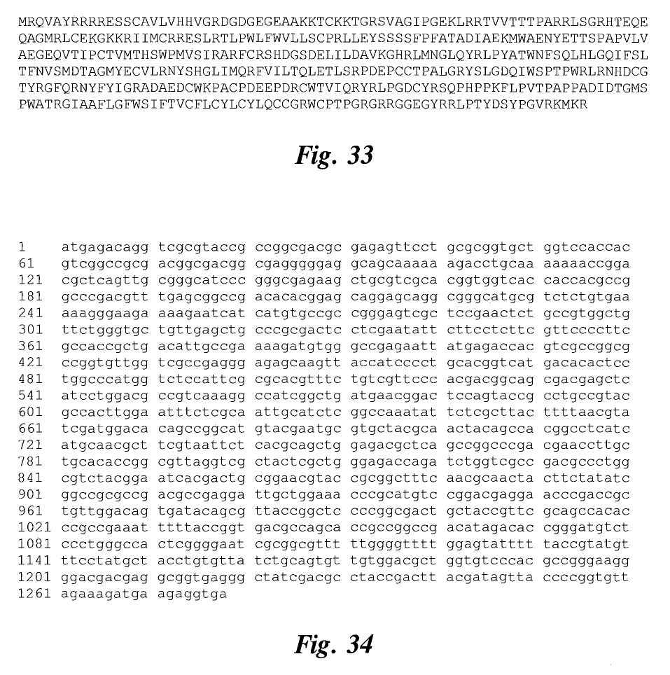

The EV35 gene was obtained by PCR amplification and inserted into the full-length pVSV-XN2 plasmid, as an additional transcription unit in between endogenous G and L proteins. The recombinant rVSV-EV35 virus was rescued using the established method of reverse genetics (Lawson et al., Proc. Natl. Acad. Sci. 92:4477-4481 (1995) and Whelan et al., Proc. Natl. Acad. Sci. 92:8388-8392 (1995)) and shown to produce substantive prolongation of survival over PBS and rVSV-F in rats bearing multi-focal lesions of HCC in the liver after hepatic artery infusion (FIG. 22). These results further demonstrate that the anti-tumor efficacy of oncolytic viruses can be substantively enhanced by vector-mediated vCKBP expression.

NK-Suppressive Genes to Enhance the Anti-Tumor Effects of Oncolytic Viruses

The UL141 gene from the human cytomegalovirus (UL141_{HCMV}) is a powerful inhibitor of NK cell function. Braud et al., Curr Top Microbiol Immunol. 269:117-129 (2002) and Tomasec et al., Nature Immunology 6:181-188 (2005). UL141_{HCMV} mediates the evasion of NK killing of virus-infected cells by blocking the surface expression of CD155, which is a ligand for NK cell-activating receptors CD226 and CD96. Bottino, J Exp Med 198:557-567 (2003) and Fuchs et al., J. Immunol 172:3994-3998 (2004).

The UL141_{HCMV} gene was obtained by PCR amplification and inserted into the full-length pVSV-XN2 plasmid, as an additional transcription unit between endogenous G and L proteins. A genetically modified rVSV vector expressing UL141_{HCMV} was rescued by reverse genetics (Lawson et al., Proc. Natl. Acad. Sci. 92:4477-4481 (1995) and Whelan et al., Proc. Natl. Acad. Sci. 92:8388-8392 (1995)) and tested in rats bearing multi-focal lesions of HCC in the liver. Substantial prolongation of survival in the treated animals was achieved (FIG. 22).

Additional viral genes are associated with NK cell inhibition, such as M155 from murine CMV (Lodoen et al., J. Exp. Med. 200:1075-1081 (2004)) and the K5 gene from Kaposi's Sarcoma-associated Herpesvirus (Orange et al., Nature Immunology 3:1006-1012 (2002)), among others. The results presented herein indicate that each of these NK suppressive genes can be inserted into the genomes of oncolytic viruses to substantially enhance their anti-tumor efficacy.

NF-κB-Suppressive Genes to Enhance the Anti-tumor Effects of Oncolytic Viruses

The NF-κB family of transcription factors regulates expression of numerous cellular genes, and its activation plays a major role in the protective response of cells to viral pathogens by launching an inflammatory response, and modulating the immune reaction. Santoro et al., EMBO J. 22:2552-2560 (2003). Therefore, the ability of a virus to regulate and evade NF-κB activation is critical for viral propagation. To this end, several viruses encode proteins which have recently been demonstrated to specifically interfere with NF-κB function. Bowie et al., Proc. Natl. Acad. Sci. U.S.A. 97:10162-10167 (2000); Akari et al, J. Exp. Med. 194:1299-1311 (2001); Bour et al., J. Biol. Chem. 276:15920-15928 (2001); Revilla et al., J. Biol. Chem. 273:5405-5411 (1998); and Zheng et al., J. Virol. 81:11917-11924 (2007).

One of the best characterized viral proteins with NF-κB inhibitory function is the A238L protein encoded by African Swine Fever Virus (ASFV; SEQ ID NOs: 9 and 10). There are several mechanisms by which A238L may act to inhibit NF-κB activation. In non-stimulated cells, NF-κB remains in the cytoplasm in an inactive state, bound to the inhibitor of NF-κB (IκB). Upon activation by a variety of stimuli, including viral infection, IκB becomes phosphorylated by IκB kinase (IKK), followed by ubiquitination and finally, degradation by the proteasome, which allows NF-κB to be transported to the nucleus, where it can regulate transcription of downstream genes. Karin, J. Biol. Chem. 274:27339-27342 (1999). Due to sequence homology between ankyrin repeats in A238L and IκB, it was demonstrated that A238L binds directly to NF-κB. Furthermore, because A238L does not contain the serine residues that are phosphorylated by IKK, A238L is not degraded following stimulation of the NF-κB pathway. Tait et al., J. Biol. Chem. 275:34656-34664 (2000) and Dixon et al., Vet Immunol Immunopathol. 100:117-134 (2004). In this aspect, A238L acts as a dominant negative inhibitor of NF-κB by retaining the protein in the cytoplasm.

A second mechanism by which A238L exerts its activity involves the fact that this protein also resides in the nucleus. Here it inhibits NF-κB activation by preventing its binding to target DNA sequences, and can also displace pre-formed NF-κB transcription complexes from DNA. Revilla et al., J. Biol. Chem. 273:5405-5411 (1998) and Silk et al., J. of Gen. Virol. 88:411-419 (2007).

Additionally, A238L has been shown to interfere with several other host factors, such as calcineurin phosphatase, TNF-α, and COX-2. Dixon et al., Vet Immunol Immunopathol. 100:117-134 (2004); Powell et al., J. Virol 70:8527-8533 (1996); Granja et al., J. Virol. 80:10487-10496 (2006); and Granja et al., J. Immonol. 176:451-462 (2006). The A238L protein thus has the potential to act as a potent immunosuppressant by inhibiting transcriptional activation of several key immune response genes.

A recombinant VSV vector was constructed such that the A238L gene was expressed as an additional transcription unit inserted between the endogenous VSVG and VSVL genes. The A238L gene (SEQ ID NO: 10) was synthesized (GenScript; Piscataway, N.J.) with Xho I and Nhe I restriction sites for insertion into the pVSV-XN2 vector. The resulting plasmid was then used to rescue the corresponding rVSV vector by reverse genetics technique. Lawson et al., Proc. Natl. Acad. Sci. U.S.A. 92:4477-4481 (1995) and Whelan et al., Proc. Natl. Acad. Sci. U.S.A. 92:8388-8392 (1995).

Substantial survival prolongation in rats bearing multi-focal lesions of HCC in the liver was achieved after hepatic artery infusion of this recombinant vector, rVSV-A239L (FIG. 22). These results confirm that the anti-tumor efficacy of oncolytic viruses can be substantively enhanced by vector-mediated expression of NF-κB suppressive genes.

In addition to African swine fever virus, there are several other viruses that are known to encode NF-κB inhibitory genes. For example, the poxviruses encode at least two proteins that interfere with activation of NF-κB. The A52R protein potently blocks IL-1- and TLR4-mediated activation of NF-κB, while N1L targets the IKK complex. Bowie et al., Proc. Natl. Acad. Sci. U.S.A. 97:10162-10167 (2000) and DiPerna et al., J. Biol. Chem. 279:36570-36578 (2004).

Another example is the human immunodeficiency virus (HIV) accessory protein, Vpu, which interferes with degradation of IκB and suppresses NF-κB-dependent expression of antiapoptotic factors. Akari et al., J. Exp. Med. 194:1299-1311 (2001) and Bour et al., J. Biol. Chem. 276:15920-15928 (2001).

Thirdly, the Torque teno virus ORF2 protein suppresses NF-κB pathways by interacting with IKKs, and blocking nuclear transport of NF-κB by inhibiting IκB protein degradation.

Additionally, a cellular gene that suppresses NF-κB has also been generated. The IκB super repressor is a mutant form of IκB, in which serine to alanine mutations have been introduced at amino acids 32 and 36. Wang et al., Science 274:784-787 (1996). This modified form of IκB is resistant to signal-induced phosphorylation and subsequent proteosome-mediated degradation, and thereby prevents activation of NF-κB. Uesugi et al., Hepatology 34:1149-1157 (2001) and Hellerbrand et al., Hepatology 27:1285-1295 (1998).

All of these NF-κB suppressive genes of viral and cellular origins can be inserted into oncolytic viruses in the manner described herein to achieve enhanced anti-tumor efficacy.

1. A recombinant oncolytic virus, comprising an oncolytic virus or a recombinant variant of an oncolytic virus and a heterologous nucleic acid sequence encoding an inhibitor of inflammatory or innate immune cell migration or function, wherein said heterologous nucleic acid sequence is incorporated within the genetic material of said oncolytic virus or recombinant variant of an oncolytic virus.

2. The recombinant oncolytic virus of claim 1 wherein said oncolytic virus is selected from the group consisting of vesicular stomatitis virus (VSV), Newcastle disease virus (NDV), retrovirus, reovirus, measles virus, Sinbis virus, influenza virus, herpes simplex virus, vaccinia virus, and adenovirus.

3. The recombinant oncolytic virus of claim 1 and wherein said recombinant variant of an oncolytic virus is a recombinant variant of a virus selected from the group consisting of vesicular stomatitis virus (VSV), Newcastle disease virus (NDV), retrovirus, reovirus, measles virus, Sinbis virus, influenza virus, herpes simplex virus, vaccinia virus, and adenovirus.

4. The recombinant oncolytic virus according to claim 1 wherein said inhibitor of inflammatory cell migration or function is selected from the group consisting of a natural killer cell inhibitor, a chemokine binding protein, and an NF-κB inhibitor.

5. The recombinant oncolytic virus according to claim 4 wherein said natural killer cell inhibitor, said chemokine binding protein, or said NF-κB inhibitor is a viral protein, a bacterial protein, a fungal protein, a parasitic protein, or a eukaryotic protein.

6. The recombinant oncolytic virus according to claim 5 wherein said inhibitor of inflammatory cell migration or function is a chemokine binding protein or a truncated variant thereof.

7.-15. (canceled)

16. The recombinant oncolytic virus according to claim 5 wherein said inhibitor of inflammatory cell migration or function is a natural killer cell inhibitor or a truncated variant thereof.

17.-19. (canceled)

20. The recombinant oncolytic virus according to claim 5 wherein said inhibitor of inflammatory cell migration or function is an NF-κB inhibitor or a truncated variant thereof.

21.-23. (canceled)

24. The recombinant oncolytic virus according to claim 1, further comprising a heterologous viral internal ribosome entry site (IRES) that is neuronally-silent and operably linked to at least one nucleic acid sequence that encodes an oncolytic virus polypeptide.

25. The recombinant oncolytic virus according to claim 24 wherein the oncolytic virus polypeptide is one or more of an oncolytic virus polymerase, an oncolytic virus structural protein, or an oncolytic virus glycoprotein.

26. The recombinant oncolytic virus according to claim 24 wherein the recombinant oncolytic virus comprises two or more IRESs and each is operably linked to a different nucleic acid sequence that encodes an oncolytic virus polypeptide.

27.-28. (canceled)

29. The recombinant oncolytic virus according to claim 24 wherein the IRES is a picornavirus IRES.

30.-33. (canceled)

34. A recombinant oncolytic virus, comprising an oncolytic virus or a recombinant variant of an oncolytic virus and a heterologous nucleic acid sequence encoding an inhibitor of inflammatory or innate immune cell migration or function, wherein said heterologous nucleic acid sequence is incorporated within the genetic material of said oncolytic virus or recombinant variant of an oncolytic virus, said recombinant oncolytic virus further comprising a heterologous nucleic acid sequence encoding a viral internal ribosome entry site (IRES) that is neuronally-silent and operably linked to a nucleic acid sequence that encodes an oncolytic virus polypeptide.

35.-52. (canceled)

53. The recombinant oncolytic virus according to claim 34 wherein the recombinant oncolytic virus comprises two or more IRESs and each is operably linked to a different nucleic acid sequence that encodes an oncolytic virus polypeptide.

54.-55. (canceled)

56. The recombinant oncolytic virus according to claim 34 wherein the IRES is a picornavirus IRES.

57.-60. (canceled)

61. A method of inhibiting the growth or promoting the killing of a tumor cell, said method comprising the step of contacting said tumor cell with a recombinant oncolytic virus according to claim 1 at a multiplicity of infection sufficient to inhibit the growth or kill the tumor cell.

62. The method according to claim 61 wherein said tumor cell is selected from the group consisting of a hepatocellular carcinoma (HCC) cell, a colorectal cancer cell, a breast cancer cell, a lung cancer cell, a head and neck cancer cell, a brain cancer cell, a leukemia cell, a prostate cancer cell, a bladder cancer cell, and an ovarian cancer cell.

63.-82. (canceled)

83. A method of inhibiting the growth or promoting the killing of a tumor cell, said method comprising the step of contacting said tumor cell with a recombinant oncolytic virus according to claim 34 at a multiplicity of infection sufficient to inhibit the growth or kill the tumor cell.

84. The method according to claim 83 wherein said tumor cell is selected from the group consisting of a hepatocellular carcinoma (HCC) cell, a colorectal cancer cell, a breast cancer cell, a lung cancer cell, a head and neck cancer cell, a brain cancer cell, a leukemia cell, a prostate cancer cell, a bladder cancer cell, and an ovarian cancer cell.
